SEQUENCE LISTING (1952 sequences)

SEQ ID NO: 1 - protein, 735 aa, synthetic construct [sequence omitted]

SEQ ID NO: 2 - protein, 11 aa, synthetic construct
GNCLCTFLTRQ

SEQ ID NO: 3 - protein, 11 aa, synthetic construct
GNFQLESMNRQ

SEQ ID NO: 4 - protein, 11 aa, synthetic construct
GNPVAGLPPRQ

SEQ ID NO: 5 - protein, 11 aa, synthetic construct
GNVVSGLAARQ

SEQ ID NO: 6 - protein, 11 aa, synthetic construct
GNLAGKPPPRQ

SEQ ID NO: 7 - protein, 11 aa, synthetic construct
GNIMGPHMTRQ

SEQ ID NO: 8 - protein, 11 aa, synthetic construct
GNLLGTALTRQ

SEQ ID NO: 9 - protein, 11 aa, synthetic construct
GNYQLERMNRQ

SEQ ID NO: 10 - protein, 11 aa, synthetic construct
GNLLGTSMQRQ

SEQ ID NO: 11 - protein, 11 aa, synthetic construct
GNLLGKSMERQ

SEQ ID NO: 12 - protein, 11 aa, synthetic construct
GNILGSSLERL

SEQ ID NO: 13 - protein, 11 aa, synthetic construct
GNILSPSLARQ

SEQ ID NO: 14 - protein, 11 aa, synthetic construct
GNLLGANMVRQ

SEQ ID NO: 15 - protein, 11 aa, synthetic construct
GNIVAGLKSRQ

SEQ ID NO: 16 - protein, 11 aa, synthetic construct
GNLLGPYMQRQ

SEQ ID NO: 17 - protein, 11 aa, synthetic construct
GNVLGYNLQRQ

SEQ ID NO: 18 - protein, 11 aa, synthetic construct
GNLLGKTMERQ

SEQ ID NO: 19 - protein, 11 aa, synthetic construct
GNLLGNALTRQ

SEQ ID NO: 20 - protein, 11 aa, synthetic construct
GNILGITLARQ

SEQ ID NO: 21 - protein, 11 aa, synthetic construct
GNILGSTLARQ

SEQ ID NO: 22 - protein, 11 aa, synthetic construct
GNQVPGLGVRQ

SEQ ID NO: 23 - protein, 11 aa, synthetic construct
GNHNVSIPARQ

SEQ ID NO: 24 - protein, 11 aa, synthetic construct
GNPSRECGLRQ

SEQ ID NO: 25 - protein, 11 aa, synthetic construct
GNLLGPNLSRQ

SEQ ID NO: 26 - protein, 11 aa, synthetic construct
GNLLGTAMTRQ

SEQ ID NO: 27 - protein, 11 aa, synthetic construct
GNVVSGIAARQ

SEQ ID NO: 28 - protein, 11 aa, synthetic construct
GNIMGPHLARQ

SEQ ID NO: 29 - protein, 11 aa, synthetic construct
GSLLGPSMQRQ

SEQ ID NO: 30 - protein, 11 aa, synthetic construct
GNVVSGLAARL

SEQ ID NO: 31 - protein, 11 aa, synthetic construct
GNIMGPHLTRQ

SEQ ID NO: 32 - protein, 11 aa, synthetic construct
GNLFGPSMARQ

SEQ ID NO: 33 - protein, 11 aa, synthetic construct
GNVVSGLSARQ

SEQ ID NO: 34 - protein, 11 aa, synthetic construct
GNQVPGLGVRL

SEQ ID NO: 35 - protein, 11 aa, synthetic construct
GNILGSGLTRQ

SEQ ID NO: 36 - protein, 11 aa, synthetic construct
GNIMGPLLTRQ

SEQ ID NO: 37 - protein, 11 aa, synthetic construct
GNLLGTAMERQ

SEQ ID NO: 38 - protein, 11 aa, synthetic construct
GNGLGPALVRQ

SEQ ID NO: 39 - protein, 11 aa, synthetic construct
GNILGSSLERQ

SEQ ID NO: 40 - protein, 11 aa, synthetic construct
GNIVAGLAQRQ

SEQ ID NO: 41 - protein, 11 aa, synthetic construct
GNPVAGLTPRQ

SEQ ID NO: 42 - protein, 11 aa, synthetic construct
GNIFGRDMARQ

SEQ ID NO: 43 - protein, 11 aa, synthetic construct
GNILGPSNARQ

SEQ ID NO: 44 - protein, 11 aa, synthetic construct
GNLLGPTMQRQ

SEQ ID NO: 45 - protein, 11 aa, synthetic construct
GNMSQKIPKRQ

SEQ ID NO: 46 - protein, 11 aa, synthetic construct
GNIVSGLAGRQ

SEQ ID NO: 47 - protein, 11 aa, synthetic construct
GNTVPGLGHRQ

SEQ ID NO: 48 - protein, 11 aa, synthetic construct
GNLLGTSLTRQ

SEQ ID NO: 49 - protein, 11 aa, synthetic construct
GNLLGHSMQRQ

SEQ ID NO: 50 - protein, 11 aa, synthetic construct
GNLLGSNLERQ

SEQ ID NO: 51 - protein, 11 aa, synthetic construct
GNPVPGLSSRQ

SEQ ID NO: 52 - protein, 11 aa, synthetic construct
GNFQLERMNRQ

SEQ ID NO: 53 - protein, 11 aa, synthetic construct
GNVLGSALSRQ

SEQ ID NO: 54 - protein, 11 aa, synthetic construct
GNVLGHILARQ

SEQ ID NO: 55 - protein, 11 aa, synthetic construct
GNVVPGLSYRQ

SEQ ID NO: 56 - protein, 11 aa, synthetic construct
GNILGITLARQ

SEQ ID NO: 57 - protein, 11 aa, synthetic construct
GNILGSTLARQ

SEQ ID NO: 58 - protein, 11 aa, synthetic construct
GNQVPGLGVRQ

SEQ ID NO: 59 - protein, 11 aa, synthetic construct
GNHNVSIPARQ

SEQ ID NO: 60 - protein, 11 aa, synthetic construct
GNPSRECGLRQ

SEQ ID NO: 61 - protein, 11 aa, synthetic construct
GNLLGPNLSRQ

SEQ ID NO: 62 - protein, 11 aa, synthetic construct
GNLLGTAMTRQ

SEQ ID NO: 63 - protein, 11 aa, synthetic construct
GNVVSGIAARQ

SEQ ID NO: 64 - protein, 11 aa, synthetic construct
GNIMGPHLARQ

SEQ ID NO: 65 - protein, 11 aa, synthetic construct
GSLLGPSMQRQ

SEQ ID NO: 66 - protein, 11 aa, synthetic construct
GNVVSGLAARL

SEQ ID NO: 67 - protein, 11 aa, synthetic construct
GNIMGPHLTRQ

SEQ ID NO: 68 - protein, 11 aa, synthetic construct
GNLFGPSMARQ

SEQ ID NO: 69 - protein, 11 aa, synthetic construct
GNVVSGLSARQ

SEQ ID NO: 70 - protein, 11 aa, synthetic construct
GNQVPGLGVRL

SEQ ID NO: 71 - protein, 11 aa, synthetic construct
GNILGSGLTRQ

SEQ ID NO: 72 - protein, 11 aa, synthetic construct
GNIMGPLLTRQ

SEQ ID NO: 73 - protein, 11 aa, synthetic construct
GNLLGTAMERQ

SEQ ID NO: 74 - protein, 11 aa, synthetic construct
GNGLGPALVRQ

SEQ ID NO: 75 - protein, 11 aa, synthetic construct
GNILGSSLERQ

SEQ ID NO: 76 - protein, 11 aa, synthetic construct
GNIVAGLAQRQ

SEQ ID NO: 77 - protein, 11 aa, synthetic construct
GNPVAGLTPRQ

SEQ ID NO: 78 - protein, 11 aa, synthetic construct
GNIFGRDMARQ

SEQ ID NO: 79 - protein, 11 aa, synthetic construct
GNILGPSNARQ

SEQ ID NO: 80 - protein, 11 aa, synthetic construct
GNLLGPTMQRQ

SEQ ID NO: 81 - protein, 11 aa, synthetic construct
GNMSQKIPKRQ

SEQ ID NO: 82 - protein, 11 aa, synthetic construct
GNIVSGLAGRQ

SEQ ID NO: 83 - protein, 11 aa, synthetic construct
GNTVPGLGHRQ

SEQ ID NO: 84 - protein, 11 aa, synthetic construct
GNLLGTSLTRQ

SEQ ID NO: 85 - protein, 11 aa, synthetic construct
GNLLGHSMQRQ

SEQ ID NO: 86 - protein, 11 aa, synthetic construct
GNLLGSNLERQ

SEQ ID NO: 87 - protein, 11 aa, synthetic construct
GNPVPGLSSRQ

SEQ ID NO: 88 - protein, 11 aa, synthetic construct
GNFQLERMNRQ

SEQ ID NO: 89 - protein, 11 aa, synthetic construct
GNVLGSALSRQ

SEQ ID NO: 90 - protein, 11 aa, synthetic construct
GNVLGHILARQ

SEQ ID NO: 91 - protein, 11 aa, synthetic construct
GNVVPGLSYRQ

SEQ ID NO: 92 - protein, 11 aa, synthetic construct
GNVMGPALTRQ

SEQ ID NO: 93 - protein, 11 aa, synthetic construct
GNLLGPSMQRQ

SEQ ID NO: 94 - protein, 11 aa, synthetic construct
GNVLGHSLSRQ

SEQ ID NO: 95 - protein, 11 aa, synthetic construct
GNSVAGLGPRQ

SEQ ID NO: 96 - protein, 11 aa, synthetic construct
GNHVPGLGVRQ

SEQ ID NO: 97 - protein, 11 aa, synthetic construct
GNVLGHMLARQ

SEQ ID NO: 98 - protein, 11 aa, synthetic construct
GNIVSGLSGRQ

SEQ ID NO: 99 - protein, 11 aa, synthetic construct
GNIVAGLSSRQ

SEQ ID NO: 100 - protein, 11 aa, synthetic construct
GNLVPGLSARQ

SEQ ID NO: 101 - protein, 11 aa, synthetic construct
GNFQLERLNRQ

SEQ ID NO: 102 - protein, 11 aa, synthetic construct
GNVLGHMLARL

SEQ ID NO: 103 - protein, 11 aa, synthetic construct
GNWVPPQTPRQ

SEQ ID NO: 104 - protein, 11 aa, synthetic construct
GNVLGHLLARQ

SEQ ID NO: 105 - protein, 11 aa, synthetic construct
GNVLGNMLARQ

SEQ ID NO: 106 - protein, 11 aa, synthetic construct
GSILGSSLTRQ

SEQ ID NO: 107 - protein, 11 aa, synthetic construct
GNVVSGLASRQ

SEQ ID NO: 108 - protein, 11 aa, synthetic construct
GNVVPGLSYRL

SEQ ID NO: 109 - protein, 11 aa, synthetic construct
GNVLGHMLERQ

SEQ ID NO: 110 - protein, 11 aa, synthetic construct
GNVDSNQVSRQ

SEQ ID NO: 111 - protein, 11 aa, synthetic construct
GNIVSGLSARQ

SEQ ID NO: 112 - protein, 11 aa, synthetic construct
GNLLGTALSRQ

SEQ ID NO: 113 - protein, 11 aa, synthetic construct
GNILGTTLSRQ

SEQ ID NO: 114 - protein, 11 aa, synthetic construct
GNVFGSSLVRQ

SEQ ID NO: 115 - protein, 11 aa, synthetic construct
GNLLGSALNRQ

SEQ ID NO: 116 - protein, 11 aa, synthetic construct
GNLLGPTLSRQ

SEQ ID NO: 117 - protein, 11 aa, synthetic construct
GNILGHGMLRQ

SEQ ID NO: 118 - protein, 11 aa, synthetic construct
GNLLGPSMQRL

SEQ ID NO: 119 - protein, 11 aa, synthetic construct
GNNVAASPSRQ

SEQ ID NO: 120 - protein, 11 aa, synthetic construct
GNNFTADPLRQ

SEQ ID NO: 121 - protein, 11 aa, synthetic construct
GNGDSGISSRQ

SEQ ID NO: 122 - protein, 11 aa, synthetic construct
GNILGSSLTRQ

SEQ ID NO: 123 - protein, 11 aa, synthetic construct
GNTNTTHLERQ

SEQ ID NO: 124 - protein, 11 aa, synthetic construct
GNIMGAHLTRQ

SEQ ID NO: 125 - protein, 11 aa, synthetic construct
GNVLGVHLTRQ

SEQ ID NO: 126 - protein, 11 aa, synthetic construct
GNILGSTLTRQ

SEQ ID NO: 127 - protein, 11 aa, synthetic construct
GNILGELLSRQ

SEQ ID NO: 128 - protein, 11 aa, synthetic construct
GNILGNIMSRQ

SEQ ID NO: 129 - protein, 11 aa, synthetic construct
GNVLGYNMQRQ

SEQ ID NO: 130 - protein, 11 aa, synthetic construct
GNILGNILSRQ

SEQ ID NO: 131 - protein, 11 aa, synthetic construct
GNADPPQWKRQ

SEQ ID NO: 132 - protein, 11 aa, synthetic construct
GNLLGTQLARQ

SEQ ID NO: 133 - protein, 11 aa, synthetic construct
GNVLGSAMKRQ

SEQ ID NO: 134 - protein, 11 aa, synthetic construct
GNLLGSSLSRQ

SEQ ID NO: 135 - protein, 11 aa, synthetic construct
GNFCSVNKERQ

SEQ ID NO: 136 - protein, 11 aa, synthetic construct
GNLLGPNLCRQ

SEQ ID NO: 137 - protein, 11 aa, synthetic construct
GNFQLERMNRL

SEQ ID NO: 138 - protein, 11 aa, synthetic construct
GNPVSGLPPRQ

SEQ ID NO: 139 - protein, 11 aa, synthetic construct
GNLLGPNLSRL

SEQ ID NO: 140 - protein, 11 aa, synthetic construct
GNLLGRNLSRQ

SEQ ID NO: 141 - protein, 11 aa, synthetic construct
GNSIPPAAKRQ

SEQ ID NO: 142 - protein, 11 aa, synthetic construct
GNVLGFNMQRQ

SEQ ID NO: 143 - protein, 11 aa, synthetic construct
GNPIPGLGARQ

SEQ ID NO: 144 - protein, 11 aa, synthetic construct
GNLLGTNLSRQ

SEQ ID NO: 145 - protein, 11 aa, synthetic construct
GSLLGPNLSRQ

SEQ ID NO: 146 - protein, 11 aa, synthetic construct
GNAPPLQFSRQ

SEQ ID NO: 147 - protein, 11 aa, synthetic construct
GNVMGSALARQ

SEQ ID NO: 148 - protein, 11 aa, synthetic construct
GNLLGHNLSRQ

SEQ ID NO: 149 - protein, 11 aa, synthetic construct
GNLLGSSLTRQ

SEQ ID NO: 150 - protein, 11 aa, synthetic construct
GNVFGPGLIRQ

SEQ ID NO: 151 - protein, 11 aa, synthetic construct
GNIMGPTLTRQ

SEQ ID NO: 152 - protein, 11 aa, synthetic construct
GNVHGPALIRQ

SEQ ID NO: 153 - protein, 11 aa, synthetic construct
GNILGSSLTRH

SEQ ID NO: 154 - protein, 11 aa, synthetic construct
GNMLGPSMQRQ

SEQ ID NO: 155 - protein, 11 aa, synthetic construct
GNIMGPHLTRL

SEQ ID NO: 156 - protein, 11 aa, synthetic construct
GNQNPRIEQRQ

SEQ ID NO: 157 - protein, 11 aa, synthetic construct
GNLLGPHLSRL

SEQ ID NO: 158 - protein, 11 aa, synthetic construct
GNLLGHALTRQ

SEQ ID NO: 159 - protein, 11 aa, synthetic construct
GNIMGVSLNRQ

SEQ ID NO: 160 - protein, 11 aa, synthetic construct
GNIMGTHLTRQ

SEQ ID NO: 161 - protein, 11 aa, synthetic construct
GNLVPGIPARQ

SEQ ID NO: 162 - protein, 11 aa, synthetic construct
GNQILAPLSRQ

SEQ ID NO: 163 - protein, 11 aa, synthetic construct
GNNVTTFPPRQ

SEQ ID NO: 164 - protein, 11 aa, synthetic construct
GNLLGPMLSRQ

SEQ ID NO: 165 - protein, 11 aa, synthetic construct
GNLLGQTLSRQ

SEQ ID NO: 166 - protein, 11 aa, synthetic construct
GNHASQFTFRQ

SEQ ID NO: 167 - protein, 11 aa, synthetic construct
GNTVPGLGHRL

SEQ ID NO: 168 - protein, 11 aa, synthetic construct
GNSLPPPTKRQ

SEQ ID NO: 169 - protein, 11 aa, synthetic construct
GNNVSQQPVRQ

SEQ ID NO: 170 - protein, 11 aa, synthetic construct
GNNIAHMQLRQ

SEQ ID NO: 171 - protein, 11 aa, synthetic construct
GNILGEVLTRQ

SEQ ID NO: 172 - protein, 11 aa, synthetic construct
GNILGPHLDRQ

SEQ ID NO: 173 - protein, 11 aa, synthetic construct
GIIMGPHLTRQ

SEQ ID NO: 174 - protein, 11 aa, synthetic construct
GNVLGSALNRQ

SEQ ID NO: 175 - protein, 11 aa, synthetic construct
GNILGSSLTRL

SEQ ID NO: 176 - protein, 11 aa, synthetic construct
GNLLGHMLARQ

SEQ ID NO: 177 - protein, 11 aa, synthetic construct
GNILGTSLTRQ

SEQ ID NO: 178 - protein, 11 aa, synthetic construct
GNILGSSLVRQ

SEQ ID NO: 179 - protein, 11 aa, synthetic construct
GNLLGPNLTRQ

SEQ ID NO: 180 - protein, 11 aa, synthetic construct
GNSDNATHSRQ

SEQ ID NO: 181 - protein, 11 aa, synthetic construct
GNVSPPTFTRQ

SEQ ID NO: 182 - protein, 11 aa, synthetic construct
GNNITGTPVRQ

SEQ ID NO: 183 - protein, 11 aa, synthetic construct
GNLLGPTLSRL

SEQ ID NO: 184 - protein, 11 aa, synthetic construct
GNSVPGLGHRQ

SEQ ID NO: 185 - protein, 11 aa, synthetic construct
GNIVAGLAQRL

SEQ ID NO: 186 - protein, 11 aa, synthetic construct
GNMLGPNLSRQ

SEQ ID NO: 187 - protein, 11 aa, synthetic construct
GIILGSSLTRQ

SEQ ID NO: 188 - protein, 11 aa, synthetic construct
GNVVSGLADRQ

SEQ ID NO: 189 - protein, 11 aa, synthetic construct
GSDNRLILDRQ

SEQ ID NO: 190 - protein, 11 aa, synthetic construct
GNILGPHLTRQ

SEQ ID NO: 191 - protein, 11 aa, synthetic construct
GNNLSHDPLRQ

SEQ ID NO: 192 - protein, 11 aa, synthetic construct
GNLLGPGLTRQ

SEQ ID NO: 193 - protein, 11 aa, synthetic construct
GNEDSNRTMRQ

SEQ ID NO: 194 - protein, 11 aa, synthetic construct
GNNLSASPLRQ

SEQ ID NO: 195 - protein, 11 aa, synthetic construct
GNGECHLSFRQ

SEQ ID NO: 196 - protein, 11 aa, synthetic construct
GNQNPKIPSRQ

SEQ ID NO: 197 - protein, 11 aa, synthetic construct
GNVVPGLSNRQ

SEQ ID NO: 198 - protein, 11 aa, synthetic construct
GNIIPGLVARQ

SEQ ID NO: 199 - protein, 11 aa, synthetic construct
GNMQLSSLNRQ

SEQ ID NO: 200 - protein, 11 aa, synthetic construct
GNLLGTALTRL

SEQ ID NO: 201 - protein, 11 aa, synthetic construct
GNVLGHMLSRQ

SEQ ID NO: 202 - protein, 11 aa, synthetic construct
GNILGEVLSRQ

SEQ ID NO: 203 - protein, 11 aa, synthetic construct
GNLPAGIHGRQ

SEQ ID NO: 204 - protein, 11 aa, synthetic construct
GNIMGHHLTRQ

SEQ ID NO: 205 - protein, 11 aa, synthetic construct
GNLLGPSMKRQ

SEQ ID NO: 206 - protein, 11 aa, synthetic construct
GNLGTPFTPRQ

SEQ ID NO: 207 - protein, 11 aa, synthetic construct
GNPVPGLTTRQ

SEQ ID NO: 208 - protein, 11 aa, synthetic construct
GNIVSGLAQRQ

SEQ ID NO: 209 - protein, 11 aa, synthetic construct
GNVLGHKLARQ

SEQ ID NO: 210 - protein, 11 aa, synthetic construct
GNNVTRAELRQ

SEQ ID NO: 211 - protein, 11 aa, synthetic construct
GNFLGPSMQRQ

SEQ ID NO: 212 - protein, 11 aa, synthetic construct
GNNISQDPPRQ

SEQ ID NO: 213 - protein, 11 aa, synthetic construct
GNTVPGLGLRQ

SEQ ID NO: 214 - protein, 11 aa, synthetic construct
GNFLGPNLSRQ

SEQ ID NO: 215 - protein, 11 aa, synthetic construct
GNIIPGLVARL

SEQ ID NO: 216 - protein, 11 aa, synthetic construct
GNNTVRAELRQ

SEQ ID NO: 217 - protein, 11 aa, synthetic construct
GNMASTLGPRQ

SEQ ID NO: 218 - protein, 11 aa, synthetic construct
GDLLGPSMQRQ

SEQ ID NO: 219 - protein, 11 aa, synthetic construct
GNSRSFGNPRQ

SEQ ID NO: 220 - protein, 11 aa, synthetic construct
GNVDRNSTSRQ

SEQ ID NO: 221 - protein, 11 aa, synthetic construct
GNIVAGLSQRQ

SEQ ID NO: 222 - protein, 11 aa, synthetic construct
GNILGTSLVRQ

SEQ ID NO: 223 - protein, 11 aa, synthetic construct
GNLLGPEMTRQ

SEQ ID NO: 224 - protein, 11 aa, synthetic construct
GNLLGPVMARQ

SEQ ID NO: 225 - protein, 11 aa, synthetic construct
GNVIPGLVVRQ

SEQ ID NO: 226 - protein, 11 aa, synthetic construct
GNIVAGLTSRQ

SEQ ID NO: 227 - protein, 11 aa, synthetic construct
GNLTGNQMRRQ

SEQ ID NO: 228 - protein, 11 aa, synthetic construct
GNNMSASPLRQ

SEQ ID NO: 229 - protein, 11 aa, synthetic construct
GNLSTTQLSRQ

SEQ ID NO: 230 - protein, 11 aa, synthetic construct
GNSVKDWTTRQ

SEQ ID NO: 231 - protein, 11 aa, synthetic construct
GNILGEVMSRQ

SEQ ID NO: 232 - protein, 11 aa, synthetic construct
GNLFGNSMVRQ

SEQ ID NO: 233 - protein, 11 aa, synthetic construct
GNPVSGLASRQ

SEQ ID NO: 234 - protein, 11 aa, synthetic construct
GNILGTALTRQ

SEQ ID NO: 235 - protein, 11 aa, synthetic construct
GNLLGPNLIRQ

SEQ ID NO: 236 - protein, 11 aa, synthetic construct
GNLLGKSLTRQ

SEQ ID NO: 237 - protein, 11 aa, synthetic construct
GNSSNSVSLRQ

SEQ ID NO: 238 - protein, 11 aa, synthetic construct
GNILGSSLMRQ

SEQ ID NO: 239 - protein, 11 aa, synthetic construct
GNVALSMHARQ

SEQ ID NO: 240 - protein, 11 aa, synthetic construct
GNIMGPNLTRQ

SEQ ID NO: 241 - protein, 11 aa, synthetic construct
GNNIVAQQLRQ

SEQ ID NO: 242 - protein, 11 aa, synthetic construct
GNPRVSGSQRQ

SEQ ID NO: 243 - protein, 11 aa, synthetic construct
GNSLDQSLPRQ

SEQ ID NO: 244 - protein, 11 aa, synthetic construct
GNADSGTPERQ

SEQ ID NO: 245 - protein, 11 aa, synthetic construct
GNILGATLTRQ

SEQ ID NO: 246 - protein, 11 aa, synthetic construct
GNKNVSIAERQ

SEQ ID NO: 247 - protein, 11 aa, synthetic construct
GNNEPHMGARQ

SEQ ID NO: 248 - protein, 11 aa, synthetic construct
GNSAITSQDRQ

SEQ ID NO: 249 - protein, 11 aa, synthetic construct
GNSPNEAQFRQ

SEQ ID NO: 250 - protein, 11 aa, synthetic construct
GNNPFLASQRQ

SEQ ID NO: 251 - protein, 11 aa, synthetic construct
GNNMSATPLRQ

SEQ ID NO: 252 - protein, 11 aa, synthetic construct
GNFYATSTSRQ

SEQ ID NO: 253 - protein, 11 aa, synthetic construct
GNILGTHLTRQ

SEQ ID NO: 254 - protein, 11 aa, synthetic construct
GNQQAHEVGRQ

SEQ ID NO: 255 - protein, 11 aa, synthetic construct
GNPALYIFIRQ

SEQ ID NO: 256 - protein, 11 aa, synthetic construct
GNVLGLMLARQ

SEQ ID NO: 257 - protein, 11 aa, synthetic construct
GNLLGPYLSRQ

SEQ ID NO: 258 - protein, 11 aa, synthetic construct
GNVLGYYMQRQ

SEQ ID NO: 259 - protein, 11 aa, synthetic construct
GNLPPICHVRQ

SEQ ID NO: 260 - protein, 11 aa, synthetic construct
GNISGNEILRQ

SEQ ID NO: 261 - protein, 11 aa, synthetic construct
GKVLGHMLARQ

SEQ ID NO: 262 - protein, 11 aa, synthetic construct
GNGGICPGARQ

SEQ ID NO: 263 - protein, 11 aa, synthetic construct
GNVSIYTSSRQ

SEQ ID NO: 264 - protein, 11 aa, synthetic construct
GNLLGPTLTRQ

SEQ ID NO: 265 - protein, 11 aa, synthetic construct
GNPWMTSYVRQ

SEQ ID NO: 266 - protein, 11 aa, synthetic construct
GNGHRNNHLRQ

SEQ ID NO: 267 - protein, 11 aa, synthetic construct
GNTHCSYPDRQ

SEQ ID NO: 268 - protein, 11 aa, synthetic construct
GNSLYMSSLRQ

SEQ ID NO: 269 - protein, 11 aa, synthetic construct
GNALSCLWYRQ

SEQ ID NO: 270 - protein, 11 aa, synthetic construct
GNILGNFMSRQ

SEQ ID NO: 271 - protein, 11 aa, synthetic construct
GNLVTRGSMRQ

SEQ ID NO: 272 - protein, 11 aa, synthetic construct
GNVLPGLNARQ

SEQ ID NO: 273 - protein, 11 aa, synthetic construct
GNMLTCSVSRQ

SEQ ID NO: 274 - protein, 11 aa, synthetic construct
GNLSIRTHSRQ

SEQ ID NO: 275 - protein, 11 aa, synthetic construct
GNTLQNIYSRQ

SEQ ID NO: 276 - protein, 11 aa, synthetic construct
GNITTPHKQRQ

SEQ ID NO: 277 - protein, 11 aa, synthetic construct
GNVRPITFLRQ

SEQ ID NO: 278 - protein, 11 aa, synthetic construct
GNSDSFSLSRQ

SEQ ID NO: 279 - protein, 11 aa, synthetic construct
GNMPNTFLSRQ

SEQ ID NO: 280 - protein, 11 aa, synthetic construct
GNILGEVLSRL

SEQ ID NO: 281 - protein, 11 aa, synthetic construct
GNQSRVTRVRQ

SEQ ID NO: 282 - protein, 11 aa, synthetic construct
GNNVTSQRTRQ

SEQ ID NO: 283 - protein, 11 aa, synthetic construct
GNILGYSLTRQ

SEQ ID NO: 284 - protein, 11 aa, synthetic construct
GNFGTNRTNRQ

SEQ ID NO: 285 - protein, 11 aa, synthetic construct
GNLNLLIGLRQ

SEQ ID NO: 286 - protein, 11 aa, synthetic construct
GNLVSKTKSRQ

SEQ ID NO: 287 - protein, 11 aa, synthetic construct
GNGGFTIYERQ

SEQ ID NO: 288 - protein, 11 aa, synthetic construct
GNDPPLPYPRQ

SEQ ID NO: 289 - protein, 11 aa, synthetic construct
GNGMMVTVMRQ

SEQ ID NO: 290 - protein, 11 aa, synthetic construct
GNGSRYTPTRQ

SEQ ID NO: 291 - protein, 11 aa, synthetic construct
GNQSLLCSERQ

SEQ ID NO: 292 - protein, 11 aa, synthetic construct
GNDSSYSLIRQ

SEQ ID NO: 293 - protein, 11 aa, synthetic construct
GNPHINKTLRQ

SEQ ID NO: 294 - protein, 11 aa, synthetic construct
GNLLGPSMERQ

SEQ ID NO: 295 - protein, 11 aa, synthetic construct
GNPAPHCSLRQ

SEQ ID NO: 296 - protein, 11 aa, synthetic construct
GNLLGPSMQRR

SEQ ID NO: 297 - protein, 11 aa, synthetic construct
GNLLGPLLSRQ

SEQ ID NO: 298 - protein, 11 aa, synthetic construct
GNQYQSLGPRQ

SEQ ID NO: 299 - protein, 11 aa, synthetic construct
GNLLGLALTRQ

SEQ ID NO: 300 - protein, 11 aa, synthetic construct
GNNTHTYMHRQ

SEQ ID NO: 301 - protein, 11 aa, synthetic construct
GNRLSTSPLRQ

SEQ ID NO: 302 - protein, 11 aa, synthetic construct
GNTPSSQDSRQ

SEQ ID NO: 303 - protein, 11 aa, synthetic construct
GNSISIKRSRQ

SEQ ID NO: 304 - protein, 11 aa, synthetic construct
GNPSNTESPRQ

SEQ ID NO: 305 - protein, 11 aa, synthetic construct
GNYQSTMYARQ

SEQ ID NO: 306 - protein, 11 aa, synthetic construct
GNLNESYYSRQ

SEQ ID NO: 307 - protein, 11 aa, synthetic construct
GNMRLALPGRQ

SEQ ID NO: 308 - protein, 11 aa, synthetic construct
GNPSSRQQLRL

SEQ ID NO: 309 - protein, 11 aa, synthetic construct
GNSAIFSVPRQ

SEQ ID NO: 310 - protein, 11 aa, synthetic construct
GNLLGPSMLRQ

SEQ ID NO: 311 - protein, 11 aa, synthetic construct
GNSSSHEYSRQ

SEQ ID NO: 312 - protein, 11 aa, synthetic construct
GNLSTYQLSRQ

SEQ ID NO: 313 - protein, 11 aa, synthetic construct
GNAMNLLAYRQ

SEQ ID NO: 314 - protein, 11 aa, synthetic construct
GNQGTQQYERQ

SEQ ID NO: 315 - protein, 11 aa, synthetic construct
GNGRSSSDERQ

SEQ ID NO: 316 - protein, 11 aa, synthetic construct
GNTDQTATDRQ

SEQ ID NO: 317 - protein, 11 aa, synthetic construct
GNSLGPTLVRQ

SEQ ID NO: 318 - protein, 11 aa, synthetic construct
GNTIPGLQVRQ

SEQ ID NO: 319 - protein, 11 aa, synthetic construct
SNTRYLVPQRQ

SEQ ID NO: 320 - protein, 11 aa, synthetic construct
GNYYSKLPLRQ

SEQ ID NO: 321 - protein, 11 aa, synthetic construct
GNYSVHLYPRQ

SEQ ID NO: 322 - protein, 11 aa, synthetic construct
GNYLAFTRFRQ

SEQ ID NO: 323 - protein, 11 aa, synthetic construct
GNWYLGEPVRQ

SEQ ID NO: 324 - protein, 11 aa, synthetic construct
GNWTISGINRQ

SEQ ID NO: 325 - protein, 11 aa, synthetic construct
GNWHFHQSERQ

SEQ ID NO: 326 - protein, 11 aa, synthetic construct
GNVVSCSYDRQ

SEQ ID NO: 327 - protein, 11 aa, synthetic construct
GNVSVECTRRQ

SEQ ID NO: 328 - protein, 11 aa, synthetic construct
GNVRTPGCNRQ

SEQ ID NO: 329 - protein, 11 aa, synthetic construct
GNVRSTASTRQ

SEQ ID NO: 330 - protein, 11 aa, synthetic construct
GNVQHTKRGRQ

SEQ ID NO: 331 - protein, 11 aa, synthetic construct
GNVMAGLCARQ

SEQ ID NO: 332 - protein, 11 aa, synthetic construct
GNVLGTVLVRQ

SEQ ID NO: 333 - protein, 11 aa, synthetic construct
GNVLGRMLARQ

SEQ ID NO: 334 - protein, 11 aa, synthetic construct
GNVFSMSTLRQ

SEQ ID NO: 335 - protein, 11 aa, synthetic construct
GNVCISQMNRQ

SEQ ID NO: 336 - protein, 11 aa, synthetic construct
GNVAYCPSQRQ

SEQ ID NO: 337 - protein, 11 aa, synthetic construct
GNTPFRVVLRQ

SEQ ID NO: 338 - protein, 11 aa, synthetic construct
GNTLSHSWTRQ

SEQ ID NO: 339 - protein, 11 aa, synthetic construct
GNTHSTFYPRQ

SEQ ID NO: 340 - protein, 11 aa, synthetic construct
GNTGRRDYQRQ

SEQ ID NO: 341 - protein, 11 aa, synthetic construct
GNTFYSTHVRQ

SEQ ID NO: 342 - protein, 11 aa, synthetic construct
GNTDKRLSTRQ

SEQ ID NO: 343 - protein, 11 aa, synthetic construct
GNTAPLLLLRQ

SEQ ID NO: 344 - protein, 11 aa, synthetic construct
GNSTKRTDSRQ

SEQ ID NO: 345 - protein, 11 aa, synthetic construct
GNSRYAFMIRQ

SEQ ID NO: 346 - protein, 11 aa, synthetic construct
GNSRNASDDRR

SEQ ID NO: 347 - protein, 11 aa, synthetic construct
GNSRLKHGDRQ

SEQ ID NO: 348 - protein, 11 aa, synthetic construct
GNSPTLSTFRR

SEQ ID NO: 349 - protein, 11 aa, synthetic construct
GNSCIMSMRRQ

SEQ ID NO: 350 - protein, 11 aa, synthetic construct
GNSCANTVWRQ

SEQ ID NO: 351 - protein, 11 aa, synthetic construct
GNSASRVILRQ

SEQ ID NO: 352 - protein, 11 aa, synthetic construct
GNRYNFSDSRQ

SEQ ID NO: 353 - protein, 11 aa, synthetic construct
GNRPGALSRRQ

SEQ ID NO: 354 - protein, 11 aa, synthetic construct
GNRNVLPQRRQ

SEQ ID NO: 355 - protein, 11 aa, synthetic construct
GNRISITIGRQ

SEQ ID NO: 356 - protein, 11 aa, synthetic construct
GNRFPTSPLRQ

SEQ ID NO: 357 - protein, 11 aa, synthetic construct
GNRALRMFLRQ

SEQ ID NO: 358 - protein, 11 aa, synthetic construct
GNQPWSVRRRQ

SEQ ID NO: 359 - protein, 11 aa, synthetic construct
GNQMTIIGSRQ

SEQ ID NO: 360 - protein, 11 aa, synthetic construct
GNPVYVRMVRQ

SEQ ID NO: 361 - protein, 11 aa, synthetic construct
GNPPTPEHSRQ

SEQ ID NO: 362 - protein, 11 aa, synthetic construct
GNPMLLVVSRQ

SEQ ID NO: 363 - protein, 11 aa, synthetic construct
GNPLLGSLMRQ

SEQ ID NO: 364 - protein, 11 aa, synthetic construct
GNPLCSSQGRQ

SEQ ID NO: 365 - protein, 11 aa, synthetic construct
GNPCCKYFIRQ

SEQ ID NO: 366 - protein, 11 aa, synthetic construct
GNPALPRNRRQ

SEQ ID NO: 367 - protein, 11 aa, synthetic construct
GNNFLGLISRQ

SEQ ID NO: 368 - protein, 11 aa, synthetic construct
GNNCYPSDLRQ

SEQ ID NO: 369 - protein, 11 aa, synthetic construct
GNNCTIPSLRQ

SEQ ID NO: 370 - protein, 11 aa, synthetic construct
GNMSTSVWSRQ

SEQ ID NO: 371 - protein, 11 aa, synthetic construct
GNMSLTGTTRQ

SEQ ID NO: 372 - protein, 11 aa, synthetic construct
GNMRSPIGQRQ

SEQ ID NO: 373 - protein, 11 aa, synthetic construct
GNMRIMVPWRQ

SEQ ID NO: 374 - protein, 11 aa, synthetic construct
GNMPLCTPTRQ

SEQ ID NO: 375 - protein, 11 aa, synthetic construct
GNMCKPALHRQ

SEQ ID NO: 376 - protein, 11 aa, synthetic construct
GNMACSLGPRQ

SEQ ID NO: 377 - protein, 11 aa, synthetic construct
GNLTYQFPPRQ

SEQ ID NO: 378 - protein, 11 aa, synthetic construct
GNLSPFLQKRQ

SEQ ID NO: 379 - protein, 11 aa, synthetic construct
GNLSLSMTIRQ

SEQ ID NO: 380 - protein, 11 aa, synthetic construct
GNLSLSIKPRQ

SEQ ID NO: 381 - protein, 11 aa, synthetic construct
GNLSLSFPSRQ

SEQ ID NO: 382 - protein, 11 aa, synthetic construct
GNLSGIGFSRQ

SEQ ID NO: 383 - protein, 11 aa, synthetic construct
GNLRYQKAMRQ

SEQ ID NO: 384 - protein, 11 aa, synthetic construct
GNLRFYYPYRQ

SEQ ID NO: 385 - protein, 11 aa, synthetic construct
GNLMTGWTSRQ

SEQ ID NO: 386 - protein, 11 aa, synthetic construct
GNLLSWQDMRQ

SEQ ID NO: 387 - protein, 11 aa, synthetic construct
GNLLDLSMARQ

SEQ ID NO: 388 - protein, 11 aa, synthetic construct
GNLHSKGGARQ

SEQ ID NO: 389 - protein, 11 aa, synthetic construct
GNLHRLMGHRQ

SEQ ID NO: 390 - protein, 11 aa, synthetic construct
GNLHPWQTPRQ

SEQ ID NO: 391 - protein, 11 aa, synthetic construct
GNLEFWSSFRQ

SEQ ID NO: 392 - protein, 11 aa, synthetic construct
GNLCYIYMTRQ

SEQ ID NO: 393 - protein, 11 aa, synthetic construct
GNKEIARVRRQ

SEQ ID NO: 394 - protein, 11 aa, synthetic construct
GNIYILKRHRQ

SEQ ID NO: 395 - protein, 11 aa, synthetic construct
GNIRHGYYDRQ

SEQ ID NO: 396 - protein, 11 aa, synthetic construct
GNINRGLSTRQ

SEQ ID NO: 397 - protein, 11 aa, synthetic construct
GNILRILPSRQ

SEQ ID NO: 398 - protein, 11 aa, synthetic construct
GNILFHSSSRQ

SEQ ID NO: 399 - protein, 11 aa, synthetic construct
GNIKECSTPRQ

SEQ ID NO: 400 - protein, 11 aa, synthetic construct
GNIISWTGTRQ

SEQ ID NO: 401 - protein, 11 aa, synthetic construct
GNIFYSWCHRQ

SEQ ID NO: 402 - protein, 11 aa, synthetic construct
GNIERNGKSRQ

SEQ ID NO: 403 - protein, 11 aa, synthetic construct
GNHVSSRTARQ

SEQ ID NO: 404 - protein, 11 aa, synthetic construct
GNHTDFIYSRQ

SEQ ID NO: 405 - protein, 11 aa, synthetic construct
GNHSHYTIARQ

SEQ ID NO: 406 - protein, 11 aa, synthetic construct
GNGVMQWFQRQ

SEQ ID NO: 407 - protein, 11 aa, synthetic construct
GNGTSMSSRRQ

SEQ ID NO: 408 - protein, 11 aa, synthetic construct
GNGRGPTQQRQ

SEQ ID NO: 409 - protein, 11 aa, synthetic construct
GNGQERFWLRQ

SEQ ID NO: 410 - protein, 11 aa, synthetic construct
GNGPALHSVRR

SEQ ID NO: 411 - protein, 11 aa, synthetic construct
GNGMQACYGRQ

SEQ ID NO: 412 - protein, 11 aa, synthetic construct
GNGMPCFGARQ

SEQ ID NO: 413 - protein, 11 aa, synthetic construct
GNGLLSYTGRQ

SEQ ID NO: 414 - protein, 11 aa, synthetic construct
GNGKSTLFYRQ

SEQ ID NO: 415 - protein, 11 aa, synthetic construct
GNGGFSIYERQ

SEQ ID NO: 416 - protein, 11 aa, synthetic construct
GNGARVWPVRQ

SEQ ID NO: 417 - protein, 11 aa, synthetic construct
GNFYLKGTSRQ

SEQ ID NO: 418 - protein, 11 aa, synthetic construct
GNFTRHQPSRQ

SEQ ID NO: 419 - protein, 11 aa, synthetic construct
GNFHDLRIIRQ

SEQ ID NO: 420 - protein, 11 aa, synthetic construct
GNFFNIPFSRQ

SEQ ID NO: 421 - protein, 11 aa, synthetic construct
GNELFHVKKRQ

SEQ ID NO: 422 - protein, 11 aa, synthetic construct
GNDWTHHSSRQ

SEQ ID NO: 423 - protein, 11 aa, synthetic construct
GNDPNCQRNRQ

SEQ ID NO: 424 - protein, 11 aa, synthetic construct
GNDITTCNYRQ

SEQ ID NO: 425 - protein, 11 aa, synthetic construct
GNDCSPFPPRQ

SEQ ID NO: 426 - protein, 11 aa, synthetic construct
GNCWVTRIQRQ

SEQ ID NO: 427 - protein, 11 aa, synthetic construct
GNCVRSQNTRQ

SEQ ID NO: 428 - protein, 11 aa, synthetic construct
GNCQALGIMRQ

SEQ ID NO: 429 - protein, 11 aa, synthetic construct
GNCLSRSYIRQ

SEQ ID NO: 430 - protein, 11 aa, synthetic construct
GNCLGDPRLRQ

SEQ ID NO: 431 - protein, 11 aa, synthetic construct
GNAYLLHTPRQ

SEQ ID NO: 432 - protein, 11 aa, synthetic construct
GNARQCVVDRQ

SEQ ID NO: 433 - protein, 11 aa, synthetic construct
GNAMPFMPGRQ

SEQ ID NO: 434 - protein, 11 aa, synthetic construct
GNADYLLVLRQ

SEQ ID NO: 435 - protein, 11 aa, synthetic construct
GIKVCLCMPRQ

SEQ ID NO: 436 - protein, 11 aa, synthetic construct
CNTSPVGTLRQ

SEQ ID NO: 437 - protein, 11 aa, synthetic construct
CNSRLKLGDRQ

SEQ ID NO: 438 - protein, 11 aa, synthetic construct
GNGNRVTAPRQ

SEQ ID NO: 439 - protein, 11 aa, synthetic construct
GNNHASEPLRQ

SEQ ID NO: 440 - protein, 11 aa, synthetic construct
GNNRTGLPIRQ

SEQ ID NO: 441 - protein, 11 aa, synthetic construct
GNENRIERQRQ

SEQ ID NO: 442 - protein, 11 aa, synthetic construct
GNLSRQSVSRQ

SEQ ID NO: 443 - protein, 11 aa, synthetic construct
GNVKPTGSPRQ

SEQ ID NO: 444 - protein, 11 aa, synthetic construct
GNASRSILARQ

SEQ ID NO: 445 - protein, 11 aa, synthetic construct
GNQSRTSTPRQ

SEQ ID NO: 446 - protein, 11 aa, synthetic construct
GNSTQRLQARQ

SEQ ID NO: 447 - protein, 11 aa, synthetic construct
GNNSSKNLTRQ

SEQ ID NO: 448 - protein, 11 aa, synthetic construct
GNSSRMTSPRQ

SEQ ID NO: 449 - protein, 11 aa, synthetic construct
GNLTSNKLTRQ

SEQ ID NO: 450 - protein, 11 aa, synthetic construct
GNPNSAKAERQ

SEQ ID NO: 451 - protein, 11 aa, synthetic construct
GNNTTKSPARQ

SEQ ID NO: 452 - protein, 11 aa, synthetic construct
GNNVTKIPSRQ

SEQ ID NO: 453 - protein, 11 aa, synthetic construct
GNMNSARADRQ

SEQ ID NO: 454 - protein, 11 aa, synthetic construct
GKSNQTAPSRQ

SEQ ID NO: 455 - protein, 11 aa, synthetic construct
GNNSRQQTPRQ

SEQ ID NO: 456 - protein, 11 aa, synthetic construct
GNSNEVRQGRQ

SEQ ID NO: 457 - protein, 11 aa, synthetic construct
GNNTARLQIRQ

SEQ ID NO: 458 - protein, 11 aa, synthetic construct
GNSGPSSDPRQ

SEQ ID NO: 459 - protein, 11 aa, synthetic construct
GNMVKALSQRQ

SEQ ID NO: 460 - protein, 11 aa, synthetic construct
GNTTKAPSTRQ

SEQ ID NO: 461 - protein, 11 aa, synthetic construct
GNTPTPQHQRQ

SEQ ID NO: 462 - protein, 11 aa, synthetic construct
GNAQNRSPSRQ

SEQ ID NO: 463 - protein, 11 aa, synthetic construct
GNAGKAQMARQ

SEQ ID NO: 464 - protein, 11 aa, synthetic construct
GNATRQPPVRQ

SEQ ID NO: 465 - protein, 11 aa, synthetic construct
GNVLPGLNARL

SEQ ID NO: 466 - protein, 11 aa, synthetic construct
GNSVKMSPNRQ

SEQ ID NO: 467 - protein, 11 aa, synthetic construct
GNTSKTIQPRQ

SEQ ID NO: 468 - protein, 11 aa, synthetic construct
GNETKRSSERQ

SEQ ID NO: 469 - protein, 11 aa, synthetic construct
GNIRSIPPPRQ

SEQ ID NO: 470 - protein, 11 aa, synthetic construct
GNQNSVRQDRQ

SEQ ID NO: 471 - protein, 11 aa, synthetic construct
GNNLEQLRARQ

SEQ ID NO: 472 - protein, 11 aa, synthetic construct
GNSGNLGRERQ

SEQ ID NO: 473 - protein, 11 aa, synthetic construct
GNSRSTIPARQ

SEQ ID NO: 474 - protein, 11 aa, synthetic construct
GNTSRQVSPRQ

SEQ ID NO: 475 - protein, 11 aa, synthetic construct
GNTNKIVPSRQ

SEQ ID NO: 476 - protein, 11 aa, synthetic construct
GNSIKQPGQRQ

SEQ ID NO: 477 - protein, 11 aa, synthetic construct
GNSTRGIVARQ

SEQ ID NO: 478 - protein, 11 aa, synthetic construct
GNSIKSAQYRQ

SEQ ID NO: 479 - protein, 11 aa, synthetic construct
GNTTRIAQPRQ

SEQ ID NO: 480 - protein, 11 aa, synthetic construct
GNTIRVGGPRQ

SEQ ID NO: 481 - protein, 11 aa, synthetic construct
GNSVRSMTARQ

SEQ ID NO: 482 - protein, 11 aa, synthetic construct
GNLATKIPPRQ

SEQ ID NO: 483 - protein, 11 aa, synthetic construct
GNNVTKIPARL

SEQ ID NO: 484 - protein, 11 aa, synthetic construct
GNTLPIGAQRQ

SEQ ID NO: 485 - protein, 11 aa, synthetic construct
GNLGASKLPRQ

SEQ ID NO: 486 - protein, 11 aa, synthetic construct
GNQSKAGMSRQ

SEQ ID NO: 487 - protein, 11 aa, synthetic construct
GNFTRQEPTRQ

SEQ ID NO: 488 - protein, 11 aa, synthetic construct
GNDNTSTRSRQ

SEQ ID NO: 489 - protein, 11 aa, synthetic construct
GNTLSNKVSRQ

SEQ ID NO: 490 - protein, 11 aa, synthetic construct
GNMNTSRQDRQ

SEQ ID NO: 491 - protein, 11 aa, synthetic construct
GNLGHQKPARQ

SEQ ID NO: 492 - protein, 11 aa, synthetic construct
GNIVPGLNQRQ

SEQ ID NO: 493 - protein, 11 aa, synthetic construct
GNPPTHTQDRQ

SEQ ID NO: 494 - protein, 11 aa, synthetic construct
GNMTNRTQIRQ

SEQ ID NO: 495 - protein, 11 aa, synthetic construct
GNTSRAQSPRQ

SEQ ID NO: 496 - protein, 11 aa, synthetic construct
GNTNSAKGDRQ

SEQ ID NO: 497 - protein, 11 aa, synthetic construct
GNQAPLKQSRQ

SEQ ID NO: 498 - protein, 11 aa, synthetic construct
GNNVTKITARQ

SEQ ID NO: 499 - protein, 11 aa, synthetic construct
GNFGVTRSPRQ

SEQ ID NO: 500 - protein, 11 aa, synthetic construct
GNNASKSPVRQ

SEQ ID NO: 501 - protein, 11 aa, synthetic construct
GNVNTVRGDRQ

SEQ ID NO: 502 - protein, 11 aa, synthetic construct
GNLNETSRTRQ

SEQ ID NO: 503 - protein, 11 aa, synthetic construct
GNETRARLERQ

SEQ ID NO: 504 - protein, 11 aa, synthetic construct
GNNVTKIPARQ

SEQ ID NO: 505 - protein, 11 aa, synthetic construct
GNNLTKIPARQ

SEQ ID NO: 506 - protein, 11 aa, synthetic construct
GNSTKATSPRQ

SEQ ID NO: 507 - protein, 11 aa, synthetic construct
GNNVSKIPARQ

SEQ ID NO: 508 - protein, 11 aa, synthetic construct
GNNVIKSSVRQ

SEQ ID NO: 509 - protein, 11 aa, synthetic construct
GNNRTSPMVRQ

SEQ ID NO: 510 - protein, 11 aa, synthetic construct
GNNQSKLNVRQ

SEQ ID NO: 511 - protein, 11 aa, synthetic construct
GNNVTKNPARQ

SEQ ID NO: 512 - protein, 11 aa, synthetic construct
GNNVTKIQARQ

SEQ ID NO: 513 - protein, 11 aa, synthetic construct
GNSVKLPATRQ

SEQ ID NO: 514 - protein, 11 aa, synthetic construct
GNSIKSPGFRQ

SEQ ID NO: 515 - protein, 11 aa, synthetic construct
GNNAVKIPPRQ

SEQ ID NO: 516 - protein, 11 aa, synthetic construct
GNYNQTSRDRQ

SEQ ID NO: 517 - protein, 11 aa, synthetic construct
GNQENVIRSRQ

SEQ ID NO: 518 - protein, 11 aa, synthetic construct
GNSKPQSSPRQ

SEQ ID NO: 519 - protein, 11 aa, synthetic construct
GNRPDARLARQ

SEQ ID NO: 520 - protein, 11 aa, synthetic construct
GNNVTKIPPRQ

SEQ ID NO: 521 - protein, 11 aa, synthetic construct
GNVDKAKLARQ

SEQ ID NO: 522 - protein, 11 aa, synthetic construct
GNLVPGIQARQ

SEQ ID NO: 523 - protein, 11 aa, synthetic construct
GNRTTTIPPRQ

SEQ ID NO: 524 - protein, 11 aa, synthetic construct
GNMSNLPPPRQ

SEQ ID NO: 525 - protein, 11 aa, synthetic construct
GNENRIRQERQ

SEQ ID NO: 526 - protein, 11 aa, synthetic construct
GNFNDTRLSRQ

SEQ ID NO: 527 - protein, 11 aa, synthetic construct
GNTNRTQSVRQ

SEQ ID NO: 528 - protein, 11 aa, synthetic construct
GNNSDTGRPRQ

SEQ ID NO: 529 - protein, 11 aa, synthetic construct
GNNFTKIPARQ

SEQ ID NO: 530 - protein, 11 aa, synthetic construct
GNNIQRIPARQ

SEQ ID NO: 531 - protein, 11 aa, synthetic construct
GNIVAGLDQRQ

SEQ ID NO: 532 - protein, 11 aa, synthetic construct
GNVVSGLDARQ

SEQ ID NO: 533 - protein, 11 aa, synthetic construct
GNVSAYMHARQ

SEQ ID NO: 534 - protein, 11 aa, synthetic construct
GNNTAKALPRQ

SEQ ID NO: 535 - protein, 11 aa, synthetic construct
GNSIKVVQTRQ

SEQ ID NO: 536 - protein, 11 aa, synthetic construct
GNNYRTGTPRQ

SEQ ID NO: 537 - protein, 11 aa, synthetic construct
GNSVVRSSPRQ

SEQ ID NO: 538 - protein, 11 aa, synthetic construct
GNSNKTAPSRQ

SEQ ID NO: 539 - protein, 11 aa, synthetic construct
GNSTKRTDTRQ

SEQ ID NO: 540 - protein, 11 aa, synthetic construct
GNGTRGLSPRQ

SEQ ID NO: 541 - protein, 11 aa, synthetic construct
GNSNQRILPRQ

SEQ ID NO: 542 - protein, 11 aa, synthetic construct
GNSTKAPQPRQ

SEQ ID NO: 543 - protein, 11 aa, synthetic construct
GNRTSVGSPRQ

SEQ ID NO: 544 - protein, 11 aa, synthetic construct
GNTLSKIPARQ

SEQ ID NO: 545 - protein, 11 aa, synthetic construct
GNAAPLRLSRQ

SEQ ID NO: 546 - protein, 11 aa, synthetic construct
GNAGVKTTPRQ

SEQ ID NO: 547 - protein, 11 aa, synthetic construct
GNTGSLESLRQ

SEQ ID NO: 548 - protein, 11 aa, synthetic construct
GNLGKVGSPRQ

SEQ ID NO: 549 - protein, 11 aa, synthetic construct
GNIPTLSAQRQ

SEQ ID NO: 550 - protein, 11 aa, synthetic construct
GNYFGSGRERQ

SEQ ID NO: 551 - protein, 11 aa, synthetic construct
GNAGKSQMARQ

SEQ ID NO: 552 - protein, 11 aa, synthetic construct
GNVVPGLSYLQ

SEQ ID NO: 553 - protein, 11 aa, synthetic construct
GNQTRTNLPRQ

SEQ ID NO: 554 - protein, 11 aa, synthetic construct
GNKPPTHSPRQ

SEQ ID NO: 555 - protein, 11 aa, synthetic construct
GNSTKDKNFRQ

SEQ ID NO: 556 - protein, 11 aa, synthetic construct
GNLVKSLQSRQ

SEQ ID NO: 557 - protein, 11 aa, synthetic construct
GNSVRLTSPRQ

SEQ ID NO: 558 - protein, 11 aa, synthetic construct
GNINGQKLSRQ

SEQ ID NO: 559 - protein, 11 aa, synthetic construct
GNSDKRASPRQ

SEQ ID NO: 560 - protein, 11 aa, synthetic construct
GNLRPTGAPRQ

SEQ ID NO: 561 - protein, 11 aa, synthetic construct
GNFTVSPPPRQ

SEQ ID NO: 562 - protein, 11 aa, synthetic construct
GNSTKSQQMRQ

SEQ ID NO: 563 - protein, 11 aa, synthetic construct
GNMNHIRNERQ

SEQ ID NO: 564 - protein, 11 aa, synthetic construct
GNNQIKLGMRQ

SEQ ID NO: 565 - protein, 11 aa, synthetic construct
GNLVPGLSYRQ

SEQ ID NO: 566 - protein, 11 aa, synthetic construct
GNSTKKDSIRQ

SEQ ID NO: 567 - protein, 11 aa, synthetic construct
GNVLKNTTARQ

SEQ ID NO: 568 - protein, 11 aa, synthetic construct
GNLVESLRARQ

SEQ ID NO: 569 - protein, 11 aa, synthetic construct
GNTNQTRSERQ

SEQ ID NO: 570 - protein, 11 aa, synthetic construct
GNSANVVLRRQ

SEQ ID NO: 571 - protein, 11 aa, synthetic construct
GNTLGKMQSRQ

SEQ ID NO: 572 - protein, 11 aa, synthetic construct
GNLVPGIPARL

SEQ ID NO: 573 - protein, 11 aa, synthetic construct
GNLTKALQGRQ

SEQ ID NO: 574 - protein, 11 aa, synthetic construct
GNTPAFKTPRQ

SEQ ID NO: 575 - protein, 11 aa, synthetic construct
GNIGSGSEHRQ

SEQ ID NO: 576 - protein, 11 aa, synthetic construct
GNVMRNTIARQ

SEQ ID NO: 577 - protein, 11 aa, synthetic construct
GNLNKTQGFRQ

SEQ ID NO: 578 - protein, 11 aa, synthetic construct
GNPDISGQTRQ

SEQ ID NO: 579 - protein, 11 aa, synthetic construct
GNSDRAKSPRQ

SEQ ID NO: 580 - protein, 11 aa, synthetic construct
GNATKIIQSRQ

SEQ ID NO: 581 - protein, 11 aa, synthetic construct
GNSSRQQPSRQ

SEQ ID NO: 582 - protein, 11 aa, synthetic construct
GNTITNRAERQ

SEQ ID NO: 583 - protein, 11 aa, synthetic construct
GNSSSNLMKRQ

SEQ ID NO: 584 - protein, 11 aa, synthetic construct
GNVNQSRSDRQ

SEQ ID NO: 585 - protein, 11 aa, synthetic construct
GNSNQVQRDRQ

SEQ ID NO: 586 - protein, 11 aa, synthetic construct
GNGRPTSLPRQ

SEQ ID NO: 587 - protein, 11 aa, synthetic construct
GNLTQRSPSRQ

SEQ ID NO: 588 - protein, 11 aa, synthetic construct
GNSDNAVTVRQ

SEQ ID NO: 589 - protein, 11 aa, synthetic construct
GNVLRKLPERQ

SEQ ID NO: 590 - protein, 11 aa, synthetic construct
GNSRVGLNPRQ

SEQ ID NO: 591 - protein, 11 aa, synthetic construct
GNTTSPLKARQ

SEQ ID NO: 592 - protein, 11 aa, synthetic construct
GNNAAKAMIRQ

SEQ ID NO: 593 - protein, 11 aa, synthetic construct
GNSTKVVLSRQ

SEQ ID NO: 594 - protein, 11 aa, synthetic construct
GNIVAGLTSRL

SEQ ID NO: 595 - protein, 11 aa, synthetic construct
GNPMRALQPRQ

SEQ ID NO: 596 - protein, 11 aa, synthetic construct
GNDNATLRPRQ

SEQ ID NO: 597 - protein, 11 aa, synthetic construct
GNNVTKILARQ

SEQ ID NO: 598 - protein, 11 aa, synthetic construct
GNATKIIQTRQ

SEQ ID NO: 599 - protein, 11 aa, synthetic construct
GNNNRSLPIRQ

SEQ ID NO: 600 - protein, 11 aa, synthetic construct
GNASRLGTARQ

SEQ ID NO: 601 - protein, 11 aa, synthetic construct
GNQVRTISPRQ

SEQ ID NO: 602 - protein, 11 aa, synthetic construct
GNLLRNTTARQ

SEQ ID NO: 603 - protein, 11 aa, synthetic construct
GNTTQRLQARQ

SEQ ID NO: 604 - protein, 11 aa, synthetic construct
GNLIKNATSRQ

SEQ ID NO: 605 - protein, 11 aa, synthetic construct
GNQNRTLPLRQ

SEQ ID NO: 606 - protein, 11 aa, synthetic construct
GNITKNTTVRQ

SEQ ID NO: 607 - protein, 11 aa, synthetic construct
GNSTQRLQVRQ

SEQ ID NO: 608 - protein, 11 aa, synthetic construct
GNNTRGRDDRQ

SEQ ID NO: 609 - protein, 11 aa, synthetic construct
GNRTKVSDPRQ

SEQ ID NO: 610 - protein, 11 aa, synthetic construct
GNLGIPKSPRQ

SEQ ID NO: 611 - protein, 11 aa, synthetic construct
GNVTPYKTGRQ

SEQ ID NO: 612 - protein, 11 aa, synthetic construct
GNVQKPHPPRQ

SEQ ID NO: 613 - protein, 11 aa, synthetic construct
GNTTRSSIPRQ

SEQ ID NO: 614 - protein, 11 aa, synthetic construct
GNTTRIPPSRQ

SEQ ID NO: 615 - protein, 11 aa, synthetic construct
GNTTRGIPSRQ

SEQ ID NO: 616 - protein, 11 aa, synthetic construct
GNTPQVRMSRQ

SEQ ID NO: 617 - protein, 11 aa, synthetic construct
GNSTQRLQSRQ

SEQ ID NO: 618 - protein, 11 aa, synthetic construct
GNSPQPSRARQ

SEQ ID NO: 619 - protein, 11 aa, synthetic construct
GNSLRDRSMRQ

SEQ ID NO: 620 - protein, 11 aa, synthetic construct
GNSIVRSAVRQ

SEQ ID NO: 621 - protein, 11 aa, synthetic construct
GNSGQTPRPRQ

SEQ ID NO: 622 - protein, 11 aa, synthetic construct
GNSGPMKTSRQ

SEQ ID NO: 623 - protein, 11 aa, synthetic construct
GNRTSQPPPRQ

SEQ ID NO: 624 - protein, 11 aa, synthetic construct
GNRGRPSDPRQ

SEQ ID NO: 625 - protein, 11 aa, synthetic construct
GNQRTYAPPRQ

SEQ ID NO: 626 - protein, 11 aa, synthetic construct
GNQIRTMGPRQ

SEQ ID NO: 627 - protein, 11 aa, synthetic construct
GNQIRLGGSRQ

SEQ ID NO: 628 - protein, 11 aa, synthetic construct
GNNVTAKAPRQ

SEQ ID NO: 629 - protein, 11 aa, synthetic construct
GNNSVPRSPRQ

SEQ ID NO: 630 - protein, 11 aa, synthetic construct
GNNMSRTISRQ

SEQ ID NO: 631 - protein, 11 aa, synthetic construct
GNLTRQAGSRQ

SEQ ID NO: 632 - protein, 11 aa, synthetic construct
GNLEQSGRERR

SEQ ID NO: 633 - protein, 11 aa, synthetic construct
GNISDSGRPRQ

SEQ ID NO: 634 - protein, 11 aa, synthetic construct
GNIKVSPPPRQ

SEQ ID NO: 635 - protein, 11 aa, synthetic construct
GNHFPSVLLRQ

SEQ ID NO: 636 - protein, 11 aa, synthetic construct
GNASNRLTARQ

SEQ ID NO: 637 - protein, 11 aa, synthetic construct
GNANRSQSPRQ

SEQ ID NO: 638 - protein, 11 aa, synthetic construct
GNAISNLAKRQ

SEQ ID NO: 639 - protein, 11 aa, synthetic construct
GNVYTKLWMRQ

SEQ ID NO: 640 - protein, 11 aa, synthetic construct
GNCLSSSLNRQ

SEQ ID NO: 641 - protein, 11 aa, synthetic construct
GNRAHFLWNRQ

SEQ ID NO: 642 - protein, 11 aa, synthetic construct
GNAPDFSYPRQ

SEQ ID NO: 643 - protein, 11 aa, synthetic construct
GNRPRVITLRQ

SEQ ID NO: 644 - protein, 11 aa, synthetic construct
GNMVYNLQMRQ

SEQ ID NO: 645 - protein, 11 aa, synthetic construct
GNSHVSKRSRQ

SEQ ID NO: 646 - protein, 11 aa, synthetic construct
GNSAIFTGPRQ

SEQ ID NO: 647 - protein, 11 aa, synthetic construct
GNSIQQGPRRQ

SEQ ID NO: 648 - protein, 11 aa, synthetic construct
CNERSSQSSRQ

SEQ ID NO: 649 - protein, 11 aa, synthetic construct
GNLQSFAIQRQ

SEQ ID NO: 650 - protein, 11 aa, synthetic construct
GNLTETVESRQ

SEQ ID NO: 651 - protein, 11 aa, synthetic construct
GNLDYYSSTRQ

SEQ ID NO: 652 - protein, 11 aa, synthetic construct
GNISIMFGSRQ

SEQ ID NO: 653 - protein, 11 aa, synthetic construct
GNVKRLMLLRQ

SEQ ID NO: 654 - protein, 11 aa, synthetic construct
GNYLNEFMARL

SEQ ID NO: 655 - protein, 11 aa, synthetic construct
GNILGSILTRQ

SEQ ID NO: 656 - protein, 11 aa, synthetic construct
GNRVMGSKLRQ

SEQ ID NO: 657 - protein, 11 aa, synthetic construct
GNIPRLTDVRL

SEQ ID NO: 658 - protein, 11 aa, synthetic construct
GNHHRSIHHRQ

SEQ ID NO: 659 - protein, 11 aa, synthetic construct
GNDISSVMSRQ

SEQ ID NO: 660 - protein, 11 aa, synthetic construct
GNCEGWYGRRQ

SEQ ID NO: 661 - protein, 11 aa, synthetic construct
GNRWAPLNQRQ

SEQ ID NO: 662 - protein, 11 aa, synthetic construct
GNPQSGSPERQ

SEQ ID NO: 663 - protein, 11 aa, synthetic construct
GNQVSGQYQRQ

SEQ ID NO: 664 - protein, 11 aa, synthetic construct
GNDLLLISSRQ

SEQ ID NO: 665 - protein, 11 aa, synthetic construct
GNGPVLMSSRQ

SEQ ID NO: 666 - protein, 11 aa, synthetic construct
GNCYNYMLVRQ

SEQ ID NO: 667 - protein, 11 aa, synthetic construct
GNTMPHIRLRQ

SEQ ID NO: 668 - protein, 11 aa, synthetic construct
GNSFNRRTLRQ

SEQ ID NO: 669 - protein, 11 aa, synthetic construct
GNFHNFPSPRQ

SEQ ID NO: 670 - protein, 11 aa, synthetic construct
GNSLQALEPRQ

SEQ ID NO: 671 - protein, 11 aa, synthetic construct
GNEGVCILARQ

SEQ ID NO: 672 - protein, 11 aa, synthetic construct
GNLRDQKAKRQ

SEQ ID NO: 673 - protein, 11 aa, synthetic construct
GNPQASVLRRQ

SEQ ID NO: 674 - protein, 11 aa, synthetic construct
GNNGHGYYDRQ

SEQ ID NO: 675 - protein, 11 aa, synthetic construct
GNHMTSMPSRR

SEQ ID NO: 676 - protein, 11 aa, synthetic construct
GNSQWHSSMRQ

SEQ ID NO: 677 - protein, 11 aa, synthetic construct
GNSQDSMQTRQ

SEQ ID NO: 678 - protein, 11 aa, synthetic construct
GNWYLWIRIRQ

SEQ ID NO: 679 - protein, 11 aa, synthetic construct
GNGYSLTTSRQ

SEQ ID NO: 680 - protein, 11 aa, synthetic construct
GNGVGHVHARQ

SEQ ID NO: 681 - protein, 11 aa, synthetic construct
GNPWQLQVNRQ

SEQ ID NO: 682 - protein, 11 aa, synthetic construct
GNQYSLAASRQ

SEQ ID NO: 683 - protein, 11 aa, synthetic construct
GSAMPTSQERQ

SEQ ID NO: 684 - protein, 11 aa, synthetic construct
GNLGHYQPYRQ

SEQ ID NO: 685 - protein, 11 aa, synthetic construct
GNSRLKLRDRQ

SEQ ID NO: 686 - protein, 11 aa, synthetic construct
GNAMYSSSFRQ

SEQ ID NO: 687 - protein, 11 aa, synthetic construct
GNVRSSATTRQ

SEQ ID NO: 688 - protein, 11 aa, synthetic construct
GNGPALHSLRQ

SEQ ID NO: 689 - protein, 11 aa, synthetic construct
GNTGDSGPLRQ

SEQ ID NO: 690 - protein, 11 aa, synthetic construct
GNSVTFGSPRQ

SEQ ID NO: 691 - protein, 11 aa, synthetic construct
GNTVLKRTLRQ

SEQ ID NO: 692 - protein, 11 aa, synthetic construct
GNNPQDTLGRQ

SEQ ID NO: 693 - protein, 11 aa, synthetic construct
GNSSTLKPGRQ

SEQ ID NO: 694 - protein, 11 aa, synthetic construct
GNPLKQEVPRQ

SEQ ID NO: 695 - protein, 11 aa, synthetic construct
GNPLPTSPLRQ

SEQ ID NO: 696 - protein, 11 aa, synthetic construct
GNAGSLGLKLQ

SEQ ID NO: 697 - protein, 11 aa, synthetic construct
GNKGTPAEERQ

SEQ ID NO: 698 - protein, 11 aa, synthetic construct
GNQEGVGMSRL

SEQ ID NO: 699 - protein, 11 aa, synthetic construct
GNSRSPSTQRQ

SEQ ID NO: 700 - protein, 11 aa, synthetic construct
GNEDSPSQMRQ

SEQ ID NO: 701 - protein, 11 aa, synthetic construct
GNISTLQPIRQ

SEQ ID NO: 702 - protein, 11 aa, synthetic construct
GNALTLSYVRQ

SEQ ID NO: 703 - protein, 11 aa, synthetic construct
GNVVSLQFERQ

SEQ ID NO: 704 - protein, 11 aa, synthetic construct
GNSPSTPILRQ

SEQ ID NO: 705 - protein, 11 aa, synthetic construct
GNDEPPQMSRQ

SEQ ID NO: 706 - protein, 11 aa, synthetic construct
GNIDNAEPRRQ

SEQ ID NO: 707 - protein, 11 aa, synthetic construct
CNTESTAMSRQ

SEQ ID NO: 708 - protein, 11 aa, synthetic construct
GNKLYAITVRQ

SEQ ID NO: 709 - protein, 11 aa, synthetic construct
GNLPSNLSERQ

SEQ ID NO: 710 - protein, 11 aa, synthetic construct
GNHAAPGMCRQ

SEQ ID NO: 711 - protein, 11 aa, synthetic construct
GNLSLIATSRQ

SEQ ID NO: 712 - protein, 11 aa, synthetic construct
GNTQTDSAGRQ

SEQ ID NO: 713 - protein, 11 aa, synthetic construct
GNKVNTRVNRQ

SEQ ID NO: 714 - protein, 11 aa, synthetic construct
GNYPLSPETRQ

SEQ ID NO: 715 - protein, 11 aa, synthetic construct
GNTHSTMYPRQ

SEQ ID NO: 716 - protein, 11 aa, synthetic construct
GNAVVFSDSRQ

SEQ ID NO: 717 - protein, 11 aa, synthetic construct
GNPDGPQHSRQ

SEQ ID NO: 718 - protein, 11 aa, synthetic construct
GNRYRAGPQRQ

SEQ ID NO: 719 - protein, 11 aa, synthetic construct
GNTQDVMNLRQ

SEQ ID NO: 720 - protein, 11 aa, synthetic construct
GNETKLSSERQ

SEQ ID NO: 721 - protein, 11 aa, synthetic construct
GNAYRGFAHRQ

SEQ ID NO: 722 - protein, 11 aa, synthetic construct
GNVLGTVLERQ

SEQ ID NO: 723 - protein, 11 aa, synthetic construct
GNTSQEQGSRQ

SEQ ID NO: 724 - protein, 11 aa, synthetic construct
GNLPPNILSRQ

SEQ ID NO: 725 - protein, 11 aa, synthetic construct
GNAHCDTYQRQ

SEQ ID NO: 726 - protein, 11 aa, synthetic construct
GNKPTHKSARQ

SEQ ID NO: 727 - protein, 11 aa, synthetic construct
GNSDSGLLPRQ

SEQ ID NO: 728 - protein, 11 aa, synthetic construct
GNSLGVALVRQ

SEQ ID NO: 729 - protein, 11 aa, synthetic construct
GNERSALFQRQ

SEQ ID NO: 730 - protein, 11 aa, synthetic construct
GNRPRPPNPRQ

SEQ ID NO: 731 - protein, 11 aa, synthetic construct
GNAMQEGLDRQ

SEQ ID NO: 732 - protein, 11 aa, synthetic construct
GNCPAQIIPRL

SEQ ID NO: 733 - protein, 11 aa, synthetic construct
GNMMQYGYERQ

SEQ ID NO: 734 - protein, 11 aa, synthetic construct
GSVLGHMLARQ

SEQ ID NO: 735 - protein, 11 aa, synthetic construct
GNRAPPLTGRL

SEQ ID NO: 736 - protein, 11 aa, synthetic construct
GNLSERGDSRQ

SEQ ID NO: 737 - protein, 11 aa, synthetic construct
GNPYTGDHPRQ

SEQ ID NO: 738 - protein, 11 aa, synthetic construct
GNINERLEVRQ

SEQ ID NO: 739 - protein, 11 aa, synthetic construct
GNVFSGLAARQ

SEQ ID NO: 740 - protein, 11 aa, synthetic construct
CNQNTAVPTRQ

SEQ ID NO: 741 - protein, 11 aa, synthetic construct
GNIDAHTLGRQ

SEQ ID NO: 742 - protein, 11 aa, synthetic construct
GNSLGVAQERQ

SEQ ID NO: 743 - protein, 11 aa, synthetic construct
GNLGHESLGRQ

SEQ ID NO: 744 - protein, 11 aa, synthetic construct
GNVLGHMLARH

SEQ ID NO: 745 - protein, 11 aa, synthetic construct
GNMLDLHSPRQ

SEQ ID NO: 746 - protein, 11 aa, synthetic construct
GNGLYCTDARQ

SEQ ID NO: 747 - protein, 11 aa, synthetic construct
GNLSLATWVRQ

SEQ ID NO: 748 - protein, 11 aa, synthetic construct
GNHSHLTPERQ

SEQ ID NO: 749 - protein, 11 aa, synthetic construct
GNVHQAIEIRQ

SEQ ID NO: 750 - protein, 11 aa, synthetic construct
GNVTGIHPSRQ

SEQ ID NO: 751 - protein, 11 aa, synthetic construct
GNETAYMHARQ

SEQ ID NO: 752 - protein, 11 aa, synthetic construct
GNILPTSSLRQ

SEQ ID NO: 753 - protein, 11 aa, synthetic construct
GNPSRMMVSRQ

SEQ ID NO: 754 - protein, 11 aa, synthetic construct
GNPSDSGASRQ

SEQ ID NO: 755 - protein, 11 aa, synthetic construct
GNMLGSCPSRQ

SEQ ID NO: 756 - protein, 11 aa, synthetic construct
GNFCMIFVHRQ

SEQ ID NO: 757 - protein, 11 aa, synthetic construct
GNLLCTALTRQ

SEQ ID NO: 758 - protein, 11 aa, synthetic construct
GNNLTVSVQRQ

SEQ ID NO: 759 - protein, 11 aa, synthetic construct
GNNSHRETSRQ

SEQ ID NO: 760 - protein, 11 aa, synthetic construct
GNDIRGPVIRQ

SEQ ID NO: 761 - protein, 11 aa, synthetic construct
GNLRMSAFQRQ

SEQ ID NO: 762 - protein, 11 aa, synthetic construct
GNSYYQTTQRQ

SEQ ID NO: 763 - protein, 11 aa, synthetic construct
GNTIPASLMRQ

SEQ ID NO: 764 - protein, 11 aa, synthetic construct
GNCGITGSARQ

SEQ ID NO: 765 - protein, 11 aa, synthetic construct
GNISTIFMPRQ

SEQ ID NO: 766 - protein, 11 aa, synthetic construct
GNVYHPQLPRQ

SEQ ID NO: 767 - protein, 11 aa, synthetic construct
GNASPLHPQRQ

SEQ ID NO: 768 - protein, 11 aa, synthetic construct
GNQNGPLLLRQ

SEQ ID NO: 769 - protein, 11 aa, synthetic construct
GNSLGTQMVRQ

SEQ ID NO: 770 - protein, 11 aa, synthetic construct
GNRVFHIPMRQ

SEQ ID NO: 771 - protein, 11 aa, synthetic construct
GNYVGTEPARQ

SEQ ID NO: 772 - protein, 11 aa, synthetic construct
GNVLGHMLVRQ

SEQ ID NO: 773 - protein, 11 aa, synthetic construct
GNLEQSGRERQ

SEQ ID NO: 774 - protein, 11 aa, synthetic construct
GNPDTSGSPRQ

SEQ ID NO: 775 - protein, 11 aa, synthetic construct
GNMTPPVSSRQ

SEQ ID NO: 776 - protein, 11 aa, synthetic construct
GNTCAILNTRQ

SEQ ID NO: 777 - protein, 11 aa, synthetic construct
GNQMHASPSRQ

SEQ ID NO: 778 - protein, 11 aa, synthetic construct
GNSSLNLDPRQ

SEQ ID NO: 779 - protein, 11 aa, synthetic construct
GNSLSQSADRQ

SEQ ID NO: 780 - protein, 11 aa, synthetic construct
GNLSAPMYTRQ

SEQ ID NO: 781 - protein, 11 aa, synthetic construct
GNPLPYGSPRQ

SEQ ID NO: 782 - protein, 11 aa, synthetic construct
GNPNEIRLERQ

SEQ ID NO: 783 - protein, 11 aa, synthetic construct
GNPALGVDFRQ

SEQ ID NO: 784 - protein, 11 aa, synthetic construct
GNTRGPNSTRQ

SEQ ID NO: 785 - protein, 11 aa, synthetic construct
GNRLGYQSVRQ

SEQ ID NO: 786 - protein, 11 aa, synthetic construct
GNTLVSTTERQ

SEQ ID NO: 787 - protein, 11 aa, synthetic construct
GNALQPQLSRQ

SEQ ID NO: 788 - protein, 11 aa, synthetic construct
GNTFSSGSERQ

SEQ ID NO: 789 - protein, 11 aa, synthetic construct
GNLSQYILSRQ

SEQ ID NO: 790 - protein, 11 aa, synthetic construct
GIPSPQDFSRQ

SEQ ID NO: 791 - protein, 11 aa, synthetic construct
GNKLSAISVRQ

SEQ ID NO: 792 - protein, 11 aa, synthetic construct
GNTSVLTLTRQ

SEQ ID NO: 793 - protein, 11 aa, synthetic construct
GNVYTKLRKRQ

SEQ ID NO: 794 - protein, 11 aa, synthetic construct
GNNQTSIPLRQ

SEQ ID NO: 795 - protein, 11 aa, synthetic construct
GNRMPSTQTRQ

SEQ ID NO: 796 - protein, 11 aa, synthetic construct
GNGSSPSATRQ

SEQ ID NO: 797 - protein, 11 aa, synthetic construct
GNNMETLVPRQ

SEQ ID NO: 798 - protein, 11 aa, synthetic construct
GNGTLVHEPRQ

SEQ ID NO: 799 - protein, 11 aa, synthetic construct
GNSATPLLPRQ

SEQ ID NO: 800 - protein, 11 aa, synthetic construct
GNPASFIQYRQ

SEQ ID NO: 801 - protein, 11 aa, synthetic construct
GYVLGHMLARQ

SEQ ID NO: 802 - protein, 11 aa, synthetic construct
GNSDGHHSARQ

SEQ ID NO: 803 - protein, 11 aa, synthetic construct
GNTETYINSRQ

SEQ ID NO: 804 - protein, 11 aa, synthetic construct
GNMAETDYVRQ

SEQ ID NO: 805 - protein, 11 aa, synthetic construct
GNLNTVVLARQ

SEQ ID NO: 806 - protein, 11 aa, synthetic construct
GNGLSSTLERQ

SEQ ID NO: 807 - protein, 11 aa, synthetic construct
GNTNTTHGDRQ

SEQ ID NO: 808 - protein, 11 aa, synthetic construct
GNSPCRLIRRQ

SEQ ID NO: 809 - protein, 11 aa, synthetic construct
GNNLQSLPSRQ

SEQ ID NO: 810 - protein, 11 aa, synthetic construct
GNLPQTLLARQ

SEQ ID NO: 811 - protein, 11 aa, synthetic construct
GNTLGSLPLRQ

SEQ ID NO: 812 - protein, 11 aa, synthetic construct
GNLVESVHSRQ

SEQ ID NO: 813 - protein, 11 aa, synthetic construct
GNLLRTALTRQ

SEQ ID NO: 814 - protein, 11 aa, synthetic construct
GNSPPYNVSRQ

SEQ ID NO: 815 - protein, 11 aa, synthetic construct
GNLSVSYQQRQ

SEQ ID NO: 816 - protein, 11 aa, synthetic construct
GNTFGQSLDRQ

SEQ ID NO: 817 - protein, 11 aa, synthetic construct
GNQNVLTQNRQ

SEQ ID NO: 818 - protein, 11 aa, synthetic construct
GNTLSHSRTRQ

SEQ ID NO: 819 - protein, 11 aa, synthetic construct
GNLELSHQGRQ

SEQ ID NO: 820 - protein, 11 aa, synthetic construct
GNGDYEHNPRQ

SEQ ID NO: 821 - protein, 11 aa, synthetic construct
GNLHFSGVYRQ

SEQ ID NO: 822 - protein, 11 aa, synthetic construct
GNAIRGEAPRQ

SEQ ID NO: 823 - protein, 11 aa, synthetic construct
GNNSVHLPQRQ

SEQ ID NO: 824 - protein, 11 aa, synthetic construct
GNDGSSYPIRQ

SEQ ID NO: 825 - protein, 11 aa, synthetic construct
GNAQAQLSERQ

SEQ ID NO: 826 - protein, 11 aa, synthetic construct
GNLTDQFPQRQ

SEQ ID NO: 827 - protein, 11 aa, synthetic construct
GNRHTTQPSRQ

SEQ ID NO: 828 - protein, 11 aa, synthetic construct
GNSASYMADRQ

SEQ ID NO: 829 - protein, 11 aa, synthetic construct
GNSNRNEPPRQ

SEQ ID NO: 830 - protein, 11 aa, synthetic construct
GNLLVLGSGRQ

SEQ ID NO: 831 - protein, 11 aa, synthetic construct
GNLGHMGTERQ

SEQ ID NO: 832 - protein, 11 aa, synthetic construct
GNMALSMSERQ

SEQ ID NO: 833 - protein, 11 aa, synthetic construct
GNFMAHMTSRQ

SEQ ID NO: 834 - protein, 11 aa, synthetic construct
GNFVSMTTERQ

SEQ ID NO: 835 - protein, 11 aa, synthetic construct
GNGQRGLLGRQ

SEQ ID NO: 836 - protein, 11 aa, synthetic construct
GNVHETDPCRQ

SEQ ID NO: 837 - protein, 11 aa, synthetic construct
GNDSLGSSSRQ

SEQ ID NO: 838 - protein, 11 aa, synthetic construct
GNPGRMNFSRQ

SEQ ID NO: 839 - protein, 11 aa, synthetic construct
GNFGPQTQARQ

SEQ ID NO: 840 - protein, 11 aa, synthetic construct
GNNALSEHYRQ

SEQ ID NO: 841 - protein, 11 aa, synthetic construct
GNMPKSDAPRQ

SEQ ID NO: 842 - protein, 11 aa, synthetic construct
GNPALYIVLRQ

SEQ ID NO: 843 - protein, 11 aa, synthetic construct
GNYTSNLTARQ

SEQ ID NO: 844 - protein, 11 aa, synthetic construct
GNTVKPISTRQ

SEQ ID NO: 845 - protein, 11 aa, synthetic construct
GNNSAQFPIRQ

SEQ ID NO: 846 - protein, 11 aa, synthetic construct
GNNLRTGLTRQ

SEQ ID NO: 847 - protein, 11 aa, synthetic construct
GNMPTISHSRQ

SEQ ID NO: 848 - protein, 11 aa, synthetic construct
GNYTSNLMMRQ

SEQ ID NO: 849 - protein, 11 aa, synthetic construct
GNSHSSEFSRQ

SEQ ID NO: 850 - protein, 11 aa, synthetic construct
GNLTKMSPTRQ

SEQ ID NO: 851 - protein, 11 aa, synthetic construct
GNEHQSLLSRQ

SEQ ID NO: 852 - protein, 11 aa, synthetic construct
GNQPCGFPMRQ

SEQ ID NO: 853 - protein, 11 aa, synthetic construct
GNTADYSVPRQ

SEQ ID NO: 854 - protein, 11 aa, synthetic construct
GNTKSIPSTRQ

SEQ ID NO: 855 - protein, 11 aa, synthetic construct
GNTPTSQDTRQ

SEQ ID NO: 856 - protein, 11 aa, synthetic construct
GNLPYLLYPRQ

SEQ ID NO: 857 - protein, 11 aa, synthetic construct
GNSVGHTFVRQ

SEQ ID NO: 858 - protein, 11 aa, synthetic construct
GNLGDKKVKRQ

SEQ ID NO: 859 - protein, 11 aa, synthetic construct
GNSGPLISERQ

SEQ ID NO: 860 - protein, 11 aa, synthetic construct
GNTTSNEITRQ

SEQ ID NO: 861 - protein, 11 aa, synthetic construct
GNKPISTLSRQ

SEQ ID NO: 862 - protein, 11 aa, synthetic construct
GNGLERSESRQ

SEQ ID NO: 863 - protein, 11 aa, synthetic construct
GNAVMQYVPRQ

SEQ ID NO: 864 - protein, 11 aa, synthetic construct
GNGMPGTHQRQ

SEQ ID NO: 865 - protein, 11 aa, synthetic construct
GNLPHTQPLRQ

SEQ ID NO: 866 - protein, 11 aa, synthetic construct
GNSTPRLDSRQ

SEQ ID NO: 867 - protein, 11 aa, synthetic construct
CNMNTQLSPRQ

SEQ ID NO: 868 - protein, 11 aa, synthetic construct
GNYTSVQLNRQ

SEQ ID NO: 869 - protein, 11 aa, synthetic construct
GNNPREGKSRQ

SEQ ID NO: 870 - protein, 11 aa, synthetic construct
GNVMPQVLSRQ

SEQ ID NO: 871 - protein, 11 aa, synthetic construct
GNVLIVGAQRQ

SEQ ID NO: 872 - protein, 11 aa, synthetic construct
GNTNHTFTSRQ

SEQ ID NO: 873 - protein, 11 aa, synthetic construct
GNAHIASTVRQ

SEQ ID NO: 874 - protein, 11 aa, synthetic construct
GNTMPPTQPRQ

SEQ ID NO: 875 - protein, 11 aa, synthetic construct
GNPPVGINMRQ

SEQ ID NO: 876 - protein, 11 aa, synthetic construct
GNNMTASPLRQ

SEQ ID NO: 877 - protein, 11 aa, synthetic construct
GNSVKTAVMRQ

SEQ ID NO: 878 - protein, 11 aa, synthetic construct
GNPTEHVPPRQ

SEQ ID NO: 879 - protein, 11 aa, synthetic construct
GNSTKSTSSRQ

SEQ ID NO: 880 - protein, 11 aa, synthetic construct
GNAKIQRVARQ

SEQ ID NO: 881 - protein, 11 aa, synthetic construct
GNNIVIVVTRQ

SEQ ID NO: 882 - protein, 11 aa, synthetic construct
GNLLAPNLSRQ

SEQ ID NO: 883 - protein, 11 aa, synthetic construct
GNDTLSLGSRQ

SEQ ID NO: 884 - protein, 11 aa, synthetic construct
GNGNTTSLSRQ

SEQ ID NO: 885 - protein, 11 aa, synthetic construct
CNMPNTILSRQ

SEQ ID NO: 886 - protein, 11 aa, synthetic construct
GNGIQLTSSRQ

SEQ ID NO: 887 - protein, 11 aa, synthetic construct
GNTPQSSMFRQ

SEQ ID NO: 888 - protein, 11 aa, synthetic construct
CNLTSGLAGRQ

SEQ ID NO: 889 - protein, 11 aa, synthetic construct
GNLDRTTLPRQ

SEQ ID NO: 890 - protein, 11 aa, synthetic construct
GNVLREQPPRQ

SEQ ID NO: 891 - protein, 11 aa, synthetic construct
GNHSLMHHSRQ

SEQ ID NO: 892 - protein, 11 aa, synthetic construct
GNWEGHSIFRQ

SEQ ID NO: 893 - protein, 11 aa, synthetic construct
GNTGQIHPERQ

SEQ ID NO: 894 - protein, 11 aa, synthetic construct
GNHLSSVISRQ

SEQ ID NO: 895 - protein, 11 aa, synthetic construct
GNSMVVSTPRQ

SEQ ID NO: 896 - protein, 11 aa, synthetic construct
GNYPTSQPPRQ

SEQ ID NO: 897 - protein, 11 aa, synthetic construct
GNLTTIPPPRQ

SEQ ID NO: 898 - protein, 11 aa, synthetic construct
GNIKLYSQSRQ

SEQ ID NO: 899 - protein, 11 aa, synthetic construct
GNTPPLNMQRQ

SEQ ID NO: 900 - protein, 11 aa, synthetic construct
GNCTPPSTHRQ

SEQ ID NO: 901 - protein, 11 aa, synthetic construct
GNLHRFQEPRQ

SEQ ID NO: 902 - protein, 11 aa, synthetic construct
GNFGYSMESRQ

SEQ ID NO: 903 - protein, 11 aa, synthetic construct
GNTLQTLAERQ

SEQ ID NO: 904 - protein, 11 aa, synthetic construct
GNILGQSSIRQ

SEQ ID NO: 905 - protein, 11 aa, synthetic construct
GNLSASMVSRQ

SEQ ID NO: 906 - protein, 11 aa, synthetic construct
GNSESMMHGRQ

SEQ ID NO: 907 - protein, 11 aa, synthetic construct
GNEILTPVIRQ

SEQ ID NO: 908 - protein, 11 aa, synthetic construct
GNSLQPSPLRL

SEQ ID NO: 909 - protein, 11 aa, synthetic construct
GNLESYSGWRQ

SEQ ID NO: 910 - protein, 11 aa, synthetic construct
GNHDRNYTPRQ

SEQ ID NO: 911 - protein, 11 aa, synthetic construct
GNEGTAYSARQ

SEQ ID NO: 912 - protein, 11 aa, synthetic construct
GNEAQPHTSRQ

SEQ ID NO: 913 - protein, 11 aa, synthetic construct
GNSERVPTRRQ

SEQ ID NO: 914 - protein, 11 aa, synthetic construct
GNTANIGGPRQ

SEQ ID NO: 915 - protein, 11 aa, synthetic construct
GNHHCLPGSRQ

SEQ ID NO: 916 - protein, 11 aa, synthetic construct
GNHLSSTEPRQ

SEQ ID NO: 917 - protein, 11 aa, synthetic construct
GNDQPISAPRQ

SEQ ID NO: 918 - protein, 11 aa, synthetic construct
GNTQKFHGLRQ

SEQ ID NO: 919 - protein, 11 aa, synthetic construct
GNWSTYMNSRQ

SEQ ID NO: 920 - protein, 11 aa, synthetic construct
GNMVNVDHARQ

SEQ ID NO: 921 - protein, 11 aa, synthetic construct
GNAPPTSTPRQ

SEQ ID NO: 922 - protein, 11 aa, synthetic construct
GNTHGQTDHRQ

SEQ ID NO: 923 - protein, 11 aa, synthetic construct
GNIKIPSQGRQ

SEQ ID NO: 924 - protein, 11 aa, synthetic construct
GNGGSQQLERQ

SEQ ID NO: 925 - protein, 11 aa, synthetic construct
GNEISNSFRRQ

SEQ ID NO: 926 - protein, 11 aa, synthetic construct
GNVAYQMSPRQ

SEQ ID NO: 927 - protein, 11 aa, synthetic construct
GNSRNASYDRQ

SEQ ID NO: 928 - protein, 11 aa, synthetic construct
GNATVGTQPRQ

SEQ ID NO: 929 - protein, 11 aa, synthetic construct
GNSTTLHYPRQ

SEQ ID NO: 930 - protein, 11 aa, synthetic construct
GNGTPPNFSRQ

SEQ ID NO: 931 - protein, 11 aa, synthetic construct
GNTQGSANVRQ

SEQ ID NO: 932 - protein, 11 aa, synthetic construct
GNITIASLNRQ

SEQ ID NO: 933 - protein, 11 aa, synthetic construct
GNYSQDATARQ

SEQ ID NO: 934 - protein, 11 aa, synthetic construct
GNKPVSAQARQ

SEQ ID NO: 935 - protein, 11 aa, synthetic construct
GNIFAGLTSRQ

SEQ ID NO: 936 - protein, 11 aa, synthetic construct
GNLTAMLLSRQ

SEQ ID NO: 937 - protein, 11 aa, synthetic construct
GNKMGMRLSRQ

SEQ ID NO: 938 - protein, 11 aa, synthetic construct
GNTVRANQKRQ

SEQ ID NO: 939 - protein, 11 aa, synthetic construct
GNHAAGREERQ

SEQ ID NO: 940 - protein, 11 aa, synthetic construct
GNISLEIFRRQ

SEQ ID NO: 941 - protein, 11 aa, synthetic construct
GNRGSQGLSRQ

SEQ ID NO: 942 - protein, 11 aa, synthetic construct
GNLAEYPGQRQ

SEQ ID NO: 943 - protein, 11 aa, synthetic construct
GNSQLGTVTRQ

SEQ ID NO: 944 - protein, 11 aa, synthetic construct
GNSMSASEPRQ

SEQ ID NO: 945 - protein, 11 aa, synthetic construct
GNTQAFSSDRQ

SEQ ID NO: 946 - protein, 11 aa, synthetic construct
GNKNNPQMTRQ

SEQ ID NO: 947 - protein, 11 aa, synthetic construct
GNSQTDTAGRQ

SEQ ID NO: 948 - protein, 11 aa, synthetic construct
GNLFPNASPRQ

SEQ ID NO: 949 - protein, 11 aa, synthetic construct
GNTHTLSVERQ

SEQ ID NO: 950 - protein, 11 aa, synthetic construct
GNGGGFGLERQ

SEQ ID NO: 951 - protein, 11 aa, synthetic construct
GNLSRTIQPRQ

SEQ ID NO: 952 - protein, 11 aa, synthetic construct
GNNAASLLPRQ

SEQ ID NO: 953 - protein, 11 aa, synthetic construct
GNSSIGMPVRQ

SEQ ID NO: 954 - protein, 11 aa, synthetic construct
GNLGIASHERQ

SEQ ID NO: 955 - protein, 11 aa, synthetic construct
GNETSPHINRQ

SEQ ID NO: 956 - protein, 11 aa, synthetic construct
GNVALQGGPRQ

SEQ ID NO: 957 - protein, 11 aa, synthetic construct
GNVPISNLARQ

SEQ ID NO: 958 - protein, 11 aa, synthetic construct
GNFSRTMQDRQ

SEQ ID NO: 959 - protein, 11 aa, synthetic construct
GNSLQTQPQRR

SEQ ID NO: 960 - protein, 11 aa, synthetic construct
CNLAPAILSRQ

SEQ ID NO: 961 - protein, 11 aa, synthetic construct
GNLDMSSSLRQ

SEQ ID NO: 962 - protein, 11 aa, synthetic construct
GNQTNQSLTRQ

SEQ ID NO: 963 - protein, 11 aa, synthetic construct
GNFSSTGVPRQ

SEQ ID NO: 964 - protein, 11 aa, synthetic construct
GNSVQSLASRQ

SEQ ID NO: 965 - protein, 11 aa, synthetic construct
GNEINRSTPRQ

SEQ ID NO: 966 - protein, 11 aa, synthetic construct
GNAPTHQMSRQ

SEQ ID NO: 967 - protein, 11 aa, synthetic construct
GNNALLQCARQ

SEQ ID NO: 968 - protein, 11 aa, synthetic construct
GNSKISIAGRQ

SEQ ID NO: 969 - protein, 11 aa, synthetic construct
GNVDSHTTIRQ

SEQ ID NO: 970 - protein, 11 aa, synthetic construct
GNKGPLTDMRQ

SEQ ID NO: 971 - protein, 11 aa, synthetic construct
GNTTHQSPTRQ

SEQ ID NO: 972 - protein, 11 aa, synthetic construct
GNIDSSMAMRQ

SEQ ID NO: 973 - protein, 11 aa, synthetic construct
GNLGKQPTQRQ

SEQ ID NO: 974 - protein, 11 aa, synthetic construct
GNSRGPNTLRQ

SEQ ID NO: 975 - protein, 11 aa, synthetic construct
GNAILSSPARQ

SEQ ID NO: 976 - protein, 7 aa, synthetic construct
CLCTFLT

SEQ ID NO: 977 - protein, 7 aa, synthetic construct
FQLESMN

SEQ ID NO: 978 - protein, 7 aa, synthetic construct
PVAGLPP

SEQ ID NO: 979 - protein, 7 aa, synthetic construct
VVSGLAA

SEQ ID NO: 980 - protein, 7 aa, synthetic construct
LAGKPPP

SEQ ID NO: 981 - protein, 7 aa, synthetic construct
IMGPHMT

SEQ ID NO: 982 - protein, 7 aa, synthetic construct
LLGTALT

SEQ ID NO: 983 - protein, 7 aa, synthetic construct
YQLERMN

SEQ ID NO: 984 - protein, 7 aa, synthetic construct
LLGTSMQ

SEQ ID NO: 985 - protein, 7 aa, synthetic construct
LLGKSME

SEQ ID NO: 986 - protein, 7 aa, synthetic construct
ILGSSLE

SEQ ID NO: 987 - protein, 7 aa, synthetic construct
ILSPSLA

SEQ ID NO: 988 - protein, 7 aa, synthetic construct
LLGANMV

SEQ ID NO: 989 - protein, 7 aa, synthetic construct
IVAGLKS

SEQ ID NO: 990 - protein, 7 aa, synthetic construct
LLGPYMQ

SEQ ID NO: 991 - protein, 7 aa, synthetic construct
VLGYNLQ

SEQ ID NO: 992 - protein, 7 aa, synthetic construct
LLGKTME

SEQ ID NO: 993 - protein, 7 aa, synthetic construct
LLGNALT

SEQ ID NO: 994 - protein, 7 aa, synthetic construct
ILGITLA

SEQ ID NO: 995 - protein, 7 aa, synthetic construct
ILGSTLA

SEQ ID NO: 996 - protein, 7 aa, synthetic construct
QVPGLGV

SEQ ID NO: 997 - protein, 7 aa, synthetic construct
HNVSIPA

SEQ ID NO: 998 - protein, 7 aa, synthetic construct
PSRECGL

SEQ ID NO: 999 - protein, 7 aa, synthetic construct
LLGPNLS

SEQ ID NO: 1000 - protein, 7 aa, synthetic construct
LLGTAMT

SEQ ID NO: 1001 - protein, 7 aa, synthetic construct
VVSGIAA

SEQ ID NO: 1002 - protein, 7 aa, synthetic construct
IMGPHLA

SEQ ID NO: 1003 - protein, 7 aa, synthetic construct
LLGPSMQ

SEQ ID NO: 1004 - protein, 7 aa, synthetic construct
VVSGLAA

SEQ ID NO: 1005 - protein, 7 aa, synthetic construct
IMGPHLT

SEQ ID NO: 1006 - protein, 7 aa, synthetic construct
LFGPSMA

SEQ ID NO: 1007 - protein, 7 aa, synthetic construct
VVSGLSA

SEQ ID NO: 1008 - protein, 7 aa, synthetic construct
QVPGLGV

SEQ ID NO: 1009 - protein, 7 aa, synthetic construct
ILGSGLT

SEQ ID NO: 1010 - protein, 7 aa, synthetic construct
IMGPLLT

SEQ ID NO: 1011 - protein, 7 aa, synthetic construct
LLGTAME

SEQ ID NO: 1012 - protein, 7 aa, synthetic construct
GLGPALV

SEQ ID NO: 1013 - protein, 7 aa, synthetic construct
ILGSSLE

SEQ ID NO: 1014 - protein, 7 aa, synthetic construct
IVAGLAQ

SEQ ID NO: 1015 - protein, 7 aa, synthetic construct
PVAGLTP

SEQ ID NO: 1016 - protein, 7 aa, synthetic construct
IFGRDMA

SEQ ID NO: 1017 - protein, 7 aa, synthetic construct
ILGPSNA

SEQ ID NO: 1018 - protein, 7 aa, synthetic construct
LLGPTMQ

SEQ ID NO: 1019 - protein, 7 aa, synthetic construct
MSQKIPK

SEQ ID NO: 1020 - protein, 7 aa, synthetic construct
IVSGLAG

SEQ ID NO: 1021 - protein, 7 aa, synthetic construct
TVPGLGH

SEQ ID NO: 1022 - protein, 7 aa, synthetic construct
LLGTSLT

SEQ ID NO: 1023 - protein, 7 aa, synthetic construct
LLGHSMQ

SEQ ID NO: 1024 - protein, 7 aa, synthetic construct
LLGSNLE

SEQ ID NO: 1025 - protein, 7 aa, synthetic construct
PVPGLSS

SEQ ID NO: 1026 - protein, 7 aa, synthetic construct
FQLERMN

SEQ ID NO: 1027 - protein, 7 aa, synthetic construct
VLGSALS

SEQ ID NO: 1028 - protein, 7 aa, synthetic construct
VLGHILA

SEQ ID NO: 1029 - protein, 7 aa, synthetic construct
VVPGLSY

SEQ ID NO: 1030 - protein, 7 aa, synthetic construct
ELDLSMA

SEQ ID NO: 1031 - protein, 7 aa, synthetic construct
FQLDRMN

SEQ ID NO: 1032 - protein, 7 aa, synthetic construct
LLGPSMQ

SEQ ID NO: 1033 - protein, 7 aa, synthetic construct
VVPGLSF

SEQ ID NO: 1034 - protein, 7 aa, synthetic construct
VLGQQMV

SEQ ID NO: 1035 - protein, 7 aa, synthetic construct
LLGTTLS

SEQ ID NO: 1036 - protein, 7 aa, synthetic construct
ILGSSLS

SEQ ID NO: 1037 - protein, 7 aa, synthetic construct
IVSGLAG

SEQ ID NO: 1038 - protein, 7 aa, synthetic construct
ILGSSLT

SEQ ID NO: 1039 - protein, 7 aa, synthetic construct
LLGTPMS

SEQ ID NO: 1040 - protein, 7 aa, synthetic construct
LLGSALT

SEQ ID NO: 1041 - protein, 7 aa, synthetic construct
IMGPHLN

SEQ ID NO: 1042 - protein, 7 aa, synthetic construct
ILGHSLE

SEQ ID NO: 1043 - protein, 7 aa, synthetic construct
VLGHMMA

SEQ ID NO: 1044 - protein, 7 aa, synthetic construct
IMGPHLT

SEQ ID NO: 1045 - protein, 7 aa, synthetic construct
ILGVVLS

SEQ ID NO: 1046 - protein, 7 aa, synthetic construct
LLGVQMQ

SEQ ID NO: 1047 - protein, 7 aa, synthetic construct
VLGPQLT

SEQ ID NO: 1048 - protein, 7 aa, synthetic construct
IMGPHLS

SEQ ID NO: 1049 - protein, 7 aa, synthetic construct
LLGSTMS

SEQ ID NO: 1050 - protein, 7 aa, synthetic construct
LLGKALV

SEQ ID NO: 1051 - protein, 7 aa, synthetic construct
VHGAALI

SEQ ID NO: 1052 - protein, 7 aa, synthetic construct
LLGPHLS

SEQ ID NO: 1053 - protein, 7 aa, synthetic construct
PIPGLPT

SEQ ID NO: 1054 - protein, 7 aa, synthetic construct
HTRSVDI

SEQ ID NO: 1055 - protein, 7 aa, synthetic construct
LLGPSMR

SEQ ID NO: 1056 - protein, 7 aa, synthetic construct
NIANVQV

SEQ ID NO: 1057 - protein, 7 aa, synthetic construct
VLGQMLA

SEQ ID NO: 1058 - protein, 7 aa, synthetic construct
VVTGLAA

SEQ ID NO: 1059 - protein, 7 aa, synthetic construct
LLGPSLQ

SEQ ID NO: 1060 - protein, 7 aa, synthetic construct
IMGPHLT

SEQ ID NO: 1061 - protein, 7 aa, synthetic construct
NHTREDL

SEQ ID NO: 1062 - protein, 7 aa, synthetic construct
LSVGLDA

SEQ ID NO: 1063 - protein, 7 aa, synthetic construct
LGLTFSH

SEQ ID NO: 1064 - protein, 7 aa, synthetic construct
IMGATLT

SEQ ID NO: 1065 - protein, 7 aa, synthetic construct
VLGHMLA

SEQ ID NO: 1066 - protein, 7 aa, synthetic construct
VMGPALT

SEQ ID NO: 1067 - protein, 7 aa, synthetic construct
LLGPSMQ

SEQ ID NO: 1068 - protein, 7 aa, synthetic construct
VLGHSLS

SEQ ID NO: 1069 - protein, 7 aa, synthetic construct
SVAGLGP

SEQ ID NO: 1070 - protein, 7 aa, synthetic construct
HVPGLGV

SEQ ID NO: 1071 - protein, 7 aa, synthetic construct
VLGHMLA

SEQ ID NO: 1072 - protein, 7 aa, synthetic construct
IVSGLSG

SEQ ID NO: 1073 - protein, 7 aa, synthetic construct
IVAGLSS

SEQ ID NO: 1074 - protein, 7 aa, synthetic construct
LVPGLSA

SEQ ID NO: 1075 - protein, 7 aa, synthetic construct
FQLERLN

SEQ ID NO: 1076 - protein, 7 aa, synthetic construct
VLGHMLA

SEQ ID NO: 1077 - protein, 7 aa, synthetic construct
WVPPQTP

SEQ ID NO: 1078 - protein, 7 aa, synthetic construct
VLGHLLA

SEQ ID NO: 1079 - protein, 7 aa, synthetic construct
VLGNMLA

SEQ ID NO: 1080 - protein, 7 aa, synthetic construct
ILGSSLT

SEQ ID NO: 1081 - protein, 7 aa, synthetic construct
VVSGLAS

SEQ ID NO: 1082 - protein, 7 aa, synthetic construct
VVPGLSY

SEQ ID NO: 1083 - protein, 7 aa, synthetic construct
VLGHMLE

SEQ ID NO: 1084 - protein, 7 aa, synthetic construct
VDSNQVS

SEQ ID NO: 1085 - protein, 7 aa, synthetic construct
IVSGLSA

SEQ ID NO: 1086 - protein, 7 aa, synthetic construct
LLGTALS

SEQ ID NO: 1087 - protein, 7 aa, synthetic construct
ILGTTLS

SEQ ID NO: 1088 - protein, 7 aa, synthetic construct
VFGSSLV

SEQ ID NO: 1089 - protein, 7 aa, synthetic construct
LLGSALN

SEQ ID NO: 1090 - protein, 7 aa, synthetic construct
LLGPTLS

SEQ ID NO: 1091 - protein, 7 aa, synthetic construct
ILGHGML

SEQ ID NO: 1092 - protein, 7 aa, synthetic construct
LLGPSMQ

SEQ ID NO: 1093 - protein, 7 aa, synthetic construct
NVAASPS

SEQ ID NO: 1094 - protein, 7 aa, synthetic construct
NFTADPL

SEQ ID NO: 1095 - protein, 7 aa, synthetic construct
GDSGISS

SEQ ID NO: 1096 - protein, 7 aa, synthetic construct
ILGSSLT

SEQ ID NO: 1097 - protein, 7 aa, synthetic construct
TNTTHLE

SEQ ID NO: 1098 - protein, 7 aa, synthetic construct
IMGAHLT

SEQ ID NO: 1099 - protein, 7 aa, synthetic construct
VLGVHLT

SEQ ID NO: 1100 - protein, 7 aa, synthetic construct
ILGSTLT

SEQ ID NO: 1101 - protein, 7 aa, synthetic construct
ILGELLS

SEQ ID NO: 1102 - protein, 7 aa, synthetic construct
ILGNIMS

SEQ ID NO: 1103 - protein, 7 aa, synthetic construct
VLGYNMQ

SEQ ID NO: 1104 - protein, 7 aa, synthetic construct
ILGNILS

SEQ ID NO: 1105 - protein, 7 aa, synthetic construct
ADPPQWK

SEQ ID NO: 1106 - protein, 7 aa, synthetic construct
LLGTQLA

SEQ ID NO: 1107 - protein, 7 aa, synthetic construct
VLGSAMK

SEQ ID NO: 1108 - protein, 7 aa, synthetic construct
LLGSSLS

SEQ ID NO: 1109 - protein, 7 aa, synthetic construct
FCSVNKE

SEQ ID NO: 1110 - protein, 7 aa, synthetic construct
LLGPNLC

SEQ ID NO: 1111 - protein, 7 aa, synthetic construct
FQLERMN

SEQ ID NO: 1112 - protein, 7 aa, synthetic construct
PVSGLPP

SEQ ID NO: 1113 - protein, 7 aa, synthetic construct
LLGPNLS

SEQ ID NO: 1114 - protein, 7 aa, synthetic construct
LLGRNLS

SEQ ID NO: 1115 - protein, 7 aa, synthetic construct
SIPPAAK

SEQ ID NO: 1116 - protein, 7 aa, synthetic construct
VLGFNMQ

SEQ ID NO: 1117 - protein, 7 aa, synthetic construct
PIPGLGA

SEQ ID NO: 1118 - protein, 7 aa, synthetic construct
LLGTNLS

SEQ ID NO: 1119 - protein, 7 aa, synthetic construct
LLGPNLS

SEQ ID NO: 1120 - protein, 7 aa, synthetic construct
APPLQFS

SEQ ID NO: 1121 - protein, 7 aa, synthetic construct
VMGSALA

SEQ ID NO: 1122 - protein, 7 aa, synthetic construct
LLGHNLS

SEQ ID NO: 1123 - protein, 7 aa, synthetic construct
LLGSSLT

SEQ ID NO: 1124 - protein, 7 aa, synthetic construct
VFGPGLI

SEQ ID NO: 1125 - protein, 7 aa, synthetic construct
IMGPTLT

SEQ ID NO: 1126 - protein, 7 aa, synthetic construct
VHGPALI

SEQ ID NO: 1127 - protein, 7 aa, synthetic construct
ILGSSLT

SEQ ID NO: 1128 - protein, 7 aa, synthetic construct
MLGPSMQ

SEQ ID NO: 1129 - protein, 7 aa, synthetic construct
IMGPHLT

SEQ ID NO: 1130 - protein, 7 aa, synthetic construct
QNPRIEQ

SEQ ID NO: 1131 - protein, 7 aa, synthetic construct
LLGPHLS

SEQ ID NO: 1132 - protein, 7 aa, synthetic construct
LLGHALT

SEQ ID NO: 1133 - protein, 7 aa, synthetic construct
IMGVSLN

SEQ ID NO: 1134 - protein, 7 aa, synthetic construct
IMGTHLT

SEQ ID NO: 1135 - protein, 7 aa, synthetic construct
LVPGIPA

SEQ ID NO: 1136 - protein, 7 aa, synthetic construct
QILAPLS

SEQ ID NO: 1137 - protein, 7 aa, synthetic construct
NVTTFPP

SEQ ID NO: 1138 - protein, 7 aa, synthetic construct
LLGPMLS

SEQ ID NO: 1139 - protein, 7 aa, synthetic construct
LLGQTLS

SEQ ID NO: 1140 - protein, 7 aa, synthetic construct
HASQFTF

SEQ ID NO: 1141 - protein, 7 aa, synthetic construct
TVPGLGH

SEQ ID NO: 1142 - protein, 7 aa, synthetic construct
SLPPPTK

SEQ ID NO: 1143 - protein, 7 aa, synthetic construct
NVSQQPV

SEQ ID NO: 1144 - protein, 7 aa, synthetic construct
NIAHMQL

SEQ ID NO: 1145 - protein, 7 aa, synthetic construct
ILGEVLT

SEQ ID NO: 1146 - protein, 7 aa, synthetic construct
ILGPHLD

SEQ ID NO: 1147 - protein, 7 aa, synthetic construct
IMGPHLT

SEQ ID NO: 1148 - protein, 7 aa, synthetic construct
VLGSALN

SEQ ID NO: 1149 - protein, 7 aa, synthetic construct
ILGSSLT

SEQ ID NO: 1150 - protein, 7 aa, synthetic construct
LLGHMLA

SEQ ID NO: 1151 - protein, 7 aa, synthetic construct
ILGTSLT

SEQ ID NO: 1152 - protein, 7 aa, synthetic construct
ILGSSLV

SEQ ID NO: 1153 - protein, 7 aa, synthetic construct
LLGPNLT

SEQ ID NO: 1154 - protein, 7 aa, synthetic construct
SDNATHS

SEQ ID NO: 1155 - protein, 7 aa, synthetic construct
VSPPTFT

SEQ ID NO: 1156 - protein, 7 aa, synthetic construct
NITGTPV

SEQ ID NO: 1157 - protein, 7 aa, synthetic construct
LLGPTLS

SEQ ID NO: 1158 - protein, 7 aa, synthetic construct
SVPGLGH

SEQ ID NO: 1159 - protein, 7 aa, synthetic construct
IVAGLAQ

SEQ ID NO: 1160 - protein, 7 aa, synthetic construct
MLGPNLS

SEQ ID NO: 1161 - protein, 7 aa, synthetic construct
ILGSSLT

SEQ ID NO: 1162 - protein, 7 aa, synthetic construct
VVSGLAD

SEQ ID NO: 1163 - protein, 7 aa, synthetic construct
DNRLILD

SEQ ID NO: 1164 - protein, 7 aa, synthetic construct
ILGPHLT

SEQ ID NO: 1165 - protein, 7 aa, synthetic construct
NLSHDPL

SEQ ID NO: 1166 - protein, 7 aa, synthetic construct
LLGPGLT

SEQ ID NO: 1167 - protein, 7 aa, synthetic construct
EDSNRTM

SEQ ID NO: 1168 - protein, 7 aa, synthetic construct
NLSASPL

SEQ ID NO: 1169 - protein, 7 aa, synthetic construct
GECHLSF

SEQ ID NO: 1170 - protein, 7 aa, synthetic construct
QNPKIPS

SEQ ID NO: 1171 - protein, 7 aa, synthetic construct
VVPGLSN

SEQ ID NO: 1172 - protein, 7 aa, synthetic construct
IIPGLVA

SEQ ID NO: 1173 - protein, 7 aa, synthetic construct
MQLSSLN

SEQ ID NO: 1174 - protein, 7 aa, synthetic construct
LLGTALT

SEQ ID NO: 1175 - protein, 7 aa, synthetic construct
VLGHMLS

SEQ ID NO: 1176 - protein, 7 aa, synthetic construct
ILGEVLS

SEQ ID NO: 1177 - protein, 7 aa, synthetic construct
LPAGIHG

SEQ ID NO: 1178 - protein, 7 aa, synthetic construct
IMGHHLT

SEQ ID NO: 1179 - protein, 7 aa, synthetic construct
LLGPSMK

SEQ ID NO: 1180 - protein, 7 aa, synthetic construct
LGTPFTP

SEQ ID NO: 1181 - protein, 7 aa, synthetic construct
PVPGLTT

SEQ ID NO: 1182 - protein, 7 aa, synthetic construct
IVSGLAQ

SEQ ID NO: 1183 - protein, 7 aa, synthetic construct
VLGHKLA

SEQ ID NO: 1184 - protein, 7 aa, synthetic construct
NVTRAEL

SEQ ID NO: 1185 - protein, 7 aa, synthetic construct
FLGPSMQ

SEQ ID NO: 1186 - protein, 7 aa, synthetic construct
NISQDPP

SEQ ID NO: 1187 - protein, 7 aa, synthetic construct
TVPGLGL

SEQ ID NO: 1188 - protein, 7 aa, synthetic construct
FLGPNLS

SEQ ID NO: 1189 - protein, 7 aa, synthetic construct
IIPGLVA

SEQ ID NO: 1190 - protein, 7 aa, synthetic construct
NTVRAEL

SEQ ID NO: 1191 - protein, 7 aa, synthetic construct
MASTLGP

SEQ ID NO: 1192 - protein, 7 aa, synthetic construct
LLGPSMQ

SEQ ID NO: 1193 - protein, 7 aa, synthetic construct
SRSFGNP

SEQ ID NO: 1194 - protein, 7 aa, synthetic construct
VDRNSTS

SEQ ID NO: 1195 - protein, 7 aa, synthetic construct
IVAGLSQ

SEQ ID NO: 1196 - protein, 7 aa, synthetic construct
ILGTSLV

SEQ ID NO: 1197 - protein, 7 aa, synthetic construct
LLGPEMT

SEQ ID NO: 1198 - protein, 7 aa, synthetic construct
LLGPVMA

SEQ ID NO: 1199 - protein, 7 aa, synthetic construct
VIPGLVV

SEQ ID NO: 1200 - protein, 7 aa, synthetic construct
IVAGLTS

SEQ ID NO: 1201 - protein, 7 aa, synthetic construct
LTGNQMR

SEQ ID NO: 1202 - protein, 7 aa, synthetic construct
NMSASPL

SEQ ID NO: 1203 - protein, 7 aa, synthetic construct
LSTTQLS

SEQ ID NO: 1204 - protein, 7 aa, synthetic construct
SVKDWTT

SEQ ID NO: 1205 - protein, 7 aa, synthetic construct
ILGEVMS

SEQ ID NO: 1206 - protein, 7 aa, synthetic construct
LFGNSMV

SEQ ID NO: 1207 - protein, 7 aa, synthetic construct
PVSGLAS

SEQ ID NO: 1208 - protein, 7 aa, synthetic construct
ILGTALT

SEQ ID NO: 1209 - protein, 7 aa, synthetic construct
LLGPNLI

SEQ ID NO: 1210 - protein, 7 aa, synthetic construct
LLGKSLT

SEQ ID NO: 1211 - protein, 7 aa, synthetic construct
SSNSVSL

SEQ ID NO: 1212 - protein, 7 aa, synthetic construct
ILGSSLM

SEQ ID NO: 1213 - protein, 7 aa, synthetic construct
VALSMHA

SEQ ID NO: 1214 - protein, 7 aa, synthetic construct
IMGPNLT

SEQ ID NO: 1215 - protein, 7 aa, synthetic construct
NIVAQQL

SEQ ID NO: 1216 - protein, 7 aa, synthetic construct
PRVSGSQ

SEQ ID NO: 1217 - protein, 7 aa, synthetic construct
SLDQSLP

SEQ ID NO: 1218 - protein, 7 aa, synthetic construct
ADSGTPE

SEQ ID NO: 1219 - protein, 7 aa, synthetic construct
ILGATLT

SEQ ID NO: 1220 - protein, 7 aa, synthetic construct
KNVSIAE

SEQ ID NO: 1221 - protein, 7 aa, synthetic construct
NEPHMGA

SEQ ID NO: 1222 - protein, 7 aa, synthetic construct
SAITSQD

SEQ ID NO: 1223 - protein, 7 aa, synthetic construct
SPNEAQF

SEQ ID NO: 1224 - protein, 7 aa, synthetic construct
NPFLASQ

SEQ ID NO: 1225 - protein, 7 aa, synthetic construct
NMSATPL

SEQ ID NO: 1226 - protein, 7 aa, synthetic construct
FYATSTS

SEQ ID NO: 1227 - protein, 7 aa, synthetic construct
ILGTHLT

SEQ ID NO: 1228 - protein, 7 aa, synthetic construct
QQAHEVG

SEQ ID NO: 1229 - protein, 7 aa, synthetic construct
PALYIFI

SEQ ID NO: 1230 - protein, 7 aa, synthetic construct
VLGLMLA

SEQ ID NO: 1231 - protein, 7 aa, synthetic construct
LLGPYLS

SEQ ID NO: 1232 - protein, 7 aa, synthetic construct
VLGYYMQ

SEQ ID NO: 1233 - protein, 7 aa, synthetic construct
LPPICHV

SEQ ID NO: 1234 - protein, 7 aa, synthetic construct
ISGNEIL

SEQ ID NO: 1235 - protein, 7 aa, synthetic construct
VLGHMLA

SEQ ID NO: 1236 - protein, 7 aa, synthetic construct
GGICPGA

SEQ ID NO: 1237 - protein, 7 aa, synthetic construct
VSIYTSS

SEQ ID NO: 1238 - protein, 7 aa, synthetic construct
LLGPTLT

SEQ ID NO: 1239 - protein, 7 aa, synthetic construct
PWMTSYV

SEQ ID NO: 1240 - protein, 7 aa, synthetic construct
GHRNNHL

SEQ ID NO: 1241 - protein, 7 aa, synthetic construct
THCSYPD

SEQ ID NO: 1242 - protein, 7 aa, synthetic construct
SLYMSSL

SEQ ID NO: 1243 - protein, 7 aa, synthetic construct
ALSCLWY

SEQ ID NO: 1244 - protein, 7 aa, synthetic construct
ILGNFMS

SEQ ID NO: 1245 - protein, 7 aa, synthetic construct
LVTRGSM

SEQ ID NO: 1246 - protein, 7 aa, synthetic construct
VLPGLNA

SEQ ID NO: 1247 - protein, 7 aa, synthetic construct
MLTCSVS

SEQ ID NO: 1248 - protein, 7 aa, synthetic construct
LSIRTHS

SEQ ID NO: 1249 - protein, 7 aa, synthetic construct
TLQNIYS

SEQ ID NO: 1250 - protein, 7 aa, synthetic construct
ITTPHKQ

SEQ ID NO: 1251 - protein, 7 aa, synthetic construct
VRPITFL

SEQ ID NO: 1252 - protein, 7 aa, synthetic construct
SDSFSLS

SEQ ID NO: 1253 - protein, 7 aa, synthetic construct
MPNTFLS

SEQ ID NO: 1254 - protein, 7 aa, synthetic construct
ILGEVLS

SEQ ID NO: 1255 - protein, 7 aa, synthetic construct
QSRVTRV

SEQ ID NO: 1256 - protein, 7 aa, synthetic construct
NVTSQRT

SEQ ID NO: 1257 - protein, 7 aa, synthetic construct
ILGYSLT

SEQ ID NO: 1258 - protein, 7 aa, synthetic construct
FGTNRTN

SEQ ID NO: 1259 - protein, 7 aa, synthetic construct
LNLLIGL

SEQ ID NO: 1260 - protein, 7 aa, synthetic construct
LVSKTKS

SEQ ID NO: 1261 - protein, 7 aa, synthetic construct
GGFTIYE

SEQ ID NO: 1262 - protein, 7 aa, synthetic construct
DPPLPYP

SEQ ID NO: 1263 - protein, 7 aa, synthetic construct
GMMVTVM

SEQ ID NO: 1264 - protein, 7 aa, synthetic construct
GSRYTPT

SEQ ID NO: 1265 - protein, 7 aa, synthetic construct
QSLLCSE

SEQ ID NO: 1266 - protein, 7 aa, synthetic construct
DSSYSLI

SEQ ID NO: 1267 - protein, 7 aa, synthetic construct
PHINKTL

SEQ ID NO: 1268 - protein, 7 aa, synthetic construct
LLGPSME

SEQ ID NO: 1269 - protein, 7 aa, synthetic construct
PAPHCSL

SEQ ID NO: 1270 - protein, 7 aa, synthetic construct
LLGPSMQ

SEQ ID NO: 1271 - protein, 7 aa, synthetic construct
LLGPLLS

SEQ ID NO: 1272 - protein, 7 aa, synthetic construct
QYQSLGP

SEQ ID NO: 1273 - protein, 7 aa, synthetic construct
LLGLALT

SEQ ID NO: 1274 - protein, 7 aa, synthetic construct
NTHTYMH

SEQ ID NO: 1275 - protein, 7 aa, synthetic construct
RLSTSPL

SEQ ID NO: 1276 - protein, 7 aa, synthetic construct
TPSSQDS

SEQ ID NO: 1277 - protein, 7 aa, synthetic construct
SISIKRS

SEQ ID NO: 1278 - protein, 7 aa, synthetic construct
PSNTESP

SEQ ID NO: 1279 - protein, 7 aa, synthetic construct
YQSTMYA

SEQ ID NO: 1280 - protein, 7 aa, synthetic construct
LNESYYS

SEQ ID NO: 1281 - protein, 7 aa, synthetic construct
MRLALPG

SEQ ID NO: 1282 - protein, 7 aa, synthetic construct
PSSRQQL

SEQ ID NO: 1283 - protein, 7 aa, synthetic construct
SAIFSVP

SEQ ID NO: 1284 - protein, 7 aa, synthetic construct
LLGPSML

SEQ ID NO: 1285 - protein, 7 aa, synthetic construct
SSSHEYS

SEQ ID NO: 1286 - protein, 7 aa, synthetic construct
LSTYQLS

SEQ ID NO: 1287 - protein, 7 aa, synthetic construct
AMNLLAY

SEQ ID NO: 1288 - protein, 7 aa, synthetic construct
QGTQQYE

SEQ ID NO: 1289 - protein, 7 aa, synthetic construct
GRSSSDE

SEQ ID NO: 1290 - protein, 7 aa, synthetic construct
TDQTATD

SEQ ID NO: 1291 - protein, 7 aa, synthetic construct
SLGPTLV

SEQ ID NO: 1292 - protein, 7 aa, synthetic construct
TIPGLQV

SEQ ID NO: 1293 - protein, 7 aa, synthetic construct
TRYLVPQ

SEQ ID NO: 1294 - protein, 7 aa, synthetic construct
YYSKLPL

SEQ ID NO: 1295 - protein, 7 aa, synthetic construct
YSVHLYP

SEQ ID NO: 1296 - protein, 7 aa, synthetic construct
YLAFTRF

SEQ ID NO: 1297 - protein, 7 aa, synthetic construct
WYLGEPV

SEQ ID NO: 1298 - protein, 7 aa, synthetic construct
WTISGIN

SEQ ID NO: 1299 - protein, 7 aa, synthetic construct
WHFHQSE

SEQ ID NO: 1300 - protein, 7 aa, synthetic construct
VVSCSYD

SEQ ID NO: 1301 - protein, 7 aa, synthetic construct
VSVECTR

SEQ ID NO: 1302 - protein, 7 aa, synthetic construct
VRTPGCN

SEQ ID NO: 1303 - protein, 7 aa, synthetic construct
VRSTAST

SEQ ID NO: 1304 - protein, 7 aa, synthetic construct
VQHTKRG

SEQ ID NO: 1305 - protein, 7 aa, synthetic construct
VMAGLCA

SEQ ID NO: 1306 - protein, 7 aa, synthetic construct
VLGTVLV

SEQ ID NO: 1307 - protein, 7 aa, synthetic construct
VLGRMLA

SEQ ID NO: 1308 - protein, 7 aa, synthetic construct
VFSMSTL

SEQ ID NO: 1309 - protein, 7 aa, synthetic construct
VCISQMN

SEQ ID NO: 1310 - protein, 7 aa, synthetic construct
VAYCPSQ

SEQ ID NO: 1311 - protein, 7 aa, synthetic construct
TPFRVVL

SEQ ID NO: 1312 - protein, 7 aa, synthetic construct
TLSHSWT

SEQ ID NO: 1313 - protein, 7 aa, synthetic construct
THSTFYP

SEQ ID NO: 1314 - protein, 7 aa, synthetic construct
TGRRDYQ

SEQ ID NO: 1315 - protein, 7 aa, synthetic construct
TFYSTHV

SEQ ID NO: 1316 - protein, 7 aa, synthetic construct
TDKRLST

SEQ ID NO: 1317 - protein, 7 aa, synthetic construct
TAPLLLL

SEQ ID NO: 1318 - protein, 7 aa, synthetic construct
STKRTDS

SEQ ID NO: 1319 - protein, 7 aa, synthetic construct
SRYAFMI

SEQ ID NO: 1320 - protein, 7 aa, synthetic construct
SRNASDD

SEQ ID NO: 1321 - protein, 7 aa, synthetic construct
SRLKHGD

SEQ ID NO: 1322 - protein, 7 aa, synthetic construct
SPTLSTF

SEQ ID NO: 1323 - protein, 7 aa, synthetic construct
SCIMSMR

SEQ ID NO: 1324 - protein, 7 aa, synthetic construct
SCANTVW

SEQ ID NO: 1325 - protein, 7 aa, synthetic construct
SASRVIL

SEQ ID NO: 1326 - protein, 7 aa, synthetic construct
RYNFSDS

SEQ ID NO: 1327 - protein, 7 aa, synthetic construct
RPGALSR

SEQ ID NO: 1328 - protein, 7 aa, synthetic construct
RNVLPQR

SEQ ID NO: 1329 - protein, 7 aa, synthetic construct
RISITIG

SEQ ID NO: 1330 - protein, 7 aa, synthetic construct
RFPTSPL

SEQ ID NO: 1331 - protein, 7 aa, synthetic construct
RALRMFL

SEQ ID NO: 1332 - protein, 7 aa, synthetic construct
QPWSVRR

SEQ ID NO: 1333 - protein, 7 aa, synthetic construct
QMTIIGS

SEQ ID NO: 1334 - protein, 7 aa, synthetic construct
PVYVRMV

SEQ ID NO: 1335 - protein, 7 aa, synthetic construct
PPTPEHS

SEQ ID NO: 1336 - protein, 7 aa, synthetic construct
PMLLVVS

SEQ ID NO: 1337 - protein, 7 aa, synthetic construct
PLLGSLM

SEQ ID NO: 1338 - protein, 7 aa, synthetic construct
PLCSSQG

SEQ ID NO: 1339 - protein, 7 aa, synthetic construct
PCCKYFI

SEQ ID NO: 1340 - protein, 7 aa, synthetic construct
PALPRNR

SEQ ID NO: 1341 - protein, 7 aa, synthetic construct
NFLGLIS

SEQ ID NO: 1342 - protein, 7 aa, synthetic construct
NCYPSDL

SEQ ID NO: 1343 - protein, 7 aa, synthetic construct
NCTIPSL

SEQ ID NO: 1344 - protein, 7 aa, synthetic construct
MSTSVWS

SEQ ID NO: 1345 - protein, 7 aa, synthetic construct
MSLTGTT

SEQ ID NO: 1346 - protein, 7 aa, synthetic construct
MRSPIGQ

SEQ ID NO: 1347 - protein, 7 aa, synthetic construct
MRIMVPW

SEQ ID NO: 1348 - protein, 7 aa, synthetic construct
MPLCTPT

SEQ ID NO: 1349 - protein, 7 aa, synthetic construct
MCKPALH

SEQ ID NO: 1350 - protein, 7 aa, synthetic construct
MACSLGP

SEQ ID NO: 1351 - protein, 7 aa, synthetic construct
LTYQFPP

SEQ ID NO: 1352 - protein, 7 aa, synthetic construct
LSPFLQK

SEQ ID NO: 1353 - protein, 7 aa, synthetic construct
LSLSMTI

SEQ ID NO: 1354 - protein, 7 aa, synthetic construct
LSLSIKP

SEQ ID NO: 1355 - protein, 7 aa, synthetic construct
LSLSFPS

SEQ ID NO: 1356 - protein, 7 aa, synthetic construct
LSGIGFS

SEQ ID NO: 1357 - protein, 7 aa, synthetic construct
LRYQKAM

SEQ ID NO: 1358 - protein, 7 aa, synthetic construct
LRFYYPY

SEQ ID NO: 1359 - protein, 7 aa, synthetic construct
LMTGWTS

SEQ ID NO: 1360 - protein, 7 aa, synthetic construct
LLSWQDM

SEQ ID NO: 1361 - protein, 7 aa, synthetic construct
LLDLSMA

SEQ ID NO: 1362 - protein, 7 aa, synthetic construct
LHSKGGA

SEQ ID NO: 1363 - protein, 7 aa, synthetic construct
LHRLMGH

SEQ ID NO: 1364 - protein, 7 aa, synthetic construct
LHPWQTP

SEQ ID NO: 1365 - protein, 7 aa, synthetic construct
LEFWSSF

SEQ ID NO: 1366 - protein, 7 aa, synthetic construct
LCYIYMT

SEQ ID NO: 1367 - protein, 7 aa, synthetic construct
KEIARVR

SEQ ID NO: 1368 - protein, 7 aa, synthetic construct
IYILKRH

SEQ ID NO: 1369 - protein, 7 aa, synthetic construct
IRHGYYD

SEQ ID NO: 1370 - protein, 7 aa, synthetic construct
INRGLST

SEQ ID NO: 1371 - protein, 7 aa, synthetic construct
ILRILPS

SEQ ID NO: 1372 - protein, 7 aa, synthetic construct
ILFHSSS

SEQ ID NO: 1373 - protein, 7 aa, synthetic construct
IKECSTP

SEQ ID NO: 1374 - protein, 7 aa, synthetic construct
IISWTGT

SEQ ID NO: 1375 - protein, 7 aa, synthetic construct
IFYSWCH

SEQ ID NO: 1376 - protein, 7 aa, synthetic construct
IERNGKS

SEQ ID NO: 1377 - protein, 7 aa, synthetic construct
HVSSRTA

SEQ ID NO: 1378 - protein, 7 aa, synthetic construct
HTDFIYS

SEQ ID NO: 1379 - protein, 7 aa, synthetic construct
HSHYTIA

SEQ ID NO: 1380 - protein, 7 aa, synthetic construct
GVMQWFQ

SEQ ID NO: 1381 - protein, 7 aa, synthetic construct
GTSMSSR

SEQ ID NO: 1382 - protein, 7 aa, synthetic construct
GRGPTQQ

SEQ ID NO: 1383 - protein, 7 aa, synthetic construct
GQERFWL

SEQ ID NO: 1384 - protein, 7 aa, synthetic construct
GPALHSV

SEQ ID NO: 1385 - protein, 7 aa, synthetic construct
GMQACYG

SEQ ID NO: 1386 - protein, 7 aa, synthetic construct
GMPCFGA

SEQ ID NO: 1387 - protein, 7 aa, synthetic construct
GLLSYTG

SEQ ID NO: 1388 - protein, 7 aa, synthetic construct
GKSTLFY

SEQ ID NO: 1389 - protein, 7 aa, synthetic construct
GGFSIYE

SEQ ID NO: 1390 - protein, 7 aa, synthetic construct
GARVWPV

SEQ ID NO: 1391 - protein, 7 aa, synthetic construct
FYLKGTS

SEQ ID NO: 1392 - protein, 7 aa, synthetic construct
FTRHQPS

SEQ ID NO: 1393 - protein, 7 aa, synthetic construct
FHDLRII

SEQ ID NO: 1394 - protein, 7 aa, synthetic construct
FFNIPFS

SEQ ID NO: 1395 - protein, 7 aa, synthetic construct
ELFHVKK

SEQ ID NO: 1396 - protein, 7 aa, synthetic construct
DWTHHSS

SEQ ID NO: 1397 - protein, 7 aa, synthetic construct
DPNCQRN

SEQ ID NO: 1398 - protein, 7 aa, synthetic construct
DITTCNY

SEQ ID NO: 1399 - protein, 7 aa, synthetic construct
DCSPFPP

SEQ ID NO: 1400 - protein, 7 aa, synthetic construct
CWVTRIQ

SEQ ID NO: 1401 - protein, 7 aa, synthetic construct
CVRSQNT

SEQ ID NO: 1402 - protein, 7 aa, synthetic construct
CQALGIM

SEQ ID NO: 1403 - protein, 7 aa, synthetic construct
CLSRSYI

SEQ ID NO: 1404 - protein, 7 aa, synthetic construct
CLGDPRL

SEQ ID NO: 1405 - protein, 7 aa, synthetic construct
AYLLHTP

SEQ ID NO: 1406 - protein, 7 aa, synthetic construct
ARQCVVD

SEQ ID NO: 1407 - protein, 7 aa, synthetic construct
AMPFMPG

SEQ ID NO: 1408 - protein, 7 aa, synthetic construct
ADYLLVL

SEQ ID NO: 1409 - protein, 7 aa, synthetic construct
KVCLCMP

SEQ ID NO: 1410 - protein, 7 aa, synthetic construct
TSPVGTL

SEQ ID NO: 1411 - protein, 7 aa, synthetic construct
SRLKLGD

SEQ ID NO: 1412 - protein, 7 aa, synthetic construct
GNRVTAP

SEQ ID NO: 1413 - protein, 7 aa, synthetic construct
NHASEPL

SEQ ID NO: 1414 - protein, 7 aa, synthetic construct
NRTGLPI

SEQ ID NO: 1415 - protein, 7 aa, synthetic construct
ENRIERQ

SEQ ID NO: 1416 - protein, 7 aa, synthetic construct
LSRQSVS

SEQ ID NO: 1417 - protein, 7 aa, synthetic construct
VKPTGSP

SEQ ID NO: 1418 - protein, 7 aa, synthetic construct
ASRSILA

SEQ ID NO: 1419 - protein, 7 aa, synthetic construct
QSRTSTP

SEQ ID NO: 1420 - protein, 7 aa, synthetic construct
STQRLQA

SEQ ID NO: 1421 - protein, 7 aa, synthetic construct
NSSKNLT

SEQ ID NO: 1422 - protein, 7 aa, synthetic construct
SSRMTSP

SEQ ID NO: 1423 - protein, 7 aa, synthetic construct
LTSNKLT

SEQ ID NO: 1424 - protein, 7 aa, synthetic construct
PNSAKAE

SEQ ID NO: 1425 - protein, 7 aa, synthetic construct
NTTKSPA

SEQ ID NO: 1426 - protein, 7 aa, synthetic construct
NVTKIPS

SEQ ID NO: 1427 - protein, 7 aa, synthetic construct
MNSARAD

SEQ ID NO: 1428 - protein, 7 aa, synthetic construct
SNQTAPS

SEQ ID NO: 1429 - protein, 7 aa, synthetic construct
NSRQQTP

SEQ ID NO: 1430 - protein, 7 aa, synthetic construct
SNEVRQG

SEQ ID NO: 1431 - protein, 7 aa, synthetic construct
NTARLQI

SEQ ID NO: 1432 - protein, 7 aa, synthetic construct
SGPSSDP

SEQ ID NO: 1433 - protein, 7 aa, synthetic construct
MVKALSQ

SEQ ID NO: 1434 - protein, 7 aa, synthetic construct
TTKAPST

SEQ ID NO: 1435 - protein, 7 aa, synthetic construct
TPTPQHQ

SEQ ID NO: 1436 - protein, 7 aa, synthetic construct
AQNRSPS

SEQ ID NO: 1437 - protein, 7 aa, synthetic construct
AGKAQMA

SEQ ID NO: 1438 - protein, 7 aa, synthetic construct
ATRQPPV

SEQ ID NO: 1439 - protein, 7 aa, synthetic construct
VLPGLNA

SEQ ID NO: 1440 - protein, 7 aa, synthetic construct
SVKMSPN

SEQ ID NO: 1441 - protein, 7 aa, synthetic construct
TSKTIQP

SEQ ID NO: 1442 - protein, 7 aa, synthetic construct
ETKRSSE

SEQ ID NO: 1443 - protein, 7 aa, synthetic construct
IRSIPPP

SEQ ID NO: 1444 - protein, 7 aa, synthetic construct
QNSVRQD

SEQ ID NO: 1445 - protein, 7 aa, synthetic construct
NLEQLRA

SEQ ID NO: 1446 - protein, 7 aa, synthetic construct
SGNLGRE

SEQ ID NO: 1447 - protein, 7 aa, synthetic construct
SRSTIPA

SEQ ID NO: 1448 - protein, 7 aa, synthetic construct
TSRQVSP

SEQ ID NO: 1449 - protein, 7 aa, synthetic construct
TNKIVPS

SEQ ID NO: 1450 - protein, 7 aa, synthetic construct
SIKQPGQ

SEQ ID NO: 1451 - protein, 7 aa, synthetic construct
STRGIVA

SEQ ID NO: 1452 - protein, 7 aa, synthetic construct
SIKSAQY

SEQ ID NO: 1453 - protein, 7 aa, synthetic construct
TTRIAQP

SEQ ID NO: 1454 - protein, 7 aa, synthetic construct
TIRVGGP

SEQ ID NO: 1455 - protein, 7 aa, synthetic construct
SVRSMTA

SEQ ID NO: 1456 - protein, 7 aa, synthetic construct
LATKIPP

SEQ ID NO: 1457 - protein, 7 aa, synthetic construct
NVTKIPA

SEQ ID NO: 1458 - protein, 7 aa, synthetic construct
TLPIGAQ

SEQ ID NO: 1459 - protein, 7 aa, synthetic construct
LGASKLP

SEQ ID NO: 1460 - protein, 7 aa, synthetic construct
QSKAGMS

SEQ ID NO: 1461 - protein, 7 aa, synthetic construct
FTRQEPT

SEQ ID NO: 1462 - protein, 7 aa, synthetic construct
DNTSTRS

SEQ ID NO: 1463 - protein, 7 aa, synthetic construct
TLSNKVS

SEQ ID NO: 1464 - protein, 7 aa, synthetic construct
MNTSRQD

SEQ ID NO: 1465 - protein, 7 aa, synthetic construct
LGHQKPA

SEQ ID NO: 1466 - protein, 7 aa, synthetic construct
IVPGLNQ

SEQ ID NO: 1467 - protein, 7 aa, synthetic construct
PPTHTQD

SEQ ID NO: 1468 - protein, 7 aa, synthetic construct
MTNRTQI

SEQ ID NO: 1469 - protein, 7 aa, synthetic construct
TSRAQSP

SEQ ID NO: 1470 - protein, 7 aa, synthetic construct
TNSAKGD

SEQ ID NO: 1471 - protein, 7 aa, synthetic construct
QAPLKQS

SEQ ID NO: 1472 - protein, 7 aa, synthetic construct
NVTKITA

SEQ ID NO: 1473 - protein, 7 aa, synthetic construct
FGVTRSP

SEQ ID NO: 1474 - protein, 7 aa, synthetic construct
NASKSPV

SEQ ID NO: 1475 - protein, 7 aa, synthetic construct
VNTVRGD

SEQ ID NO: 1476 - protein, 7 aa, synthetic construct
LNETSRT

SEQ ID NO: 1477 - protein, 7 aa, synthetic construct
ETRARLE

SEQ ID NO: 1478 - protein, 7 aa, synthetic construct
NVTKIPA

SEQ ID NO: 1479 - protein, 7 aa, synthetic construct
NLTKIPA

SEQ ID NO: 1480 - protein, 7 aa, synthetic construct
STKATSP

SEQ ID NO: 1481 - protein, 7 aa, synthetic construct
NVSKIPA

SEQ ID NO: 1482 - protein, 7 aa, synthetic construct
NVIKSSV

SEQ ID NO: 1483 - protein, 7 aa, synthetic construct
NRTSPMV

SEQ ID NO: 1484 - protein, 7 aa, synthetic construct
NQSKLNV

SEQ ID NO: 1485 - protein, 7 aa, synthetic construct
NVTKNPA

SEQ ID NO: 1486 - protein, 7 aa, synthetic construct
NVTKIQA

SEQ ID NO: 1487 - protein, 7 aa, synthetic construct
SVKLPAT

SEQ ID NO: 1488 - protein, 7 aa, synthetic construct
SIKSPGF

SEQ ID NO: 1489 - protein, 7 aa, synthetic construct
NAVKIPP

SEQ ID NO: 1490 - protein, 7 aa, synthetic construct
YNQTSRD

SEQ ID NO: 1491 - protein, 7 aa, synthetic construct
QENVIRS

SEQ ID NO: 1492 - protein, 7 aa, synthetic construct
SKPQSSP

SEQ ID NO: 1493 - protein, 7 aa, synthetic construct
RPDARLA

SEQ ID NO: 1494 - protein, 7 aa, synthetic construct
NVTKIPP

SEQ ID NO: 1495 - protein, 7 aa, synthetic construct
VDKAKLA

SEQ ID NO: 1496 - protein, 7 aa, synthetic construct
LVPGIQA

SEQ ID NO: 1497 - protein, 7 aa, synthetic construct
RTTTIPP

SEQ ID NO: 1498 - protein, 7 aa, synthetic construct
MSNLPPP

SEQ ID NO: 1499 - protein, 7 aa, synthetic construct
ENRIRQE

SEQ ID NO: 1500 - protein, 7 aa, synthetic construct
FNDTRLS

SEQ ID NO: 1501 - protein, 7 aa, synthetic construct
TNRTQSV

SEQ ID NO: 1502 - protein, 7 aa, synthetic construct
NSDTGRP

SEQ ID NO: 1503 - protein, 7 aa, synthetic construct
NFTKIPA

SEQ ID NO: 1504 - protein, 7 aa, synthetic construct
NIQRIPA

SEQ ID NO: 1505 - protein, 7 aa, synthetic construct
IVAGLDQ

SEQ ID NO: 1506 - protein, 7 aa, synthetic construct
VVSGLDA

SEQ ID NO: 1507 - protein, 7 aa, synthetic construct
VSAYMHA

SEQ ID NO: 1508 - protein, 7 aa, synthetic construct
NTAKALP

SEQ ID NO: 1509 - protein, 7 aa, synthetic construct
SIKVVQT

SEQ ID NO: 1510 - protein, 7 aa, synthetic construct
NYRTGTP

SEQ ID NO: 1511 - protein, 7 aa, synthetic construct
SVVRSSP

SEQ ID NO: 1512 - protein, 7 aa, synthetic construct
SNKTAPS

SEQ ID NO: 1513 - protein, 7 aa, synthetic construct
STKRTDT

SEQ ID NO: 1514 - protein, 7 aa, synthetic construct
GTRGLSP

SEQ ID NO: 1515 - protein, 7 aa, synthetic construct
SNQRILP

SEQ ID NO: 1516 - protein, 7 aa, synthetic construct
STKAPQP

SEQ ID NO: 1517 - protein, 7 aa, synthetic construct
RTSVGSP

SEQ ID NO: 1518 - protein, 7 aa, synthetic construct
TLSKIPA

SEQ ID NO: 1519 - protein, 7 aa, synthetic construct
AAPLRLS

SEQ ID NO: 1520 - protein, 7 aa, synthetic construct
AGVKTTP

SEQ ID NO: 1521 - protein, 7 aa, synthetic construct
TGSLESL

SEQ ID NO: 1522 - protein, 7 aa, synthetic construct
LGKVGSP

SEQ ID NO: 1523 - protein, 7 aa, synthetic construct
IPTLSAQ

SEQ ID NO: 1524 - protein, 7 aa, synthetic construct
YFGSGRE

SEQ ID NO: 1525 - protein, 7 aa, synthetic construct
AGKSQMA

SEQ ID NO: 1526 - protein, 7 aa, synthetic construct
VVPGLSY

SEQ ID NO: 1527 - protein, 7 aa, synthetic construct
QTRTNLP

SEQ ID NO: 1528 - protein, 7 aa, synthetic construct
KPPTHSP

SEQ ID NO: 1529 - protein, 7 aa, synthetic construct
STKDKNF

SEQ ID NO: 1530 - protein, 7 aa, synthetic construct
LVKSLQS

SEQ ID NO: 1531 - protein, 7 aa, synthetic construct
SVRLTSP

SEQ ID NO: 1532 - protein, 7 aa, synthetic construct
INGQKLS

SEQ ID NO: 1533 - protein, 7 aa, synthetic construct
SDKRASP

SEQ ID NO: 1534 - protein, 7 aa, synthetic construct
LRPTGAP

SEQ ID NO: 1535 - protein, 7 aa, synthetic construct
FTVSPPP

SEQ ID NO: 1536 - protein, 7 aa, synthetic construct
STKSQQM

SEQ ID NO: 1537 - protein, 7 aa, synthetic construct
MNHIRNE

SEQ ID NO: 1538 - protein, 7 aa, synthetic construct
NQIKLGM

SEQ ID NO: 1539 - protein, 7 aa, synthetic construct
LVPGLSY

SEQ ID NO: 1540 - protein, 7 aa, synthetic construct
STKKDSI

SEQ ID NO: 1541 - protein, 7 aa, synthetic construct
VLKNTTA

SEQ ID NO: 1542 - protein, 7 aa, synthetic construct
LVESLRA

SEQ ID NO: 1543 - protein, 7 aa, synthetic construct
TNQTRSE

SEQ ID NO: 1544 - protein, 7 aa, synthetic construct
SANVVLR

SEQ ID NO: 1545 - protein, 7 aa, synthetic construct
TLGKMQS

SEQ ID NO: 1546 - protein, 7 aa, synthetic construct
LVPGIPA

SEQ ID NO: 1547 - protein, 7 aa, synthetic construct
LTKALQG

SEQ ID NO: 1548 - protein, 7 aa, synthetic construct
TPAFKTP

SEQ ID NO: 1549 - protein, 7 aa, synthetic construct
IGSGSEH

SEQ ID NO: 1550 - protein, 7 aa, synthetic construct
VMRNTIA

SEQ ID NO: 1551 - protein, 7 aa, synthetic construct
LNKTQGF

SEQ ID NO: 1552 - protein, 7 aa, synthetic construct
PDISGQT

SEQ ID NO: 1553 - protein, 7 aa, synthetic construct
SDRAKSP

SEQ ID NO: 1554 - protein, 7 aa, synthetic construct
ATKIIQS

SEQ ID NO: 1555 - protein, 7 aa, synthetic construct
SSRQQPS

SEQ ID NO: 1556 - protein, 7 aa, synthetic construct
TITNRAE

SEQ ID NO: 1557 - protein, 7 aa, synthetic construct
SSSNLMK

SEQ ID NO: 1558 - protein, 7 aa, synthetic construct
VNQSRSD

SEQ ID NO: 1559 - protein, 7 aa, synthetic construct
SNQVQRD

SEQ ID NO: 1560 - protein, 7 aa, synthetic construct
GRPTSLP

SEQ ID NO: 1561 - protein, 7 aa, synthetic construct
LTQRSPS

SEQ ID NO: 1562 - protein, 7 aa, synthetic construct
SDNAVTV

SEQ ID NO: 1563 - protein, 7 aa, synthetic construct
VLRKLPE

SEQ ID NO: 1564 - protein, 7 aa, synthetic construct
SRVGLNP

SEQ ID NO: 1565 - protein, 7 aa, synthetic construct
TTSPLKA

SEQ ID NO: 1566 - protein, 7 aa, synthetic construct
NAAKAMI

SEQ ID NO: 1567 - protein, 7 aa, synthetic construct
STKVVLS

SEQ ID NO: 1568 - protein, 7 aa, synthetic construct
IVAGLTS

SEQ ID NO: 1569 - protein, 7 aa, synthetic construct
PMRALQP

SEQ ID NO: 1570 - protein, 7 aa, synthetic construct
DNATLRP

SEQ ID NO: 1571 - protein, 7 aa, synthetic construct
NVTKILA

SEQ ID NO: 1572 - protein, 7 aa, synthetic construct
ATKIIQT

SEQ ID NO: 1573 - protein, 7 aa, synthetic construct
NNRSLPI

SEQ ID NO: 1574 - protein, 7 aa, synthetic construct
ASRLGTA

SEQ ID NO: 1575 - protein, 7 aa, synthetic construct
QVRTISP

SEQ ID NO: 1576 - protein, 7 aa, synthetic construct
LLRNTTA

SEQ ID NO: 1577 - protein, 7 aa, synthetic construct
TTQRLQA

SEQ ID NO: 1578 - protein, 7 aa, synthetic construct
LIKNATS

SEQ ID NO: 1579 - protein, 7 aa, synthetic construct
QNRTLPL

SEQ ID NO: 1580 - protein, 7 aa, synthetic construct
ITKNTTV

SEQ ID NO: 1581 - protein, 7 aa, synthetic construct
STQRLQV

SEQ ID NO: 1582 - protein, 7 aa, synthetic construct
NTRGRDD

SEQ ID NO: 1583 - protein, 7 aa, synthetic construct
RTKVSDP

SEQ ID NO: 1584 - protein, 7 aa, synthetic construct
LGIPKSP

SEQ ID NO: 1585 - protein, 7 aa, synthetic construct
VTPYKTG

SEQ ID NO: 1586 - protein, 7 aa, synthetic construct
VQKPHPP

SEQ ID NO: 1587 - protein, 7 aa, synthetic construct
TTRSSIP

SEQ ID NO: 1588 - protein, 7 aa, synthetic construct
TTRIPPS

SEQ ID NO: 1589 - protein, 7 aa, synthetic construct
TTRGIPS

SEQ ID NO: 1590 - protein, 7 aa, synthetic construct
TPQVRMS

SEQ ID NO: 1591 - protein, 7 aa, synthetic construct
STQRLQS

SEQ ID NO: 1592 - protein, 7 aa, synthetic construct
SPQPSRA

SEQ ID NO: 1593 - protein, 7 aa, synthetic construct
SLRDRSM

SEQ ID NO: 1594 - protein, 7 aa, synthetic construct
SIVRSAV

SEQ ID NO: 1595 - protein, 7 aa, synthetic construct
SGQTPRP

SEQ ID NO: 1596 - protein, 7 aa, synthetic construct
SGPMKTS

SEQ ID NO: 1597 - protein, 7 aa, synthetic construct
RTSQPPP

SEQ ID NO: 1598 - protein, 7 aa, synthetic construct
RGRPSDP

SEQ ID NO: 1599 - protein, 7 aa, synthetic construct
QRTYAPP

SEQ ID NO: 1600 - protein, 7 aa, synthetic construct
QIRTMGP

SEQ ID NO: 1601 - protein, 7 aa, synthetic construct
QIRLGGS

SEQ ID NO: 1602 - protein, 7 aa, synthetic construct
NVTAKAP

SEQ ID NO: 1603 - protein, 7 aa, synthetic construct
NSVPRSP

SEQ ID NO: 1604 - protein, 7 aa, synthetic construct
NMSRTIS

SEQ ID NO: 1605 - protein, 7 aa, synthetic construct
LTRQAGS

SEQ ID NO: 1606 - protein, 7 aa, synthetic construct
LEQSGRE

SEQ ID NO: 1607 - protein, 7 aa, synthetic construct
ISDSGRP

SEQ ID NO: 1608 - protein, 7 aa, synthetic construct
IKVSPPP

SEQ ID NO: 1609 - protein, 7 aa, synthetic construct
HFPSVLL

SEQ ID NO: 1610 - protein, 7 aa, synthetic construct
ASNRLTA

SEQ ID NO: 1611 - protein, 7 aa, synthetic construct
ANRSQSP

SEQ ID NO: 1612 - protein, 7 aa, synthetic construct
AISNLAK

SEQ ID NO: 1613 - protein, 7 aa, synthetic construct
VYTKLWM

SEQ ID NO: 1614 - protein, 7 aa, synthetic construct
CLSSSLN

SEQ ID NO: 1615 - protein, 7 aa, synthetic construct
RAHFLWN

SEQ ID NO: 1616 - protein, 7 aa, synthetic construct
APDFSYP

SEQ ID NO: 1617 - protein, 7 aa, synthetic construct
RPRVITL

SEQ ID NO: 1618 - protein, 7 aa, synthetic construct
MVYNLQM

SEQ ID NO: 1619 - protein, 7 aa, synthetic construct
SHVSKRS

SEQ ID NO: 1620 - protein, 7 aa, synthetic construct
SAIFTGP

SEQ ID NO: 1621 - protein, 7 aa, synthetic construct
SIQQGPR

SEQ ID NO: 1622 - protein, 7 aa, synthetic construct
ERSSQSS

SEQ ID NO: 1623 - protein, 7 aa, synthetic construct
LQSFAIQ

SEQ ID NO: 1624 - protein, 7 aa, synthetic construct
LTETVES

SEQ ID NO: 1625 - protein, 7 aa, synthetic construct
LDYYSST

SEQ ID NO: 1626 - protein, 7 aa, synthetic construct
ISIMFGS

SEQ ID NO: 1627 - protein, 7 aa, synthetic construct
VKRLMLL

SEQ ID NO: 1628 - protein, 7 aa, synthetic construct
YLNEFMA

SEQ ID NO: 1629 - protein, 7 aa, synthetic construct
ILGSILT

SEQ ID NO: 1630 - protein, 7 aa, synthetic construct
RVMGSKL

SEQ ID NO: 1631 - protein, 7 aa, synthetic construct
IPRLTDV

SEQ ID NO: 1632 - protein, 7 aa, synthetic construct
HHRSIHH

SEQ ID NO: 1633 - protein, 7 aa, synthetic construct
DISSVMS

SEQ ID NO: 1634 - protein, 7 aa, synthetic construct
CEGWYGR

SEQ ID NO: 1635 - protein, 7 aa, synthetic construct
RWAPLNQ

SEQ ID NO: 1636 - protein, 7 aa, synthetic construct
PQSGSPE

SEQ ID NO: 1637 - protein, 7 aa, synthetic construct
QVSGQYQ

SEQ ID NO: 1638 - protein, 7 aa, synthetic construct
DLLLISS

SEQ ID NO: 1639 - protein, 7 aa, synthetic construct
GPVLMSS

SEQ ID NO: 1640 - protein, 7 aa, synthetic construct
CYNYMLV

SEQ ID NO: 1641 - protein, 7 aa, synthetic construct
TMPHIRL

SEQ ID NO: 1642 - protein, 7 aa, synthetic construct
SFNRRTL

SEQ ID NO: 1643 - protein, 7 aa, synthetic construct
FHNFPSP

SEQ ID NO: 1644 - protein, 7 aa, synthetic construct
SLQALEP

SEQ ID NO: 1645 - protein, 7 aa, synthetic construct
EGVCILA

SEQ ID NO: 1646 - protein, 7 aa, synthetic construct
LRDQKAK

SEQ ID NO: 1647 - protein, 7 aa, synthetic construct
PQASVLR

SEQ ID NO: 1648 - protein, 7 aa, synthetic construct
NGHGYYD

SEQ ID NO: 1649 - protein, 7 aa, synthetic construct
HMTSMPS

SEQ ID NO: 1650 - protein, 7 aa, synthetic construct
SQWHSSM

SEQ ID NO: 1651 - protein, 7 aa, synthetic construct
SQDSMQT

SEQ ID NO: 1652 - protein, 7 aa, synthetic construct
WYLWIRI

SEQ ID NO: 1653 - protein, 7 aa, synthetic construct
GYSLTTS

SEQ ID NO: 1654 - protein, 7 aa, synthetic construct
GVGHVHA

SEQ ID NO: 1655 - protein, 7 aa, synthetic construct
PWQLQVN

SEQ ID NO: 1656 - protein, 7 aa, synthetic construct
QYSLAAS

SEQ ID NO: 1657 - protein, 7 aa, synthetic construct
AMPTSQE

SEQ ID NO: 1658 - protein, 7 aa, synthetic construct
LGHYQPY

SEQ ID NO: 1659 - protein, 7 aa, synthetic construct
SRLKLRD

SEQ ID NO: 1660 - protein, 7 aa, synthetic construct
AMYSSSF

SEQ ID NO: 1661 - protein, 7 aa, synthetic construct
VRSSATT

SEQ ID NO: 1662 - protein, 7 aa, synthetic construct
GPALHSL

SEQ ID NO: 1663 - protein, 7 aa, synthetic construct
TGDSGPL

SEQ ID NO: 1664 - protein, 7 aa, synthetic construct
SVTFGSP

SEQ ID NO: 1665 - protein, 7 aa, synthetic construct
TVLKRTL

SEQ ID NO: 1666 - protein, 7 aa, synthetic construct
NPQDTLG

SEQ ID NO: 1667 - protein, 7 aa, synthetic construct
SSTLKPG

SEQ ID NO: 1668 - protein, 7 aa, synthetic construct
PLKQEVP

SEQ ID NO: 1669 - protein, 7 aa, synthetic construct
PLPTSPL

SEQ ID NO: 1670 - protein, 7 aa, synthetic construct
AGSLGLK

SEQ ID NO: 1671 - protein, 7 aa, synthetic construct
KGTPAEE

SEQ ID NO: 1672 - protein, 7 aa, synthetic construct
QEGVGMS

SEQ ID NO: 1673 - protein, 7 aa, synthetic construct
SRSPSTQ

SEQ ID NO: 1674 - protein, 7 aa, synthetic construct
EDSPSQM

SEQ ID NO: 1675 - protein, 7 aa, synthetic construct
ISTLQPI

SEQ ID NO: 1676 - protein, 7 aa, synthetic construct
ALTLSYV

SEQ ID NO: 1677 - protein, 7 aa, synthetic construct
VVSLQFE

SEQ ID NO: 1678 - protein, 7 aa, synthetic construct
SPSTPIL

SEQ ID NO: 1679 - protein, 7 aa, synthetic construct
DEPPQMS

SEQ ID NO: 1680 - protein, 7 aa, synthetic construct
IDNAEPR

SEQ ID NO: 1681 - protein, 7 aa, synthetic construct
TESTAMS

SEQ ID NO: 1682 - protein, 7 aa, synthetic construct
KLYAITV

SEQ ID NO: 1683 - protein, 7 aa, synthetic construct
LPSNLSE

SEQ ID NO: 1684 - protein, 7 aa, synthetic construct
HAAPGMC

SEQ ID NO: 1685 - protein, 7 aa, synthetic construct
LSLIATS

SEQ ID NO: 1686 - protein, 7 aa, synthetic construct
TQTDSAG

SEQ ID NO: 1687 - protein, 7 aa, synthetic construct
KVNTRVN

SEQ ID NO: 1688 - protein, 7 aa, synthetic construct
YPLSPET

SEQ ID NO: 1689 - protein, 7 aa, synthetic construct
THSTMYP

SEQ ID NO: 1690 - protein, 7 aa, synthetic construct
AVVFSDS

SEQ ID NO: 1691 - protein, 7 aa, synthetic construct
PDGPQHS

SEQ ID NO: 1692 - protein, 7 aa, synthetic construct
RYRAGPQ

SEQ ID NO: 1693 - protein, 7 aa, synthetic construct
TQDVMNL

SEQ ID NO: 1694 - protein, 7 aa, synthetic construct
ETKLSSE

SEQ ID NO: 1695 - protein, 7 aa, synthetic construct
AYRGFAH

SEQ ID NO: 1696 - protein, 7 aa, synthetic construct
VLGTVLE

SEQ ID NO: 1697 - protein, 7 aa, synthetic construct
TSQEQGS

SEQ ID NO: 1698 - protein, 7 aa, synthetic construct
LPPNILS

SEQ ID NO: 1699 - protein, 7 aa, synthetic construct
AHCDTYQ

SEQ ID NO: 1700 - protein, 7 aa, synthetic construct
KPTHKSA

SEQ ID NO: 1701 - protein, 7 aa, synthetic construct
SDSGLLP

SEQ ID NO: 1702 - protein, 7 aa, synthetic construct
SLGVALV

SEQ ID NO: 1703 - protein, 7 aa, synthetic construct
ERSALFQ

SEQ ID NO: 1704 - protein, 7 aa, synthetic construct
RPRPPNP

SEQ ID NO: 1705 - protein, 7 aa, synthetic construct
AMQEGLD

SEQ ID NO: 1706 - protein, 7 aa, synthetic construct
CPAQIIP

SEQ ID NO: 1707 - protein, 7 aa, synthetic construct
MMQYGYE

SEQ ID NO: 1708 - protein, 7 aa, synthetic construct
VLGHMLA

SEQ ID NO: 1709 - protein, 7 aa, synthetic construct
RAPPLTG

SEQ ID NO: 1710 - protein, 7 aa, synthetic construct
LSERGDS

SEQ ID NO: 1711 - protein, 7 aa, synthetic construct
PYTGDHP

SEQ ID NO: 1712 - protein, 7 aa, synthetic construct
INERLEV

SEQ ID NO: 1713 - protein, 7 aa, synthetic construct
VFSGLAA

SEQ ID NO: 1714 - protein, 7 aa, synthetic construct
QNTAVPT

SEQ ID NO: 1715 - protein, 7 aa, synthetic construct
IDAHTLG

SEQ ID NO: 1716 - protein, 7 aa, synthetic construct
SLGVAQE

SEQ ID NO: 1717 - protein, 7 aa, synthetic construct
LGHESLG

SEQ ID NO: 1718 - protein, 7 aa, synthetic construct
VLGHMLA

SEQ ID NO: 1719 - protein, 7 aa, synthetic construct
MLDLHSP

SEQ ID NO: 1720 - protein, 7 aa, synthetic construct
GLYCTDA

SEQ ID NO: 1721 - protein, 7 aa, synthetic construct
LSLATWV

SEQ ID NO: 1722 - protein, 7 aa, synthetic construct
HSHLTPE

SEQ ID NO: 1723 - protein, 7 aa, synthetic construct
VHQAIEI

SEQ ID NO: 1724 - protein, 7 aa, synthetic construct
VTGIHPS

SEQ ID NO: 1725 - protein, 7 aa, synthetic construct
ETAYMHA

SEQ ID NO: 1726 - protein, 7 aa, synthetic construct
ILPTSSL

SEQ ID NO: 1727 - protein, 7 aa, synthetic construct
PSRMMVS

SEQ ID NO: 1728 - protein, 7 aa, synthetic construct
PSDSGAS

SEQ ID NO: 1729 - protein, 7 aa, synthetic construct
MLGSCPS

SEQ ID NO: 1730 - protein, 7 aa, synthetic construct
FCMIFVH

SEQ ID NO: 1731 - protein, 7 aa, synthetic construct
LLCTALT

SEQ ID NO: 1732 - protein, 7 aa, synthetic construct
NLTVSVQ

SEQ ID NO: 1733 - protein, 7 aa, synthetic construct
NSHRETS

SEQ ID NO: 1734 - protein, 7 aa, synthetic construct
DIRGPVI

SEQ ID NO: 1735 - protein, 7 aa, synthetic construct
LRMSAFQ

SEQ ID NO: 1736 - protein, 7 aa, synthetic construct
SYYQTTQ

SEQ ID NO: 1737 - protein, 7 aa, synthetic construct
TIPASLM

SEQ ID NO: 1738 - protein, 7 aa, synthetic construct
CGITGSA

SEQ ID NO: 1739 - protein, 7 aa, synthetic construct
ISTIFMP

SEQ ID NO: 1740 - protein, 7 aa, synthetic construct
VYHPQLP

SEQ ID NO: 1741 - protein, 7 aa, synthetic construct
ASPLHPQ

SEQ ID NO: 1742 - protein, 7 aa, synthetic construct
QNGPLLL

SEQ ID NO: 1743 - protein, 7 aa, synthetic construct
SLGTQMV

SEQ ID NO: 1744 - protein, 7 aa, synthetic construct
RVFHIPM

SEQ ID NO: 1745 - protein, 7 aa, synthetic construct
YVGTEPA

SEQ ID NO: 1746 - protein, 7 aa, synthetic construct
VLGHMLV

SEQ ID NO: 1747 - protein, 7 aa, synthetic construct
LEQSGRE

SEQ ID NO: 1748 - protein, 7 aa, synthetic construct
PDTSGSP

SEQ ID NO: 1749 - protein, 7 aa, synthetic construct
MTPPVSS

SEQ ID NO: 1750 - protein, 7 aa, synthetic construct
TCAILNT

SEQ ID NO: 1751 - protein, 7 aa, synthetic construct
QMHASPS

SEQ ID NO: 1752 - protein, 7 aa, synthetic construct
SSLNLDP

SEQ ID NO: 1753 - protein, 7 aa, synthetic construct
SLSQSAD

SEQ ID NO: 1754 - protein, 7 aa, synthetic construct
LSAPMYT

SEQ ID NO: 1755 - protein, 7 aa, synthetic construct
PLPYGSP

SEQ ID NO: 1756 - protein, 7 aa, synthetic construct
PNEIRLE

SEQ ID NO: 1757 - protein, 7 aa, synthetic construct
PALGVDF

SEQ ID NO: 1758 - protein, 7 aa, synthetic construct
TRGPNST

SEQ ID NO: 1759 - protein, 7 aa, synthetic construct
RLGYQSV

SEQ ID NO: 1760 - protein, 7 aa, synthetic construct
TLVSTTE

SEQ ID NO: 1761 - protein, 7 aa, synthetic construct
ALQPQLS

SEQ ID NO: 1762 - protein, 7 aa, synthetic construct
TFSSGSE

SEQ ID NO: 1763 - protein, 7 aa, synthetic construct
LSQYILS

SEQ ID NO: 1764 - protein, 7 aa, synthetic construct
PSPQDFS

SEQ ID NO: 1765 - protein, 7 aa, synthetic construct
KLSAISV

SEQ ID NO: 1766 - protein, 7 aa, synthetic construct
TSVLTLT

SEQ ID NO: 1767 - protein, 7 aa, synthetic construct
VYTKLRK

SEQ ID NO: 1768 - protein, 7 aa, synthetic construct
NQTSIPL

SEQ ID NO: 1769 - protein, 7 aa, synthetic construct
RMPSTQT

SEQ ID NO: 1770 - protein, 7 aa, synthetic construct
GSSPSAT

SEQ ID NO: 1771 - protein, 7 aa, synthetic construct
NMETLVP

SEQ ID NO: 1772 - protein, 7 aa, synthetic construct
GTLVHEP

SEQ ID NO: 1773 - protein, 7 aa, synthetic construct
SATPLLP

SEQ ID NO: 1774 - protein, 7 aa, synthetic construct
PASFIQY

SEQ ID NO: 1775 - protein, 7 aa, synthetic construct
VLGHMLA

SEQ ID NO: 1776 - protein, 7 aa, synthetic construct
SDGHHSA

SEQ ID NO: 1777 - protein, 7 aa, synthetic construct
TETYINS

SEQ ID NO: 1778 - protein, 7 aa, synthetic construct
MAETDYV

SEQ ID NO: 1779 - protein, 7 aa, synthetic construct
LNTVVLA

SEQ ID NO: 1780 - protein, 7 aa, synthetic construct
GLSSTLE

SEQ ID NO: 1781 - protein, 7 aa, synthetic construct
TNTTHGD

SEQ ID NO: 1782 - protein, 7 aa, synthetic construct
SPCRLIR

SEQ ID NO: 1783 - protein, 7 aa, synthetic construct
NLQSLPS

SEQ ID NO: 1784 - protein, 7 aa, synthetic construct
LPQTLLA

SEQ ID NO: 1785 - protein, 7 aa, synthetic construct
TLGSLPL

SEQ ID NO: 1786 - protein, 7 aa, synthetic construct
LVESVHS

SEQ ID NO: 1787 - protein, 7 aa, synthetic construct
LLRTALT

SEQ ID NO: 1788 - protein, 7 aa, synthetic construct
SPPYNVS

SEQ ID NO: 1789 - protein, 7 aa, synthetic construct
LSVSYQQ

SEQ ID NO: 1790 - protein, 7 aa, synthetic construct
TFGQSLD

SEQ ID NO: 1791 - protein, 7 aa, synthetic construct
QNVLTQN

SEQ ID NO: 1792 - protein, 7 aa, synthetic construct
TLSHSRT

SEQ ID NO: 1793 - protein, 7 aa, synthetic construct
LELSHQG

SEQ ID NO: 1794 - protein, 7 aa, synthetic construct
GDYEHNP

SEQ ID NO: 1795 - protein, 7 aa, synthetic construct
LHFSGVY

SEQ ID NO: 1796 - protein, 7 aa, synthetic construct
AIRGEAP

SEQ ID NO: 1797 - protein, 7 aa, synthetic construct
NSVHLPQ

SEQ ID NO: 1798 - protein, 7 aa, synthetic construct
DGSSYPI

SEQ ID NO: 1799 - protein, 7 aa, synthetic construct
AQAQLSE

SEQ ID NO: 1800 - protein, 7 aa, synthetic construct
LTDQFPQ

SEQ ID NO: 1801 - protein, 7 aa, synthetic construct
RHTTQPS

SEQ ID NO: 1802 - protein, 7 aa, synthetic construct
SASYMAD

SEQ ID NO: 1803 - protein, 7 aa, synthetic construct
SNRNEPP

SEQ ID NO: 1804 - protein, 7 aa, synthetic construct
LLVLGSG

SEQ ID NO: 1805 - protein, 7 aa, synthetic construct
LGHMGTE

SEQ ID NO: 1806 - protein, 7 aa, synthetic construct
MALSMSE

SEQ ID NO: 1807 - protein, 7 aa, synthetic construct
FMAHMTS

SEQ ID NO: 1808 - protein, 7 aa, synthetic construct
FVSMTTE

SEQ ID NO: 1809 - protein, 7 aa, synthetic construct
GQRGLLG

SEQ ID NO: 1810 - protein, 7 aa, synthetic construct
VHETDPC

SEQ ID NO: 1811 - protein, 7 aa, synthetic construct
DSLGSSS

SEQ ID NO: 1812 - protein, 7 aa, synthetic construct
PGRMNFS

SEQ ID NO: 1813 - protein, 7 aa, synthetic construct
FGPQTQA

SEQ ID NO: 1814 - protein, 7 aa, synthetic construct
NALSEHY

SEQ ID NO: 1815 - protein, 7 aa, synthetic construct
MPKSDAP

SEQ ID NO: 1816 - protein, 7 aa, synthetic construct
PALYIVL

SEQ ID NO: 1817 - protein, 7 aa, synthetic construct
YTSNLTA

SEQ ID NO: 1818 - protein, 7 aa, synthetic construct
TVKPIST

SEQ ID NO: 1819 - protein, 7 aa, synthetic construct
NSAQFPI

SEQ ID NO: 1820 - protein, 7 aa, synthetic construct
NLRTGLT

SEQ ID NO: 1821 - protein, 7 aa, synthetic construct
MPTISHS

SEQ ID NO: 1822 - protein, 7 aa, synthetic construct
YTSNLMM

SEQ ID NO: 1823 - protein, 7 aa, synthetic construct
SHSSEFS

SEQ ID NO: 1824 - protein, 7 aa, synthetic construct
LTKMSPT

SEQ ID NO: 1825 - protein, 7 aa, synthetic construct
EHQSLLS

SEQ ID NO: 1826 - protein, 7 aa, synthetic construct
QPCGFPM

SEQ ID NO: 1827 - protein, 7 aa, synthetic construct
TADYSVP

SEQ ID NO: 1828 - protein, 7 aa, synthetic construct
TKSIPST

SEQ ID NO: 1829 - protein, 7 aa, synthetic construct
TPTSQDT

SEQ ID NO: 1830 - protein, 7 aa, synthetic construct
LPYLLYP

SEQ ID NO: 1831 - protein, 7 aa, synthetic construct
SVGHTFV

SEQ ID NO: 1832 - protein, 7 aa, synthetic construct
LGDKKVK

SEQ ID NO: 1833 - protein, 7 aa, synthetic construct
SGPLISE

SEQ ID NO: 1834 - protein, 7 aa, synthetic construct
TTSNEIT

SEQ ID NO: 1835 - protein, 7 aa, synthetic construct
KPISTLS

SEQ ID NO: 1836 - protein, 7 aa, synthetic construct
GLERSES

SEQ ID NO: 1837 - protein, 7 aa, synthetic construct
AVMQYVP

SEQ ID NO: 1838 - protein, 7 aa, synthetic construct
GMPGTHQ

SEQ ID NO: 1839 - protein, 7 aa, synthetic construct
LPHTQPL

SEQ ID NO: 1840 - protein, 7 aa, synthetic construct
STPRLDS

SEQ ID NO: 1841 - protein, 7 aa, synthetic construct
MNTQLSP

SEQ ID NO: 1842 - protein, 7 aa, synthetic construct
YTSVQLN

SEQ ID NO: 1843 - protein, 7 aa, synthetic construct
NPREGKS

SEQ ID NO: 1844 - protein, 7 aa, synthetic construct
VMPQVLS

SEQ ID NO: 1845 - protein, 7 aa, synthetic construct
VLIVGAQ

SEQ ID NO: 1846 - protein, 7 aa, synthetic construct
TNHTFTS

SEQ ID NO: 1847 - protein, 7 aa, synthetic construct
AHIASTV

SEQ ID NO: 1848 - protein, 7 aa, synthetic construct
TMPPTQP

SEQ ID NO: 1849 - protein, 7 aa, synthetic construct
PPVGINM

SEQ ID NO: 1850 - protein, 7 aa, synthetic construct
NMTASPL

SEQ ID NO: 1851 - protein, 7 aa, synthetic construct
SVKTAVM

SEQ ID NO: 1852 - protein, 7 aa, synthetic construct
PTEHVPP

SEQ ID NO: 1853 - protein, 7 aa, synthetic construct
STKSTSS

SEQ ID NO: 1854 - protein, 7 aa, synthetic construct
AKIQRVA

SEQ ID NO: 1855 - protein, 7 aa, synthetic construct
NIVIVVT

SEQ ID NO: 1856 - protein, 7 aa, synthetic construct
LLAPNLS

SEQ ID NO: 1857 - protein, 7 aa, synthetic construct
DTLSLGS

SEQ ID NO: 1858 - protein, 7 aa, synthetic construct
GNTTSLS

SEQ ID NO: 1859 - protein, 7 aa, synthetic construct
MPNTILS

SEQ ID NO: 1860 - protein, 7 aa, synthetic construct
GIQLTSS

SEQ ID NO: 1861 - protein, 7 aa, synthetic construct
TPQSSMF

SEQ ID NO: 1862 - protein, 7 aa, synthetic construct
LTSGLAG

SEQ ID NO: 1863 - protein, 7 aa, synthetic construct
LDRTTLP

SEQ ID NO: 1864 - protein, 7 aa, synthetic construct
VLREQPP

SEQ ID NO: 1865 - protein, 7 aa, synthetic construct
HSLMHHS

SEQ ID NO: 1866 - protein, 7 aa, synthetic construct
WEGHSIF

SEQ ID NO: 1867 - protein, 7 aa, synthetic construct
TGQIHPE

SEQ ID NO: 1868 - protein, 7 aa, synthetic construct
HLSSVIS

SEQ ID NO: 1869 - protein, 7 aa, synthetic construct
SMVVSTP

SEQ ID NO: 1870 - protein, 7 aa, synthetic construct
YPTSQPP

SEQ ID NO: 1871 - protein, 7 aa, synthetic construct
LTTIPPP

SEQ ID NO: 1872 - protein, 7 aa, synthetic construct
IKLYSQS

SEQ ID NO: 1873 - protein, 7 aa, synthetic construct
TPPLNMQ

SEQ ID NO: 1874 - protein, 7 aa, synthetic construct
CTPPSTH

SEQ ID NO: 1875 - protein, 7 aa, synthetic construct
LHRFQEP

SEQ ID NO: 1876 - protein, 7 aa, synthetic construct
FGYSMES

SEQ ID NO: 1877 - protein, 7 aa, synthetic construct
TLQTLAE

SEQ ID NO: 1878 - protein, 7 aa, synthetic construct
ILGQSSI

SEQ ID NO: 1879 - protein, 7 aa, synthetic construct
LSASMVS

SEQ ID NO: 1880 - protein, 7 aa, synthetic construct
SESMMHG

SEQ ID NO: 1881 - protein, 7 aa, synthetic construct
EILTPVI

SEQ ID NO: 1882 - protein, 7 aa, synthetic construct
SLQPSPL

SEQ ID NO: 1883 - protein, 7 aa, synthetic construct
LESYSGW

SEQ ID NO: 1884 - protein, 7 aa, synthetic construct
HDRNYTP

SEQ ID NO: 1885 - protein, 7 aa, synthetic construct
EGTAYSA

SEQ ID NO: 1886 - protein, 7 aa, synthetic construct
EAQPHTS

SEQ ID NO: 1887 - protein, 7 aa, synthetic construct
SERVPTR

SEQ ID NO: 1888 - protein, 7 aa, synthetic construct
TANIGGP

SEQ ID NO: 1889 - protein, 7 aa, synthetic construct
HHCLPGS

SEQ ID NO: 1890 - protein, 7 aa, synthetic construct
HLSSTEP

SEQ ID NO: 1891 - protein, 7 aa, synthetic construct
DQPISAP

SEQ ID NO: 1892 - protein, 7 aa, synthetic construct
TQKFHGL

SEQ ID NO: 1893 - protein, 7 aa, synthetic construct
WSTYMNS

SEQ ID NO: 1894 - protein, 7 aa, synthetic construct
MVNVDHA

SEQ ID NO: 1895 - protein, 7 aa, synthetic construct
APPTSTP

SEQ ID NO: 1896 - protein, 7 aa, synthetic construct
THGQTDH

SEQ ID NO: 1897 - protein, 7 aa, synthetic construct
IKIPSQG

SEQ ID NO: 1898 - protein, 7 aa, synthetic construct
GGSQQLE

SEQ ID NO: 1899 - protein, 7 aa, synthetic construct
EISNSFR

SEQ ID NO: 1900 - protein, 7 aa, synthetic construct
VAYQMSP

SEQ ID NO: 1901 - protein, 7 aa, synthetic construct
SRNASYD

SEQ ID NO: 1902 - protein, 7 aa, synthetic construct
ATVGTQP

SEQ ID NO: 1903 - protein, 7 aa, synthetic construct
STTLHYP

SEQ ID NO: 1904 - protein, 7 aa, synthetic construct
GTPPNFS

SEQ ID NO: 1905 - protein, 7 aa, synthetic construct
TQGSANV

SEQ ID NO: 1906 - protein, 7 aa, synthetic construct
ITIASLN

SEQ ID NO: 1907 - protein, 7 aa, synthetic construct
YSQDATA

SEQ ID NO: 1908 - protein, 7 aa, synthetic construct
KPVSAQA

SEQ ID NO: 1909 - protein, 7 aa, synthetic construct
IFAGLTS

SEQ ID NO: 1910 - protein, 7 aa, synthetic construct
LTAMLLS

SEQ ID NO: 1911 - protein, 7 aa, synthetic construct
KMGMRLS

SEQ ID NO: 1912 - protein, 7 aa, synthetic construct
TVRANQK

SEQ ID NO: 1913 - protein, 7 aa, synthetic construct
HAAGREE

SEQ ID NO: 1914 - protein, 7 aa, synthetic construct
ISLEIFR

SEQ ID NO: 1915 - protein, 7 aa, synthetic construct
RGSQGLS

SEQ ID NO: 1916 - protein, 7 aa, synthetic construct
LAEYPGQ

SEQ ID NO: 1917 - protein, 7 aa, synthetic construct
SQLGTVT

SEQ ID NO: 1918 - protein, 7 aa, synthetic construct
SMSASEP

SEQ ID NO: 1919 - protein, 7 aa, synthetic construct
TQAFSSD

SEQ ID NO: 1920 - protein, 7 aa, synthetic construct
KNNPQMT

SEQ ID NO: 1921 - protein, 7 aa, synthetic construct
SQTDTAG

SEQ ID NO: 1922 - protein, 7 aa, synthetic construct
LFPNASP

SEQ ID NO: 1923 - protein, 7 aa, synthetic construct
THTLSVE

SEQ ID NO: 1924 - protein, 7 aa, synthetic construct
GGGFGLE

SEQ ID NO: 1925 - protein, 7 aa, synthetic construct
LSRTIQP

SEQ ID NO: 1926 - protein, 7 aa, synthetic construct
NAASLLP

SEQ ID NO: 1927 - protein, 7 aa, synthetic construct
SSIGMPV

SEQ ID NO: 1928 - protein, 7 aa, synthetic construct
LGIASHE

SEQ ID NO: 1929 - protein, 7 aa, synthetic construct
ETSPHIN

SEQ ID NO: 1930 - protein, 7 aa, synthetic construct
VALQGGP

SEQ ID NO: 1931 - protein, 7 aa, synthetic construct
VPISNLA

SEQ ID NO: 1932 - protein, 7 aa, synthetic construct
FSRTMQD

SEQ ID NO: 1933 - protein, 7 aa, synthetic construct
SLQTQPQ

SEQ ID NO: 1934 - protein, 7 aa, synthetic construct
LAPAILS

SEQ ID NO: 1935 - protein, 7 aa, synthetic construct
LDMSSSL

SEQ ID NO: 1936 - protein, 7 aa, synthetic construct
QTNQSLT

SEQ ID NO: 1937 - protein, 7 aa, synthetic construct
FSSTGVP

SEQ ID NO: 1938 - protein, 7 aa, synthetic construct
SVQSLAS

SEQ ID NO: 1939 - protein, 7 aa, synthetic construct
EINRSTP

SEQ ID NO: 1940 - protein, 7 aa, synthetic construct
APTHQMS

SEQ ID NO: 1941 - protein, 7 aa, synthetic construct
NALLQCA

SEQ ID NO: 1942 - protein, 7 aa, synthetic construct
SKISIAG

SEQ ID NO: 1943 - protein, 7 aa, synthetic construct
VDSHTTI

SEQ ID NO: 1944 - protein, 7 aa, synthetic construct
KGPLTDM

SEQ ID NO: 1945 - protein, 7 aa, synthetic construct
TTHQSPT

SEQ ID NO: 1946 - protein, 7 aa, synthetic construct
IDSSMAM

SEQ ID NO: 1947 - protein, 7 aa, synthetic construct
LGKQPTQ

SEQ ID NO: 1948 - protein, 7 aa, synthetic construct
SRGPNTL

SEQ ID NO: 1949 - protein, 7 aa, synthetic construct
AILSSPA

SEQ ID NO: 1950-1952 - skipped (3 entries)

(19) United States
(12) Patent Application Publication
(10) Pub. No.: US 2026/0028595 A1
(43) Pub. Date: Jan. 29, 2026

(54) SELECTED ADENO-ASSOCIATED VIRUS COMPOSITIONS HAVING PREFERRED BRAIN, SPINAL CORD, AND/OR HEART EXPRESSION LEVELS

(71) Applicant: CAPSIDA BIOTHERAPEUTICS, INC., Thousand Oaks, CA (US)

(72) Inventors: Brandon G. Wheeler, Thousand Oaks, CA (US); Xiaojing Shi, Thousand Oaks, CA (US); Nicholas S. Goeden, Thousand Oaks, CA (US); Nicholas C. Flytzanis, Thousand Oaks, CA (US); Troy E. Sandberg, Thousand Oaks, CA (US)

(21) Appl. No.: 18/862,827

(22) PCT Filed: May 16, 2023
(86) PCT No.: PCT/US2023/067059

§ 371 Date: Nov. 4, 2024

Related U.S. Application Data
(60) Provisional application No. 63/342,730, filed on May 17, 2022.

(51) Int. Cl. C12N 7/00; C07K 14/015

(52) U.S. Cl. CPC C12N 7/00; C07K 14/015; C12N 2750/14122

(57) ABSTRACT

Described herein are compositions and kits comprising recombinant adeno-associated viruses (rAAVs) with increased viral transduction enrichment in the brain, spinal cord, and/or heart. The rAAV compositions described herein encapsidate a transgene, such as a therapeutic nucleic acid. Gene therapy using the rAAVs is described. Also described are methods of treating diseases of the brain, spinal cord, and/or heart-related diseases and conditions.

### BACKGROUND

Recombinant adeno-associated viruses (rAAVs) are widely used as vectors for gene delivery in therapeutic applications because of their ability to transduce both dividing and non-dividing cells, their long-term persistence as episomal DNA in infected cells, and their low immunogenicity. These characteristics make them appealing for applications in therapeutic applications, such as gene therapy. However, there is a need to significantly improve the performance of existing AAV serotypes to selectively and efficiently express in distinct cell-types, upon systemic delivery to a subject. This need is especially acute when the AAV must be expressed in the brain, spinal cord, and/or the heart.

### SUMMARY OF THE INVENTION

Disclosed herein are rAAVs with engineered transduction enrichment into the capsid structure through iterative rounds of selection in non-human primates (NHPs), yielding variants having increased transduction enrichment when measured in the brain, spinal cord, and/or heart. In particular, these variants yield increased tissue-specific transduction enrichment when compared to rAAVs with a native AAV sequence. Furthermore, during the development of the rAAVs of the invention, variants with increased transduction enrichment in different or non-target tissues were discovered. These variants inform the further development of rAAVs that specifically target the brain, spinal cord, and/or heart as they reveal variants and motifs to avoid in order to prevent non-tissue specific transduction enrichment.

The present invention provides rAAVs with widespread transduction to the brain, spinal cord, and/or heart. In certain aspects, the present invention provides rAAVs with widespread transduction to the brain, spinal cord, and/or heart.

The present invention provides, in an aspect, a peptide insertion sequence comprising or consisting of an amino-acid sequence set forth in any one of Table 1, FIG. 1 and/or Formulas I-III.

Another aspect of the invention is a modified capsid protein wherein the AAV capsid protein, with a peptide insertion comprising or consisting of an amino-acid sequence set forth in any one of Table 1, FIG. 1 and/or Formulas I-III, is characterized by enriched brain, spinal cord, and/or heart transduction in a subject. Another aspect of the invention is a modified capsid protein wherein the AAV capsid protein, with a peptide insertion comprising or consisting of an amino-acid sequence set forth in any one of Table 1, FIG. 1 and/or Formulas I-III, is characterized by enriched brain, spinal cord, and/or heart transduction in a subject.

The present disclosure moreover includes pharmaceutical compositions comprising rAAVs with a peptide insertion comprising or consisting of an amino-acid sequence set forth in any one of Table 1, FIG. 1 and/or Formulas I-III, and a pharmaceutically acceptable excipient.

Aspects disclosed herein provide methods of treating a disease or condition in a subject comprising administering a therapeutically effective amount of a pharmaceutical formulation comprising the AAV capsid protein or the AAV capsid of the present disclosure. In some embodiments, the disease or the condition is a disease or a condition of the brain, spinal cord, and/or heart of the subject. Relatedly, the invention includes use of the rAAVs in the manufacture of a medicament for treating or preventing the disease or medical condition.

Other aspects of the invention will be apparent from the detailed description and claims that follow.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides AAV capsid protein insertion and substitution amino acid sequences which were found in the non-human primate brain, spinal cord, and/or heart samples after two rounds of evaluating engineered AAV libraries. At the outset, 39,000 separate insertion sequences were evaluated, with the listed 975 inserts in FIG. 1 as showing the most tissue-specific enrichment. The sequences of the 7mer amino acid sequence insertions as described herein, and which were inserted after residue #587 of SEQ ID NO: 1 (the native AAV2 sequence), including the surrounding 4 amino acids at the insertion site are provided as SEQ ID NOS: 2-975. The corresponding 7mer sequences are also provided in FIG. 1 as SEQ ID NOS: 976-1949. The FIGURE shows the average enrichment of AAVs with the shown inserts in different types of tissue, including tissue of the brain, spinal cord pool (SCP), heart, and/or liver.

### DETAILED DESCRIPTION OF THE DISCLOSURE

In one aspect the disclosure provides rAAVs with high expression levels in the brain, spinal cord, and/or heart. In one aspect the disclosure provides rAAVs with high expression levels in the brain. In one aspect the disclosure provides rAAVs with high expression levels in the spinal cord. In one aspect the disclosure provides rAAVs with high expression levels in the heart. In one aspect the disclosure provides rAAVs with low expression levels in the liver. In one aspect, the disclosure provides rAAVs that are developed to specifically avoid certain peptide sequences and/or motifs to avoid non-target tissue expression.

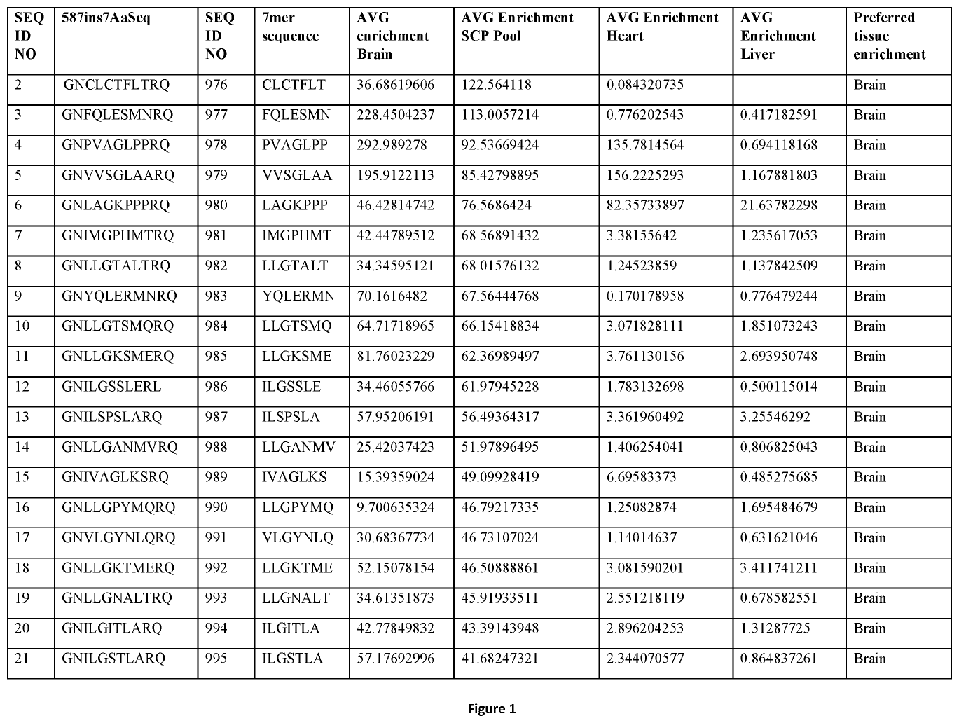

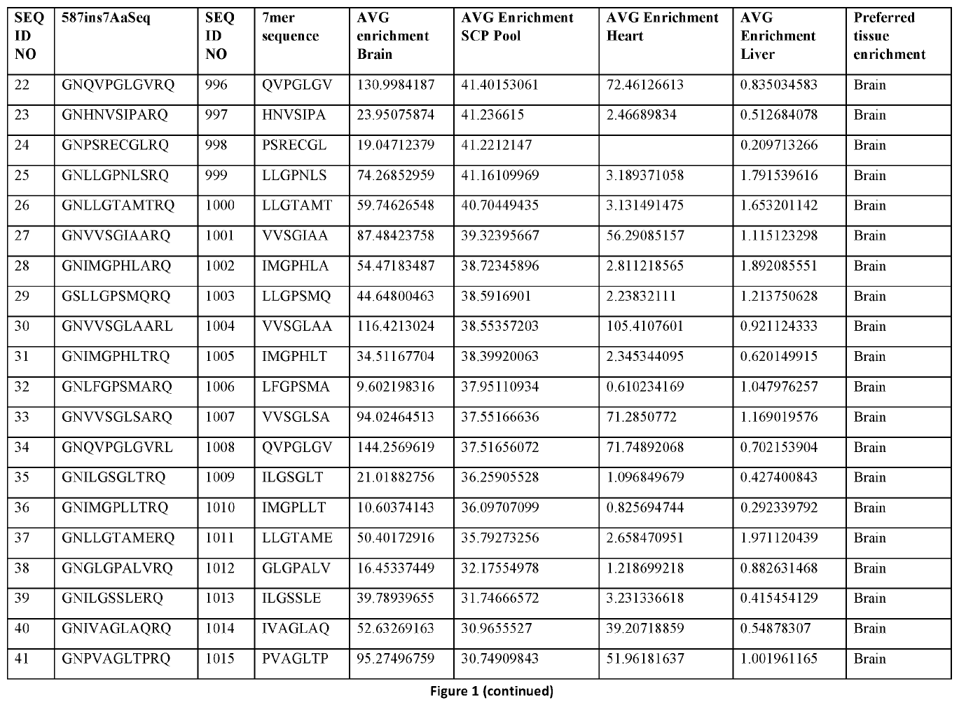

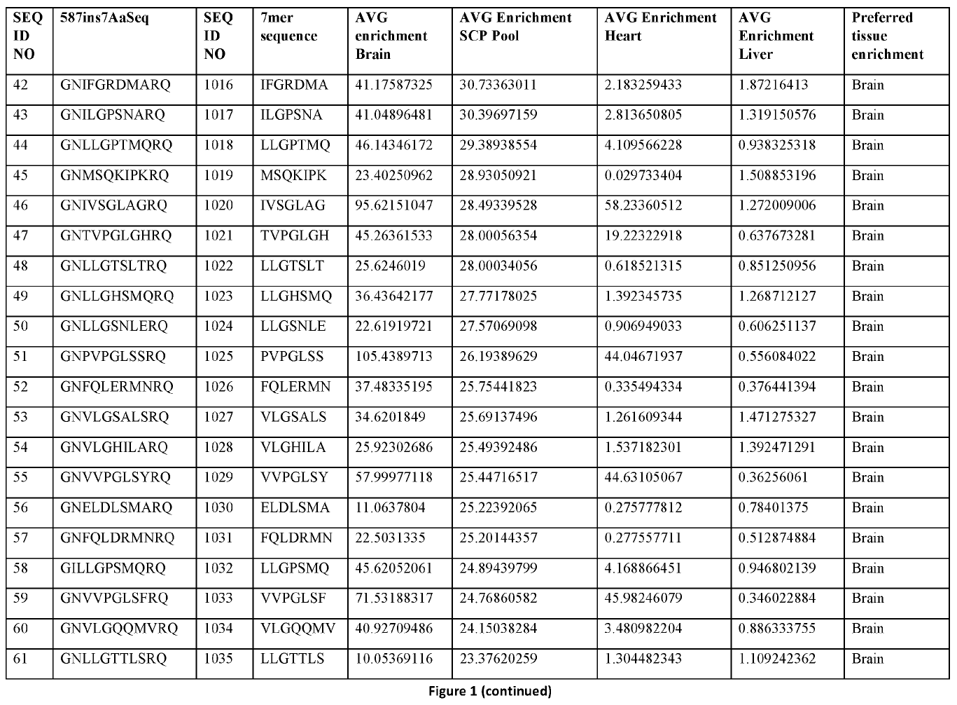

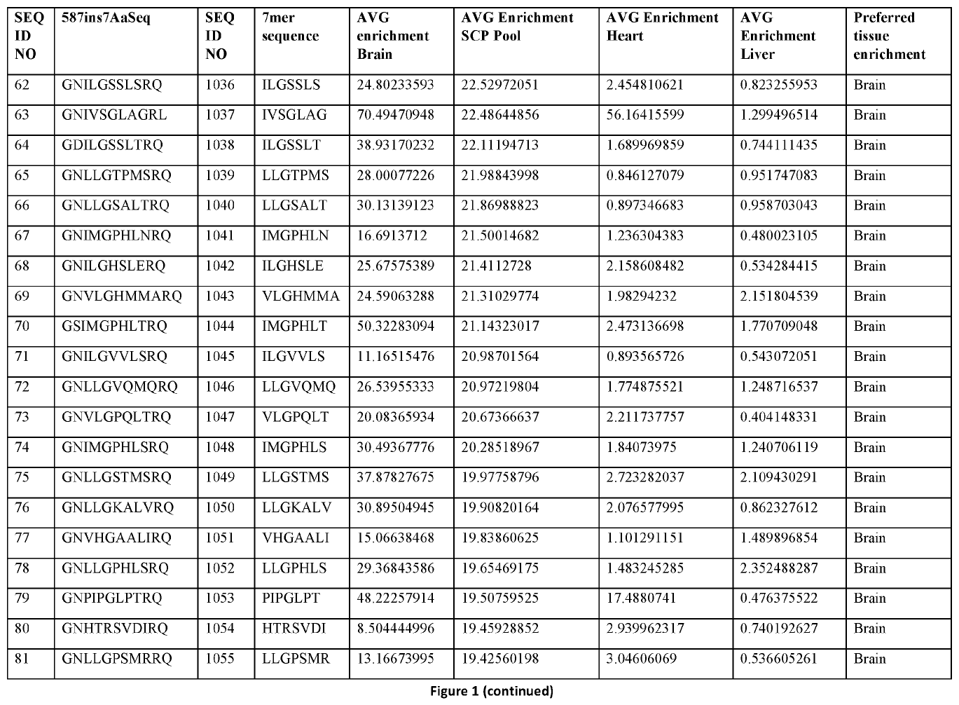

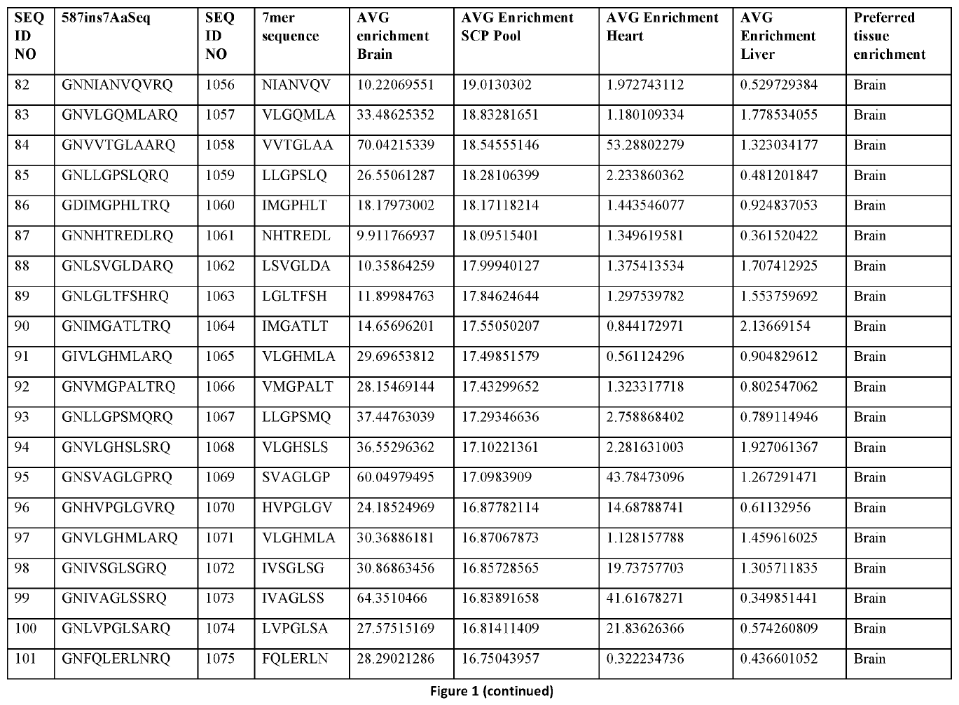

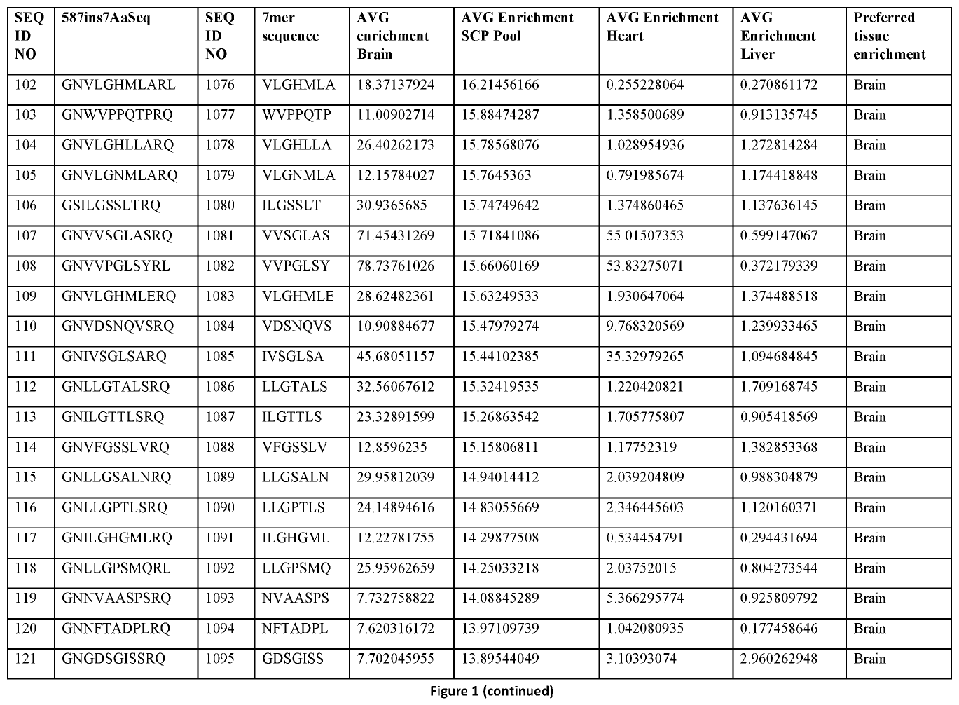

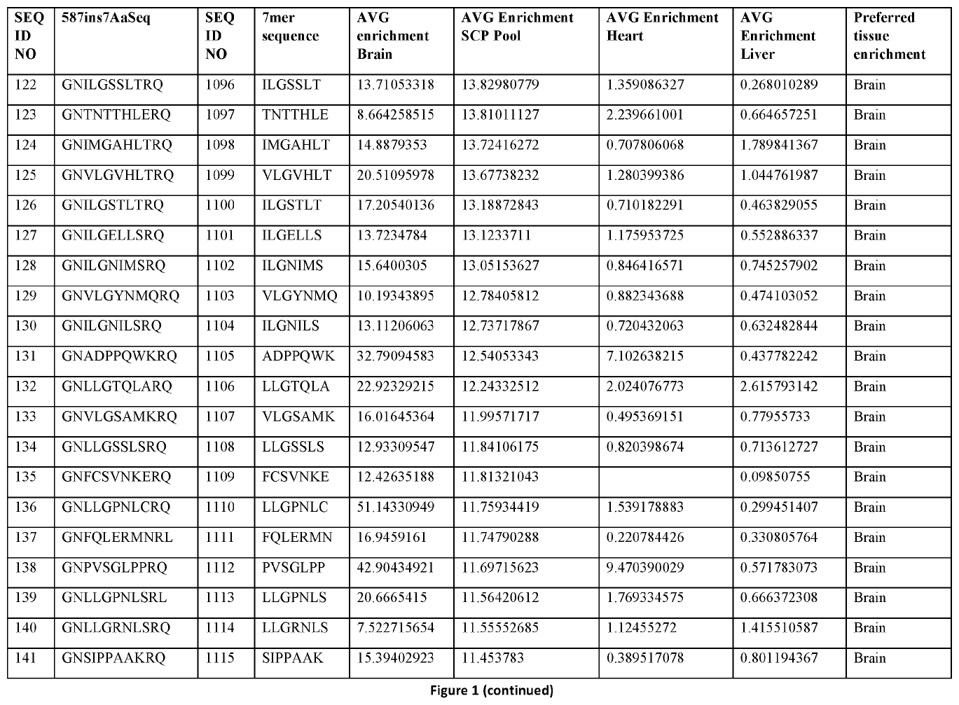

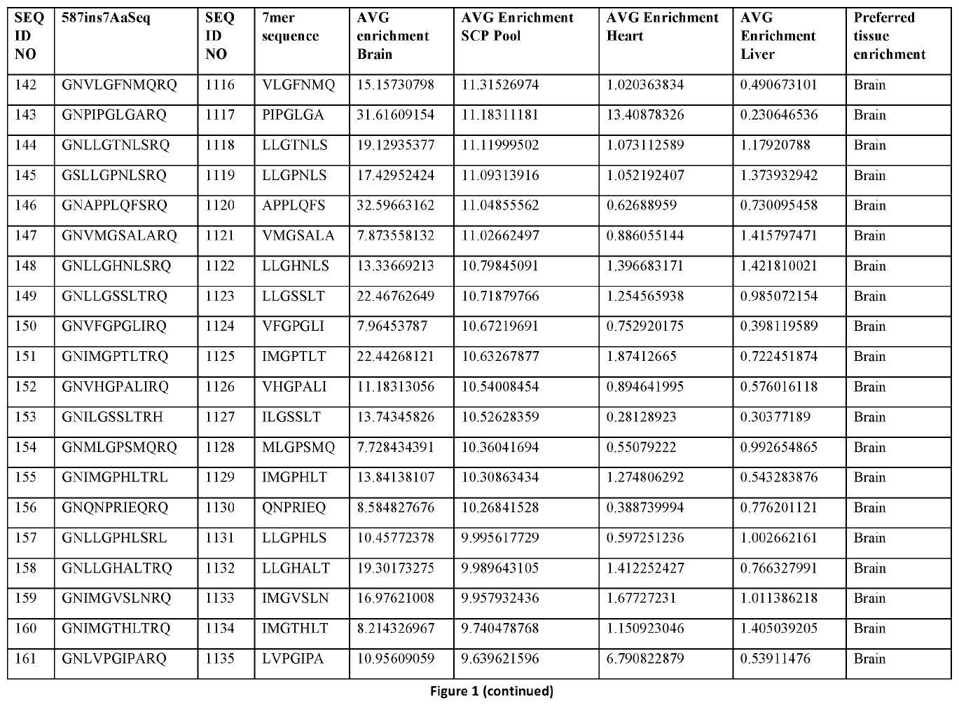

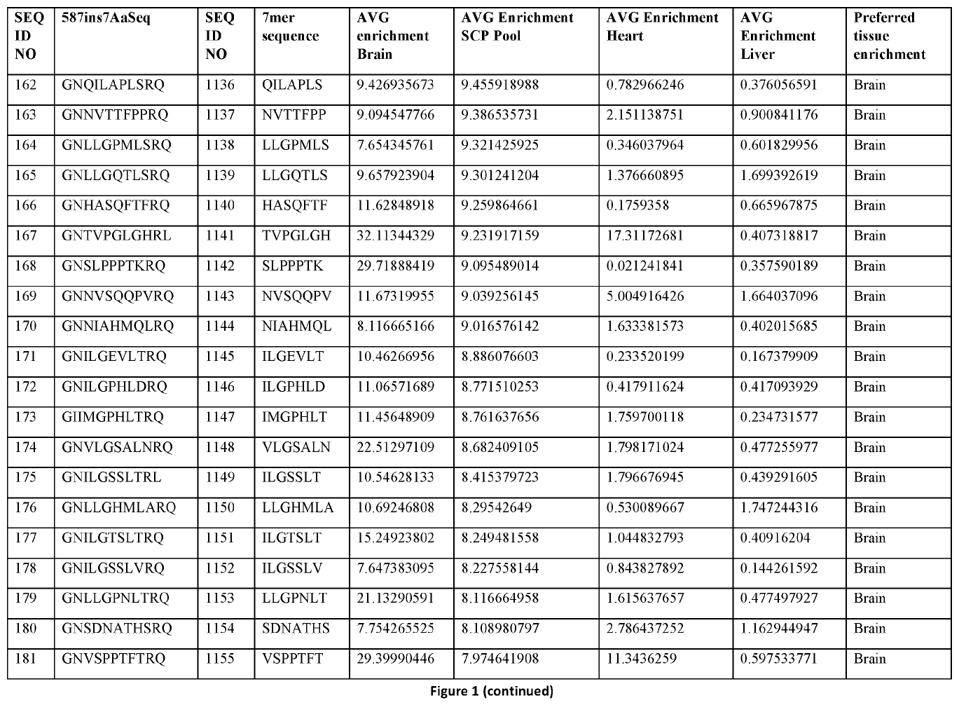

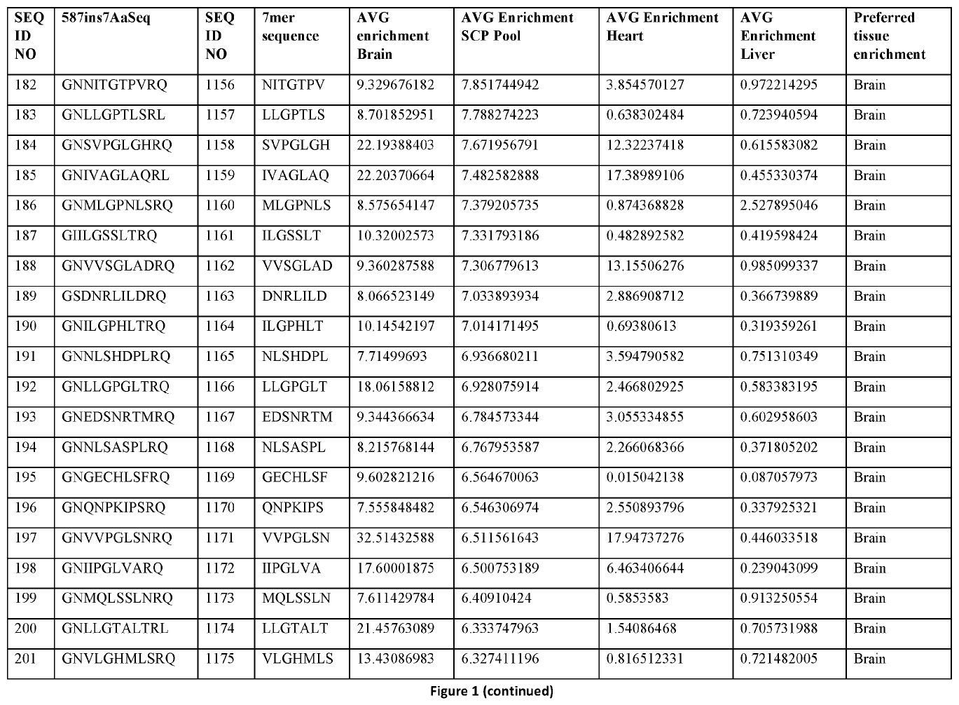

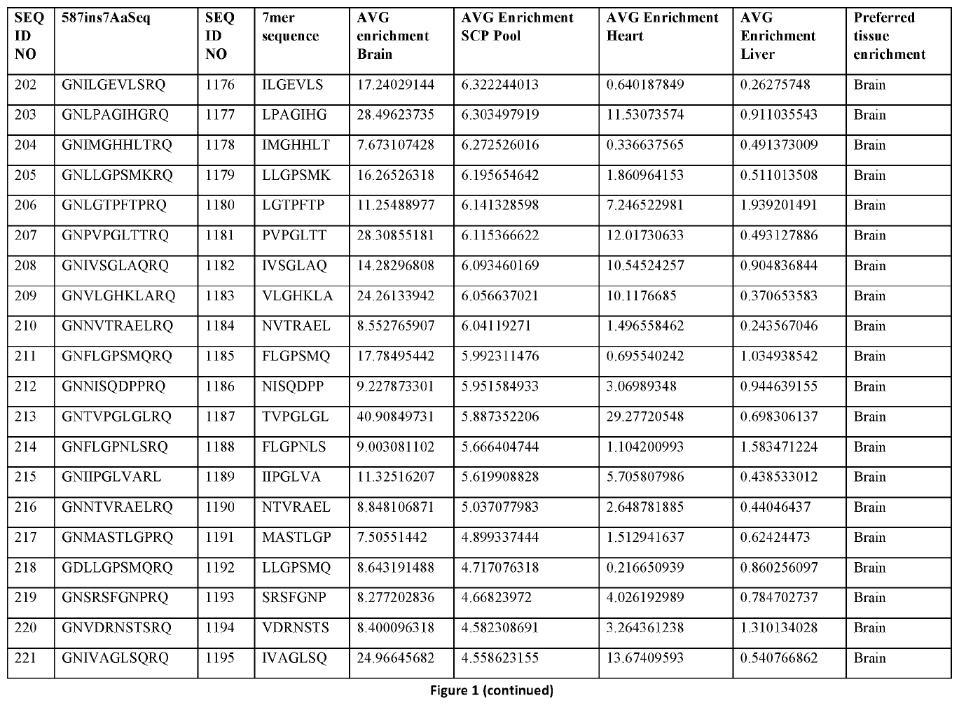

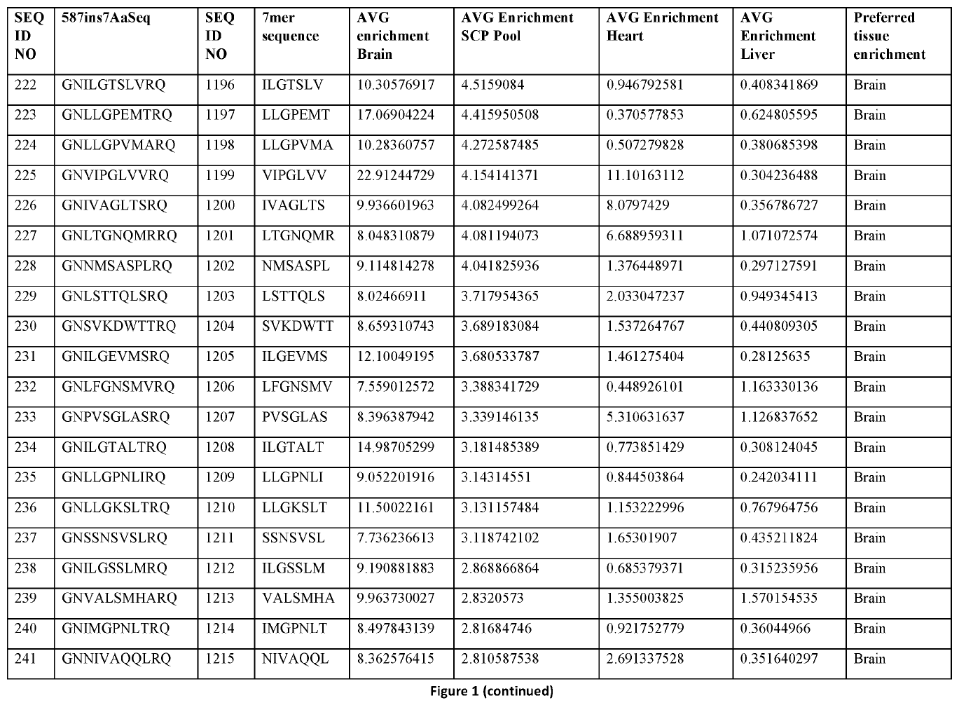

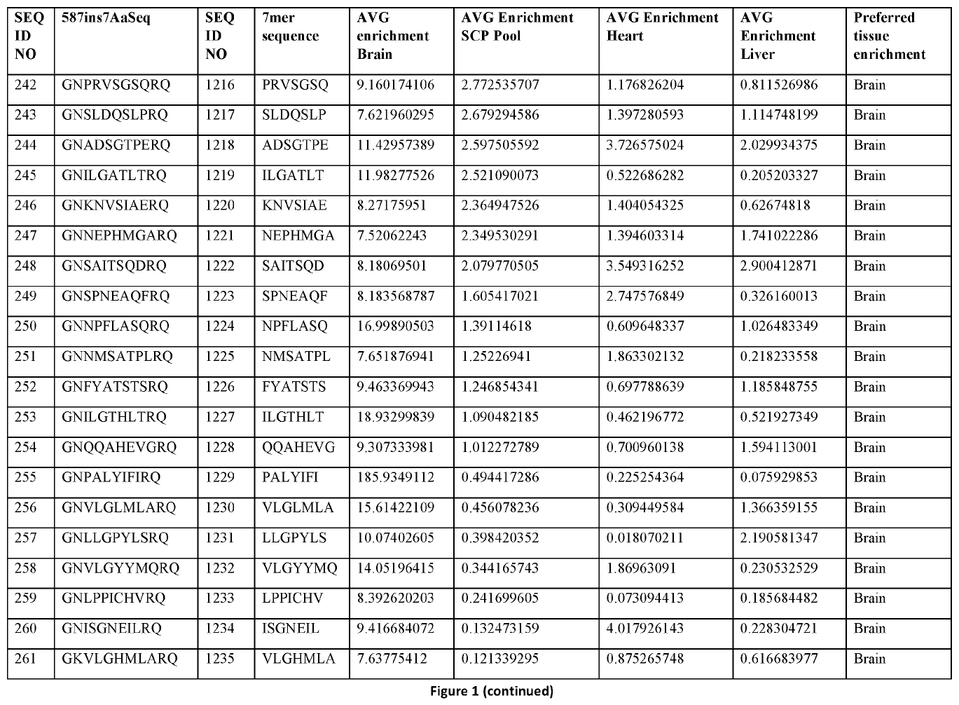

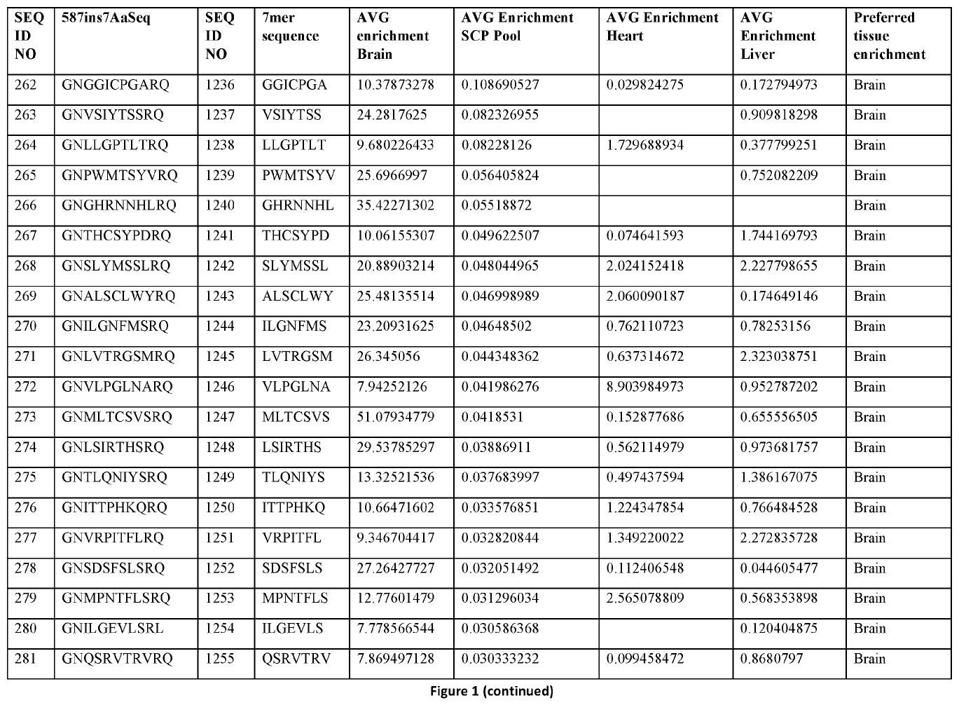

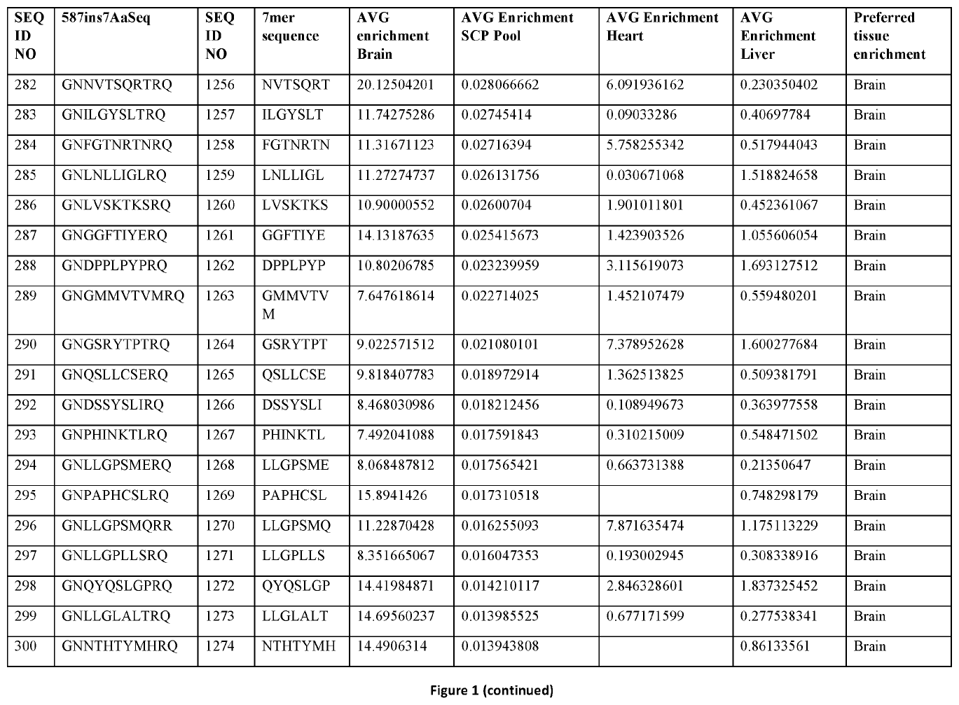

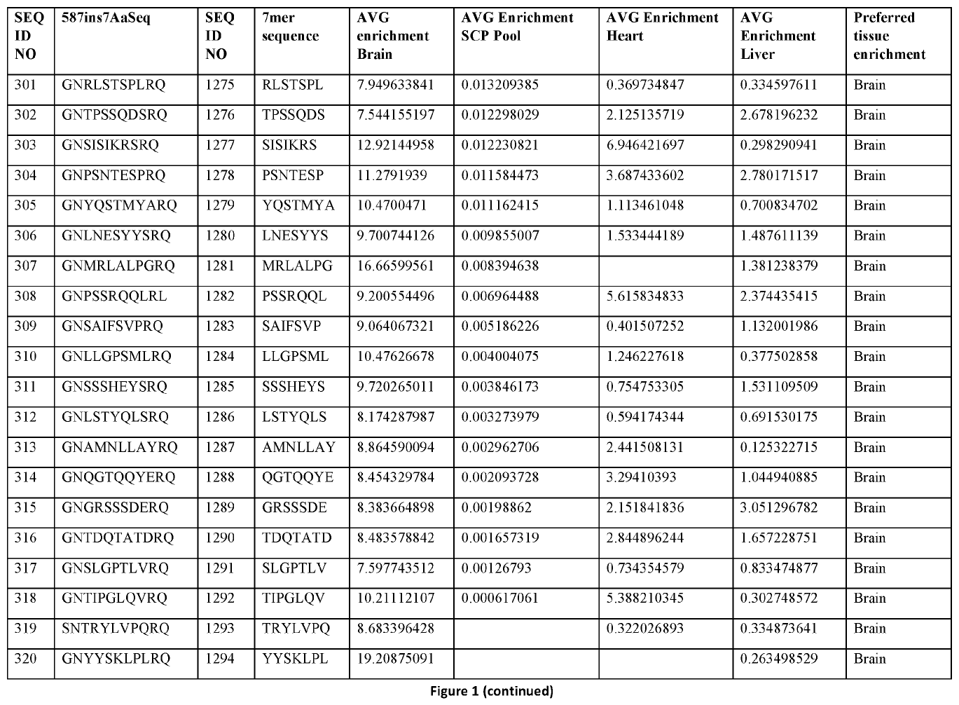

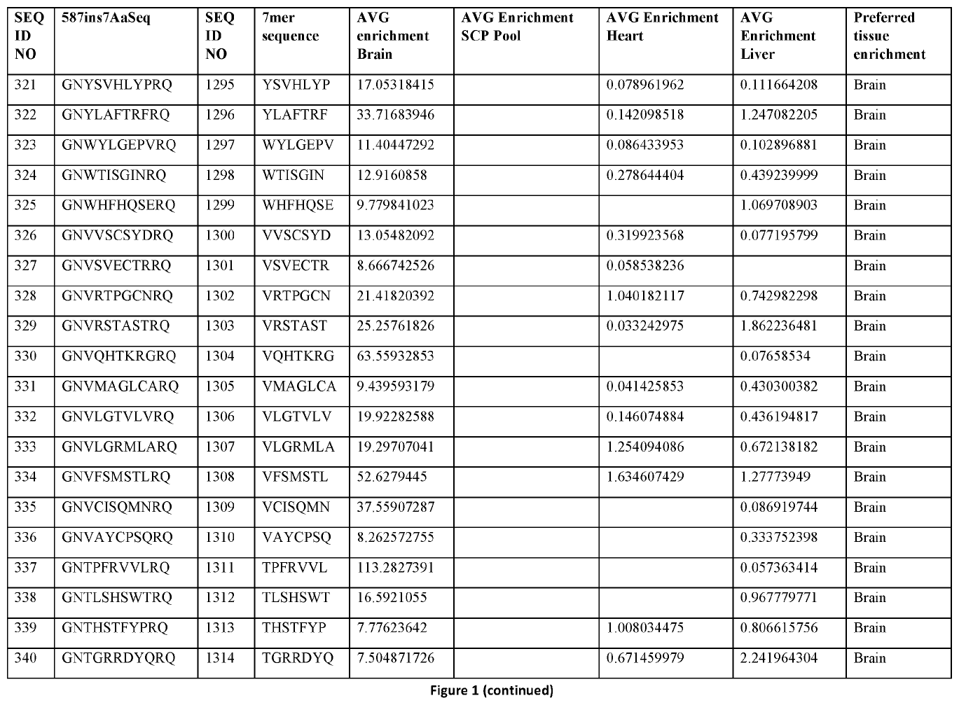

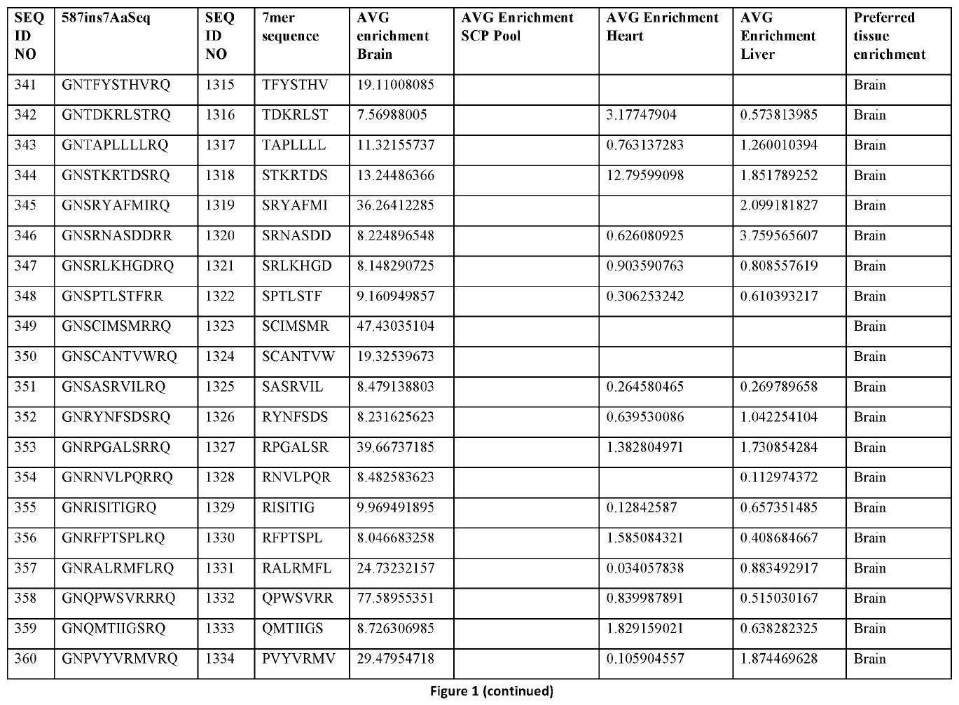

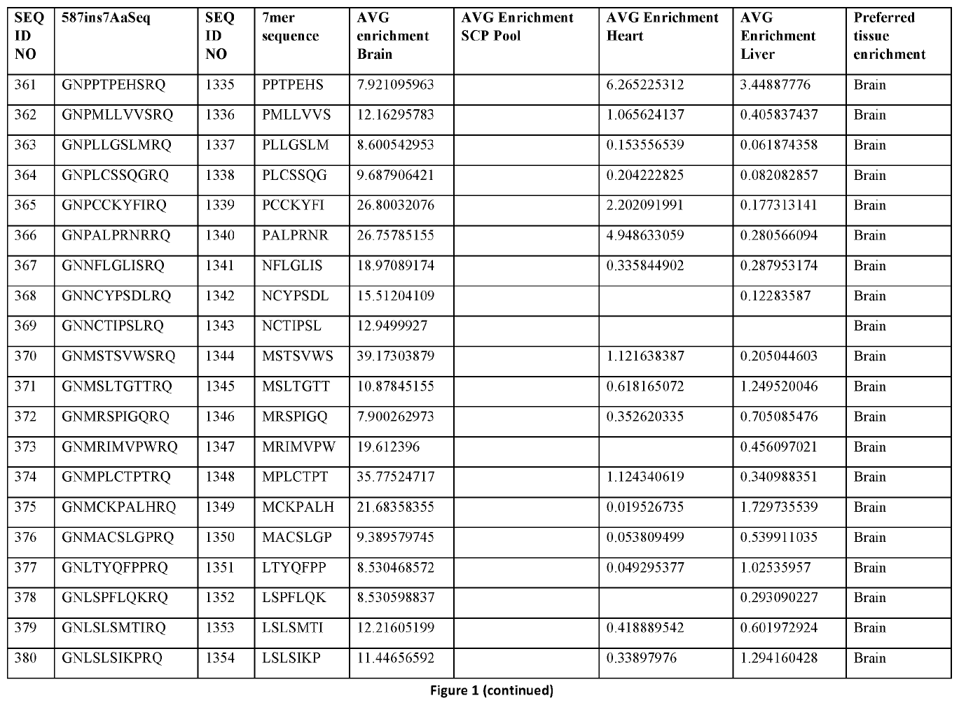

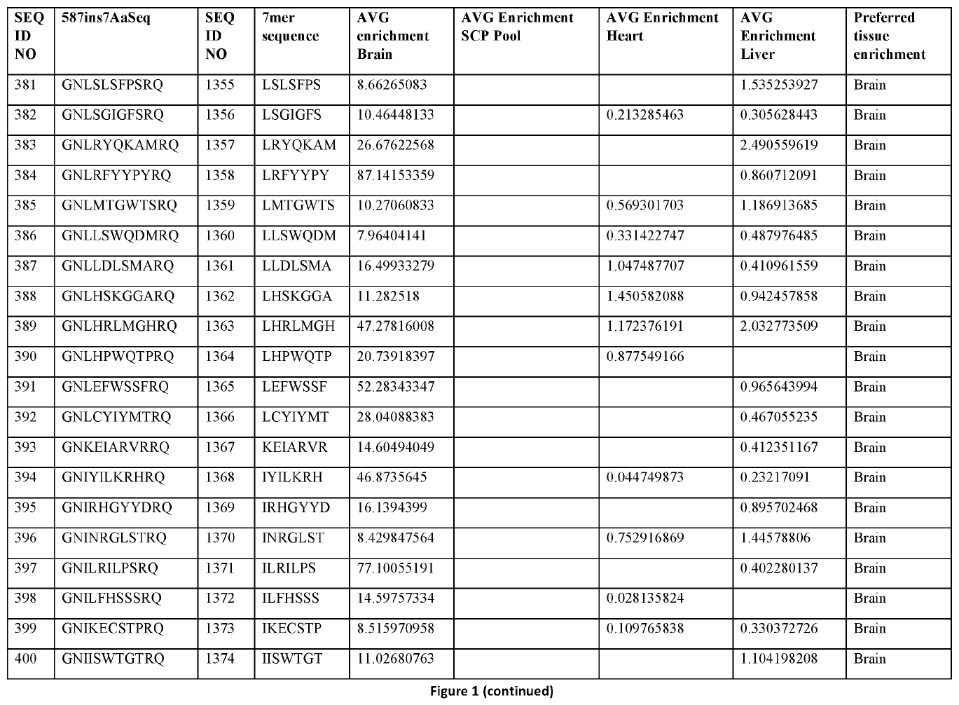

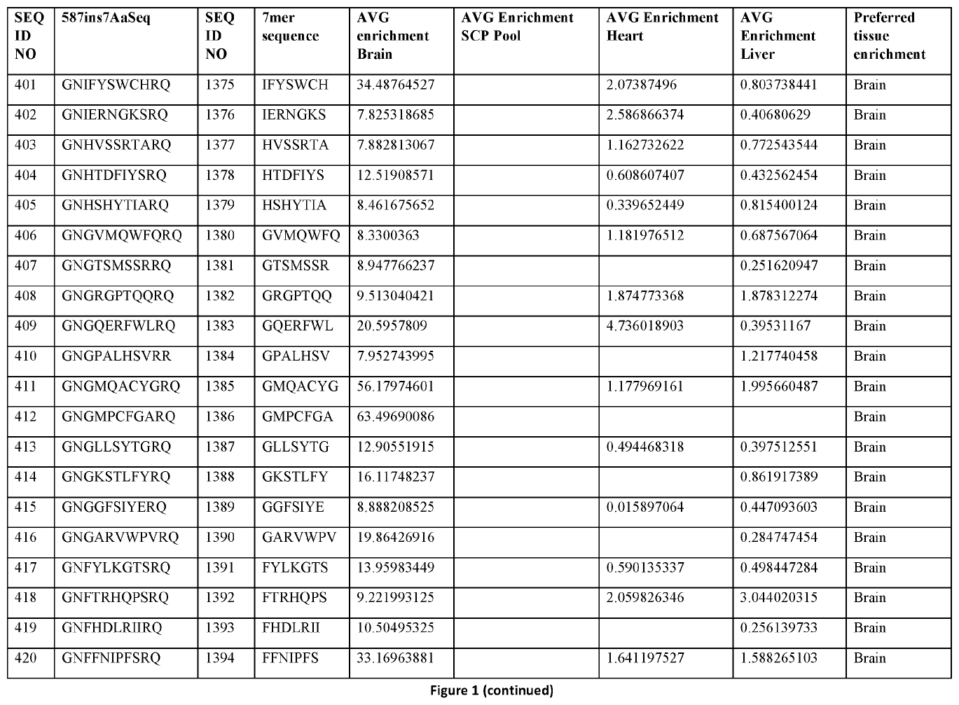

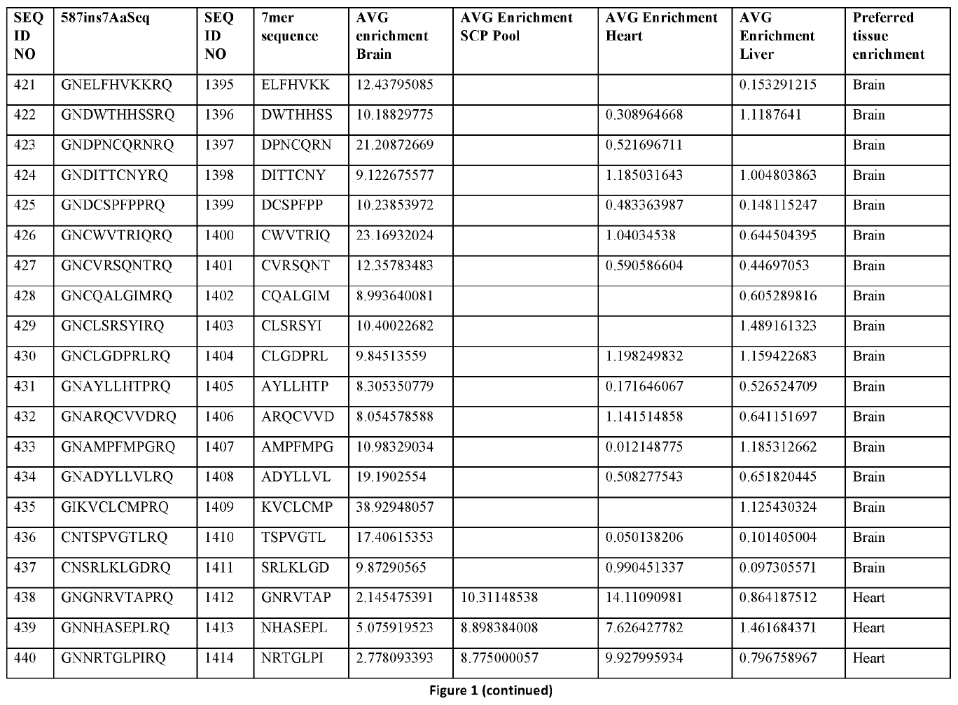

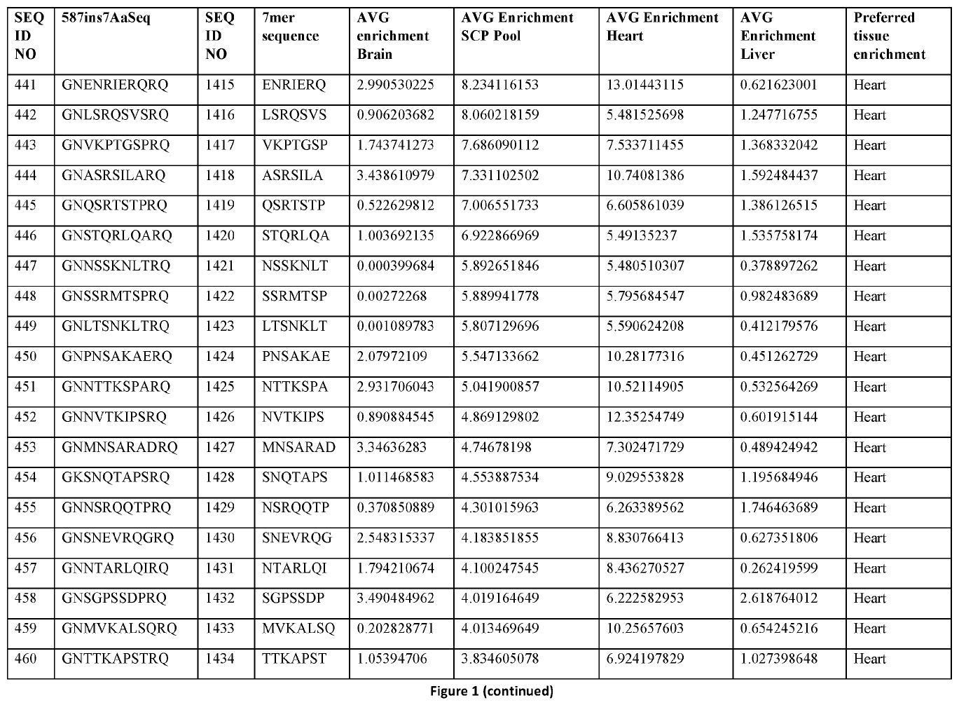

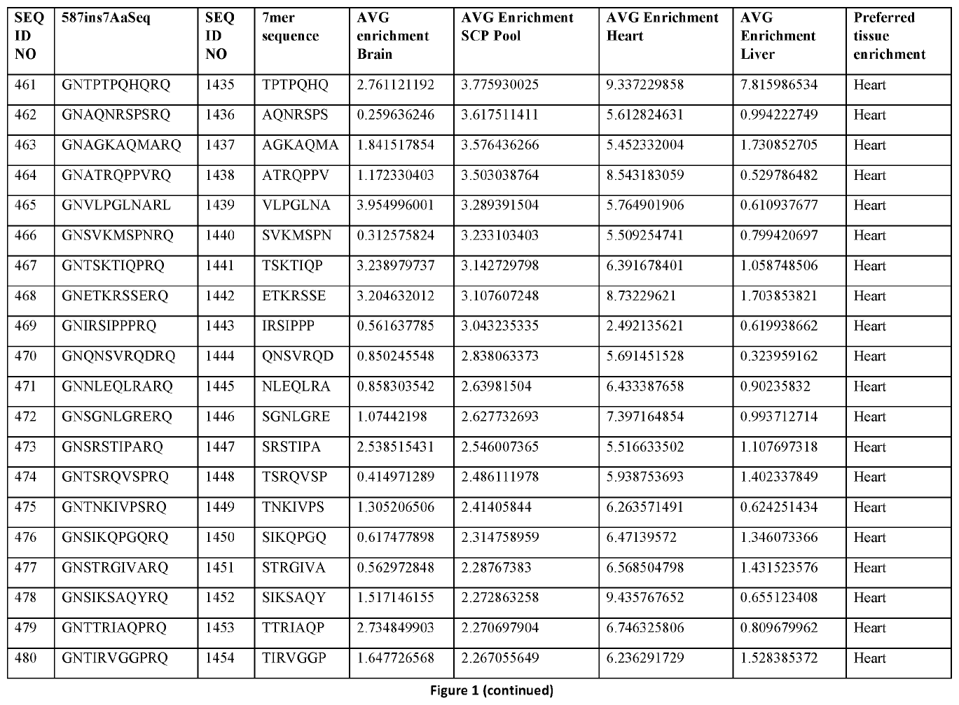

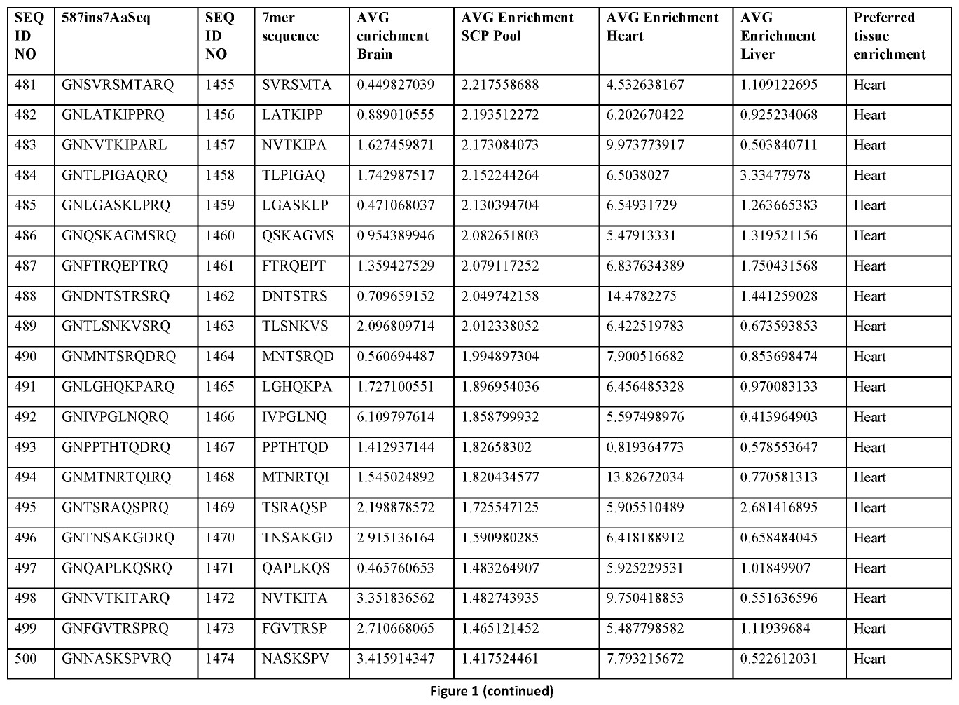

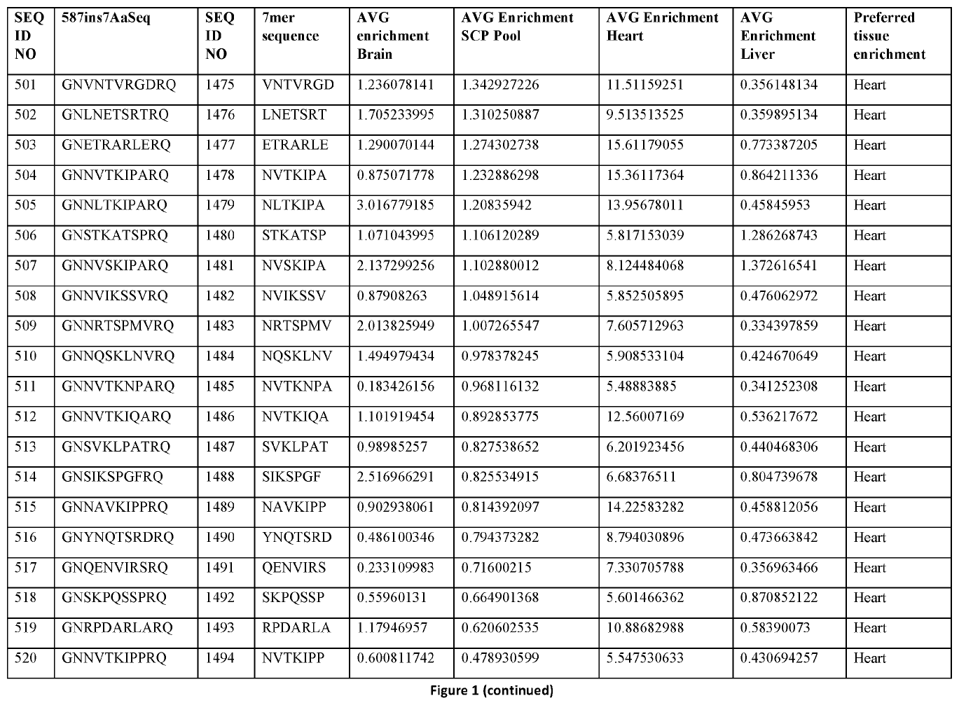

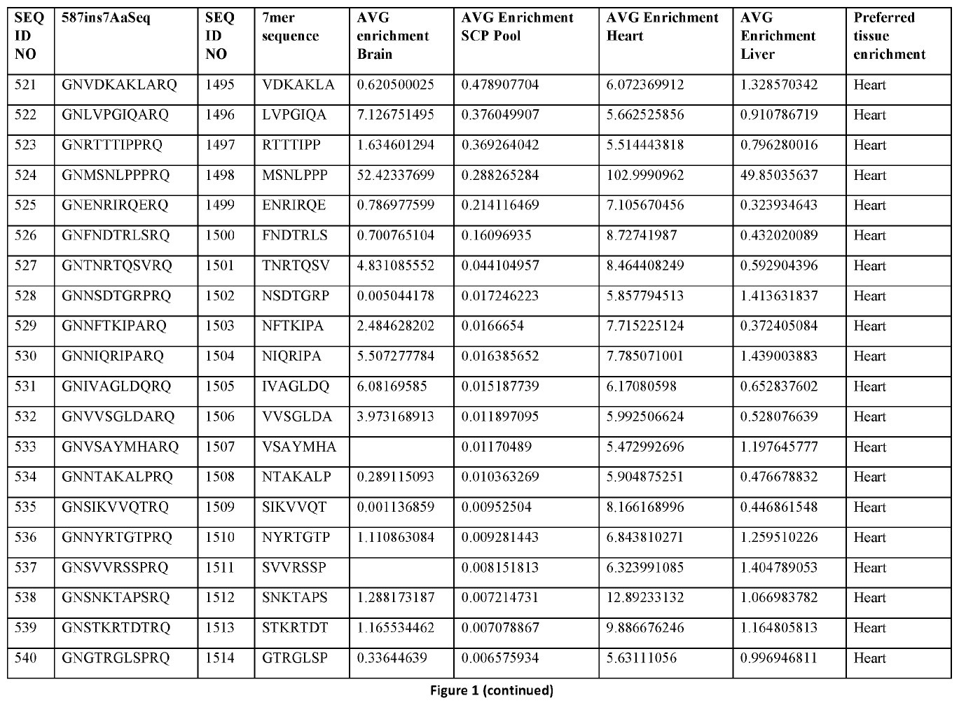

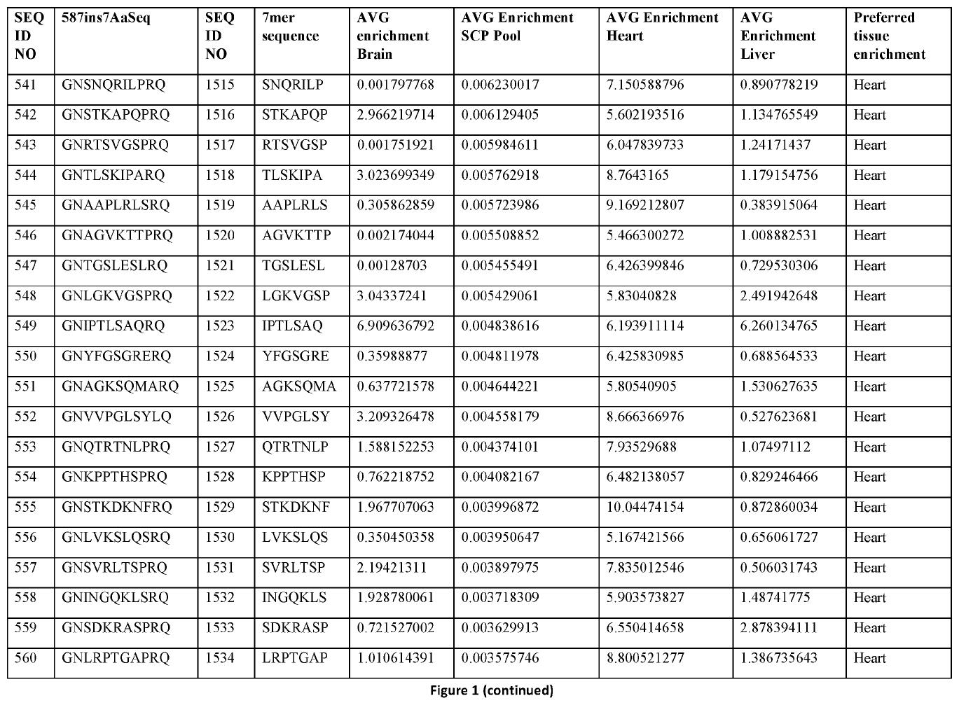

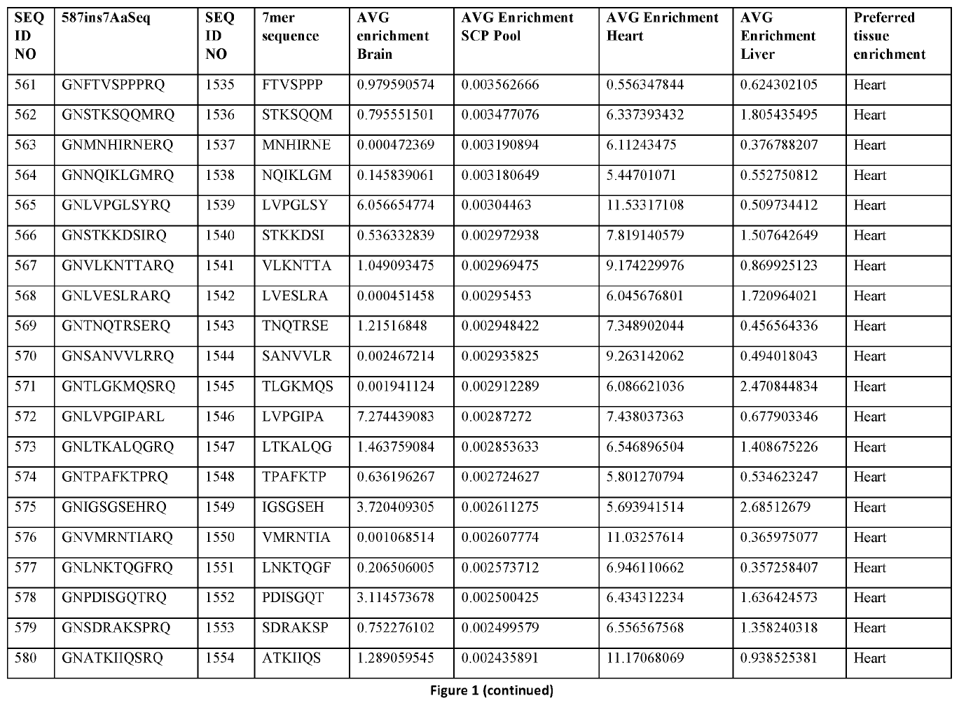

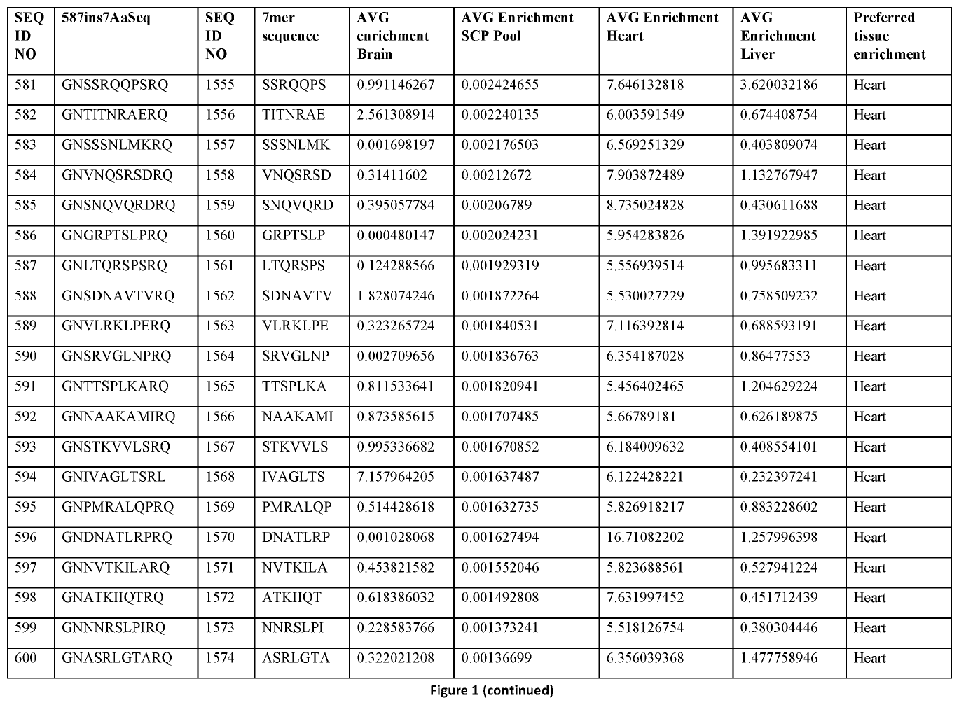

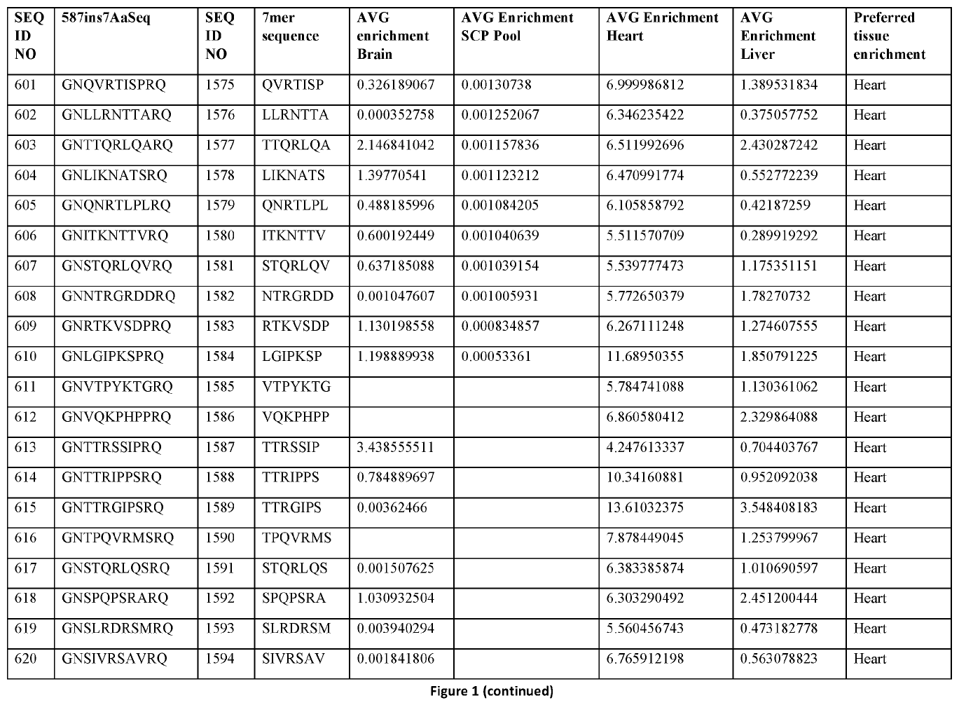

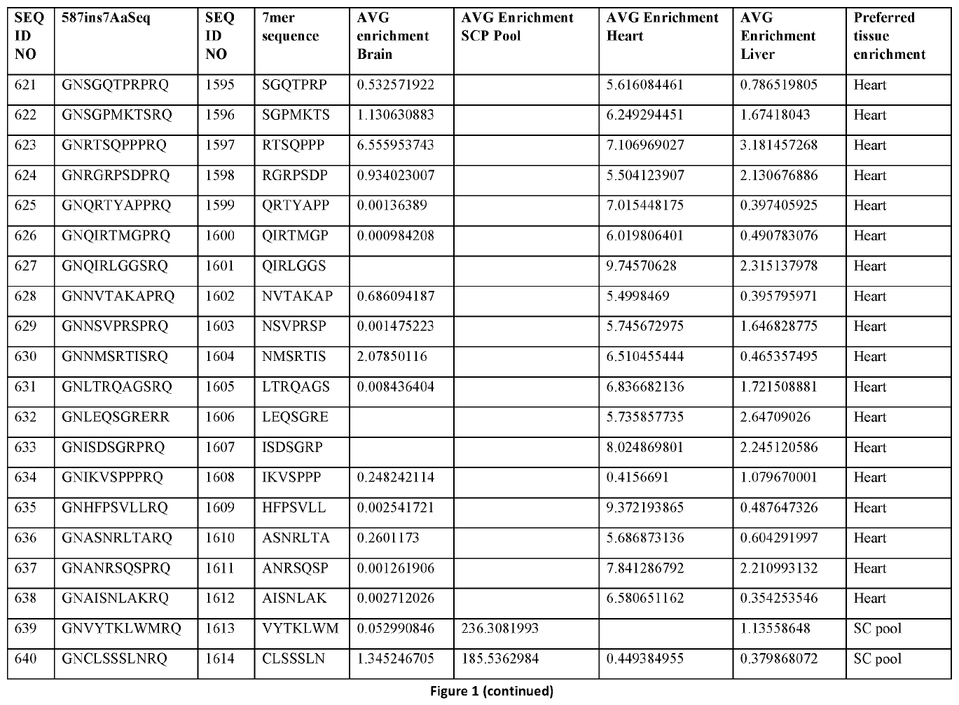

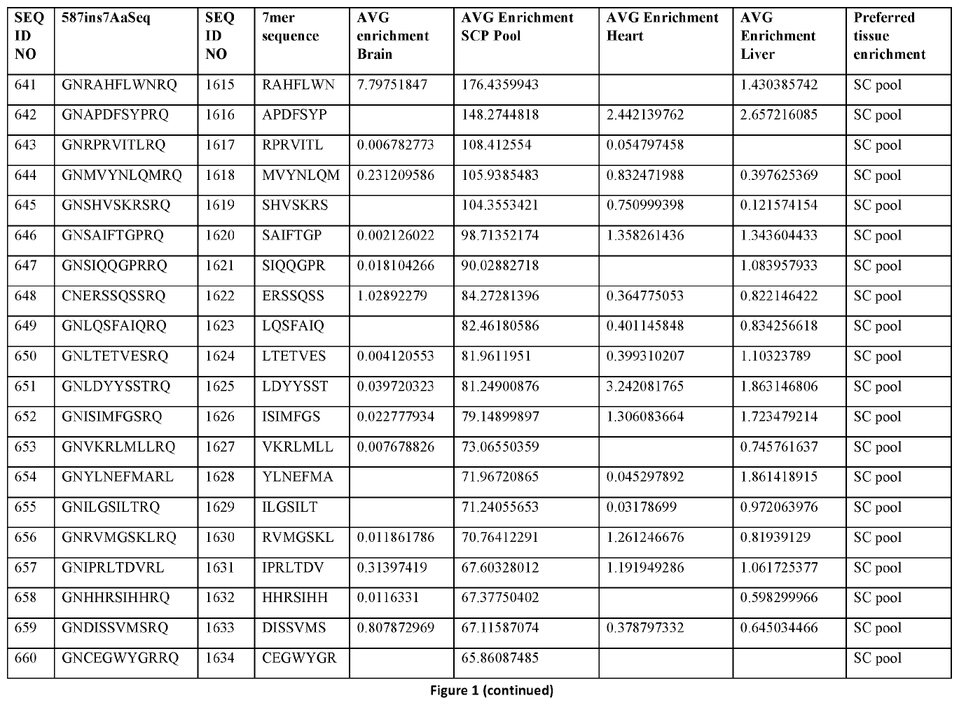

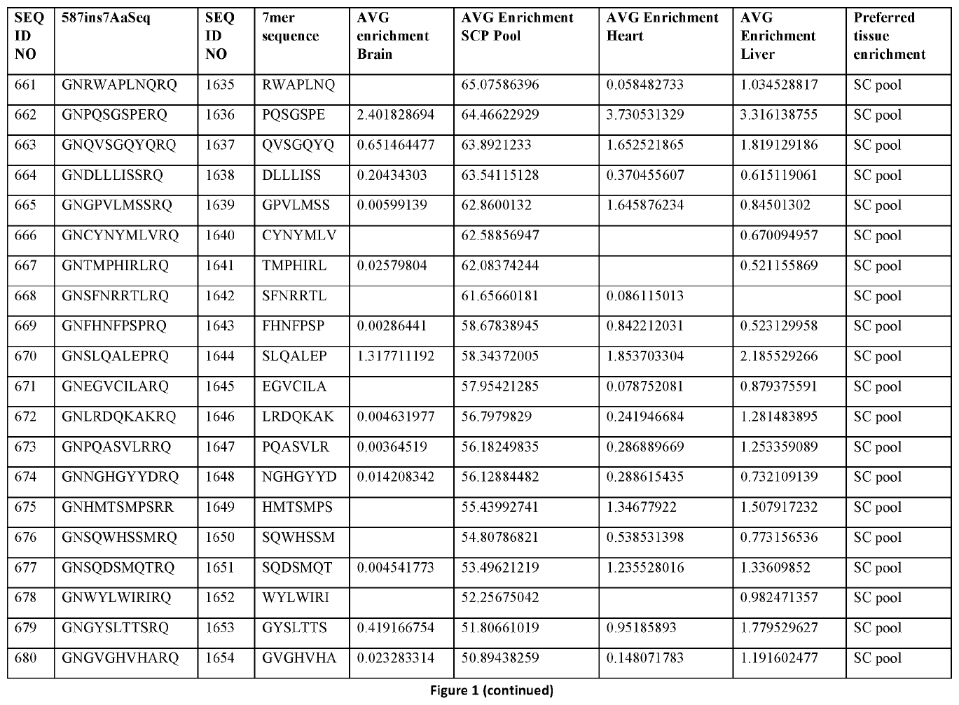

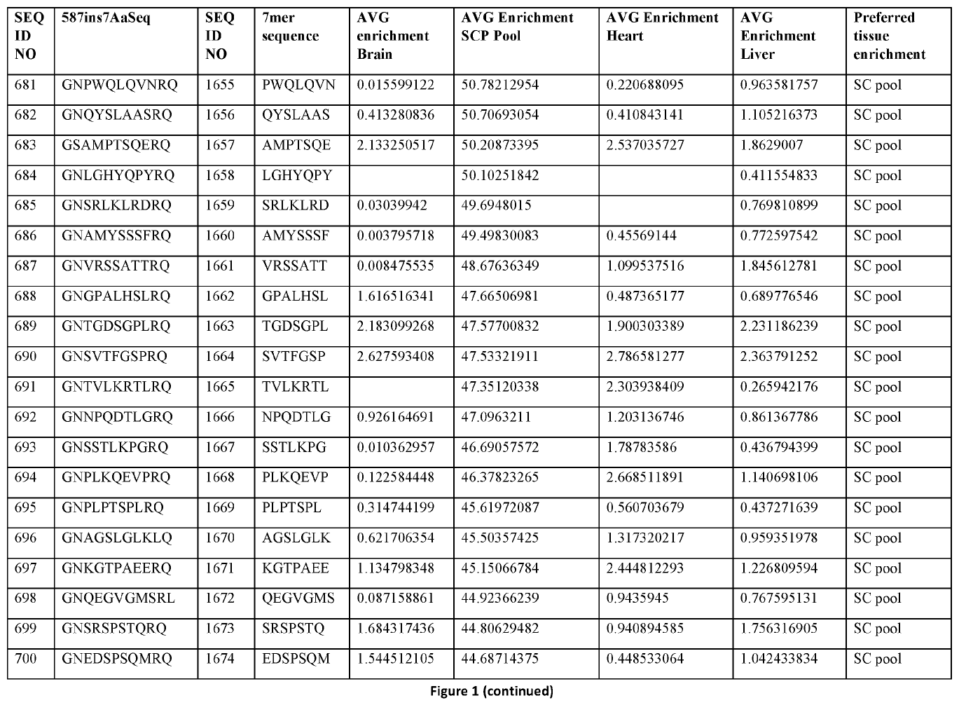

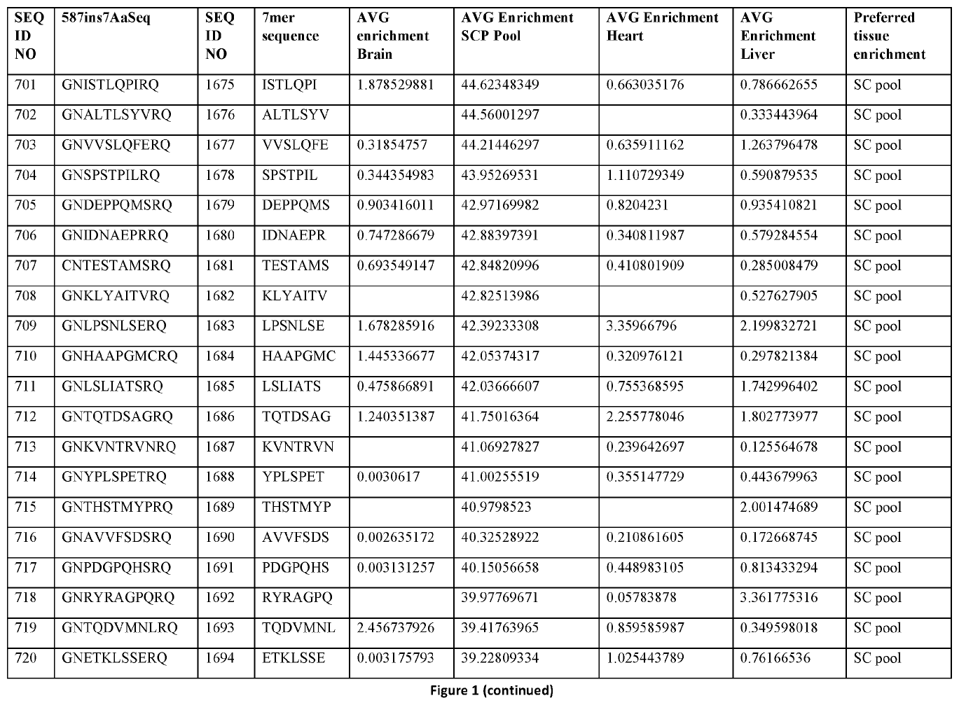

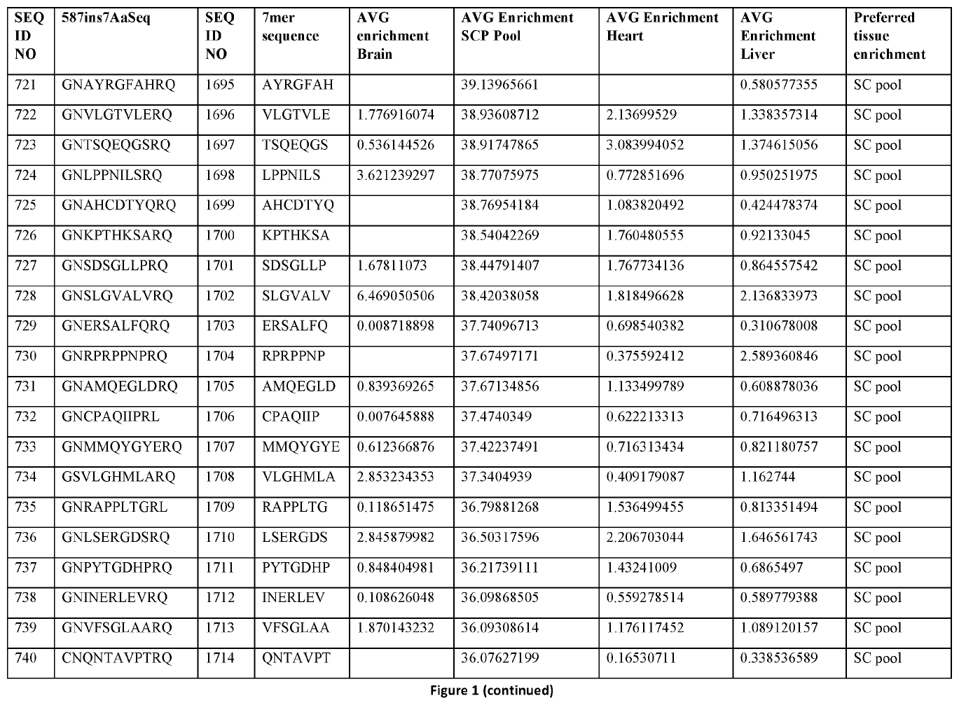

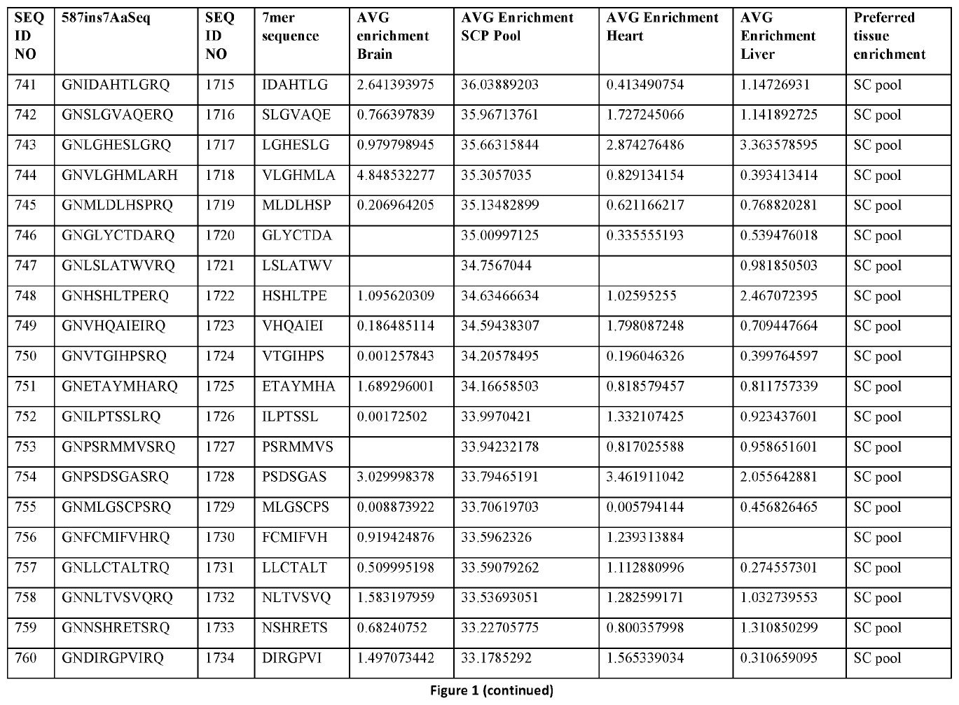

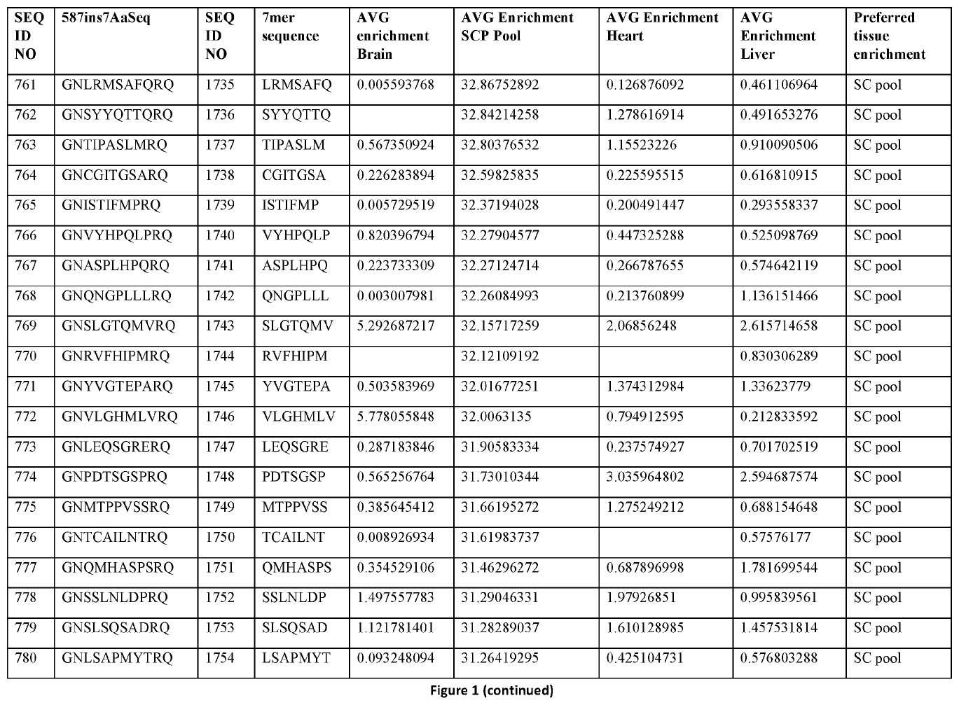

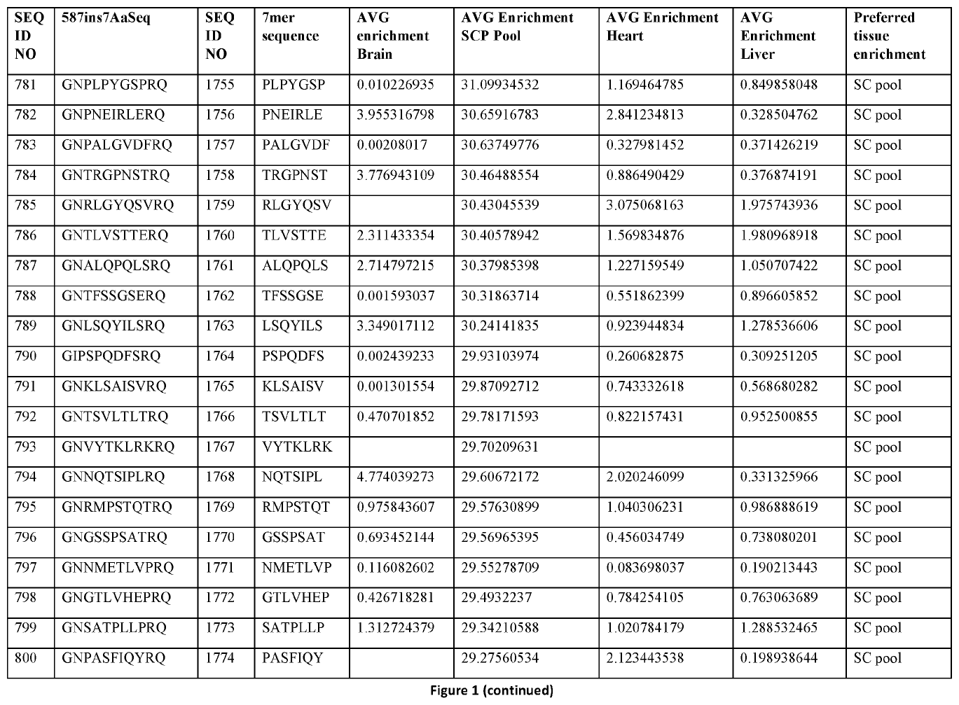

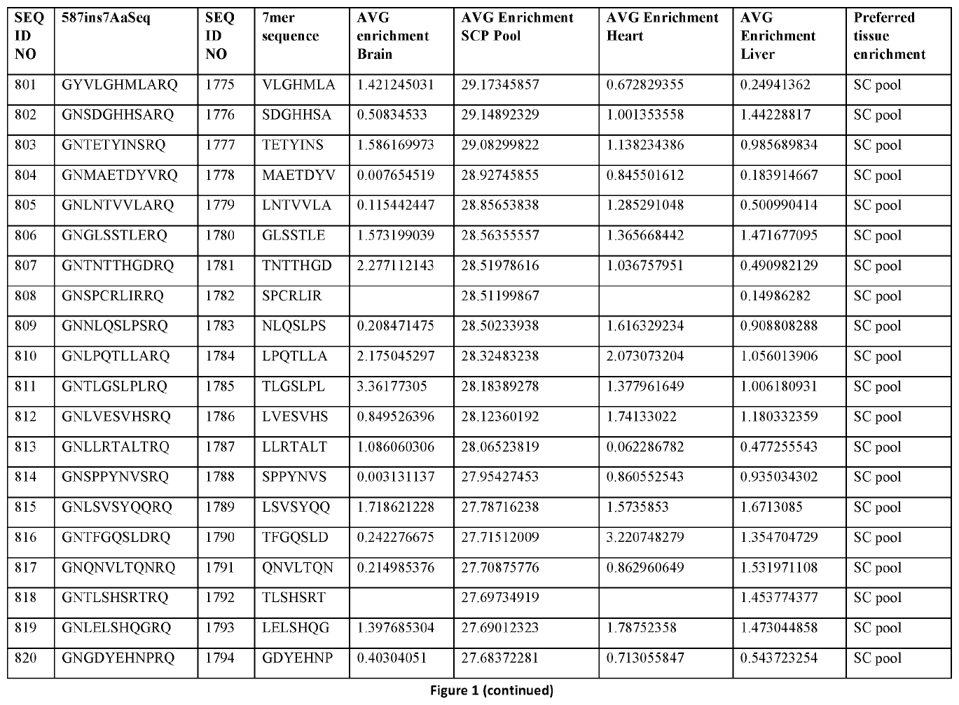

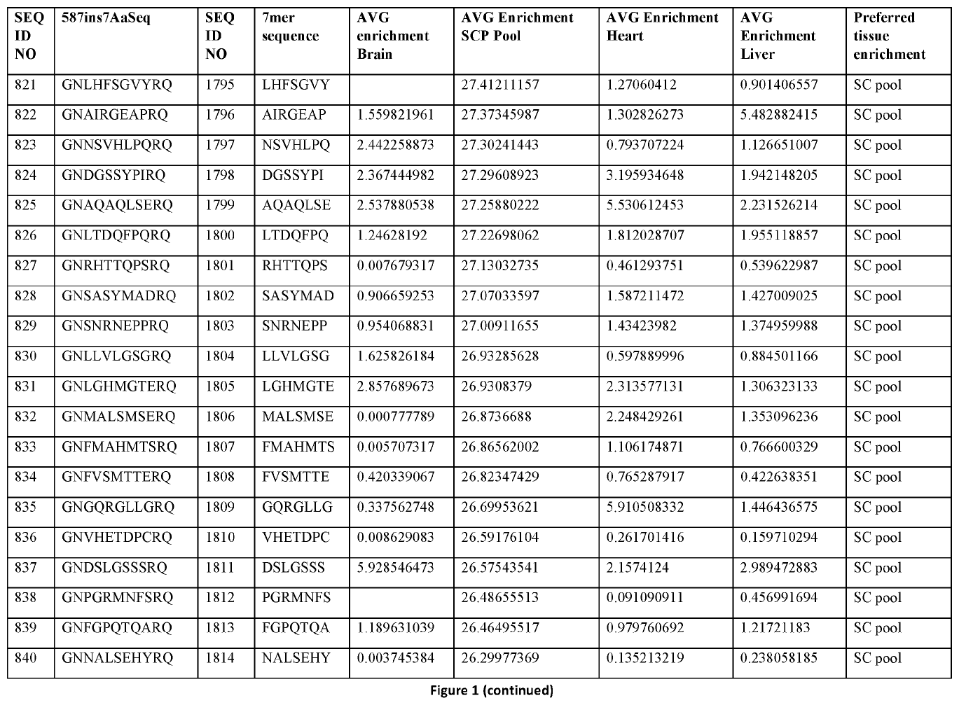

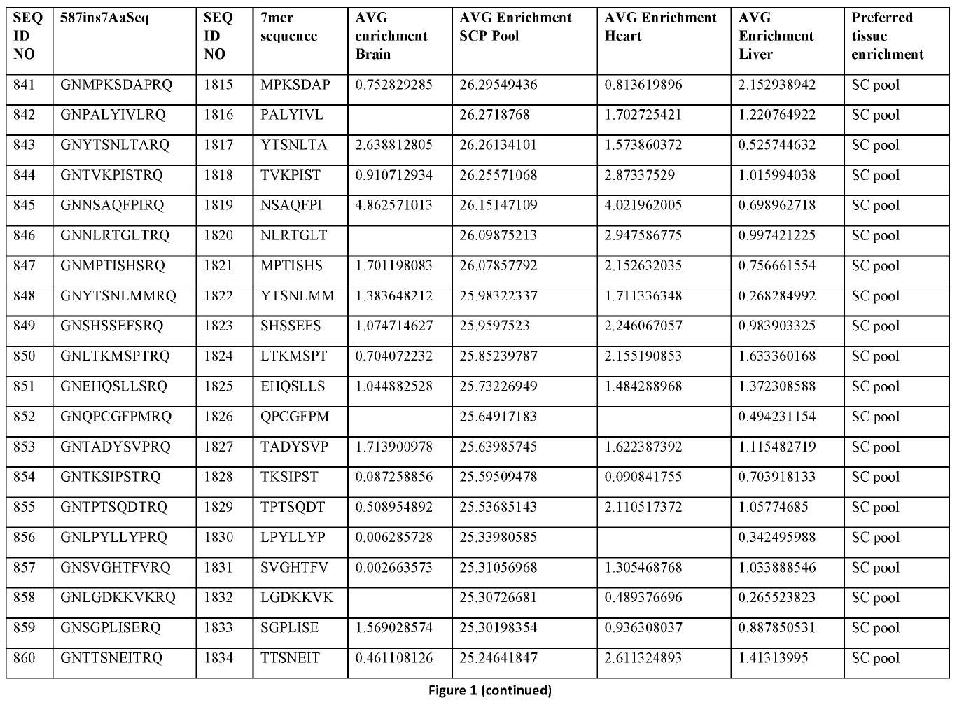

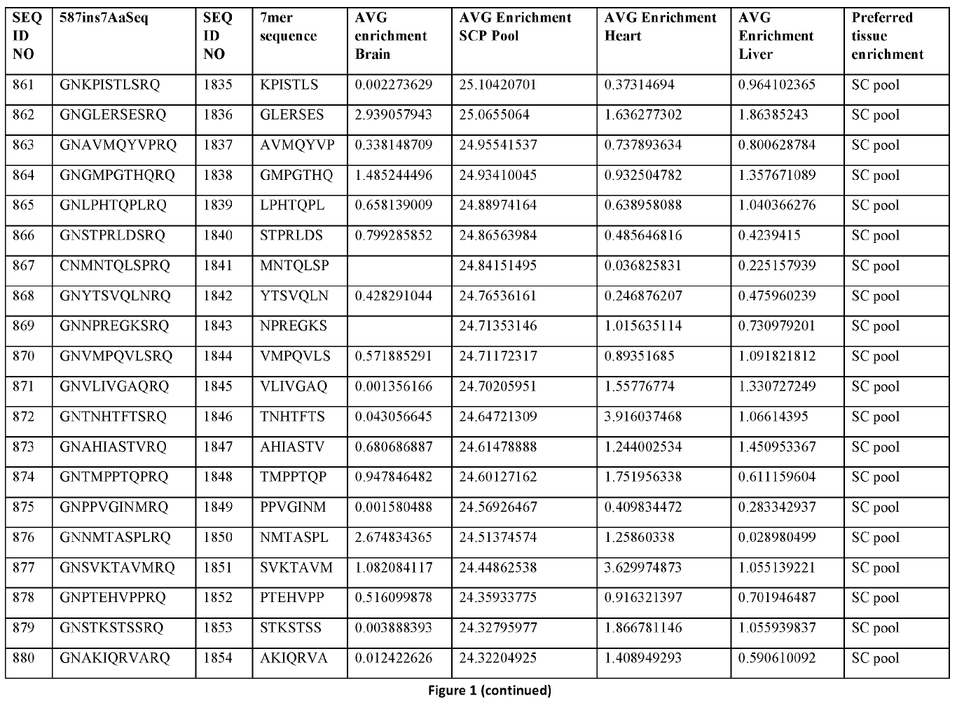

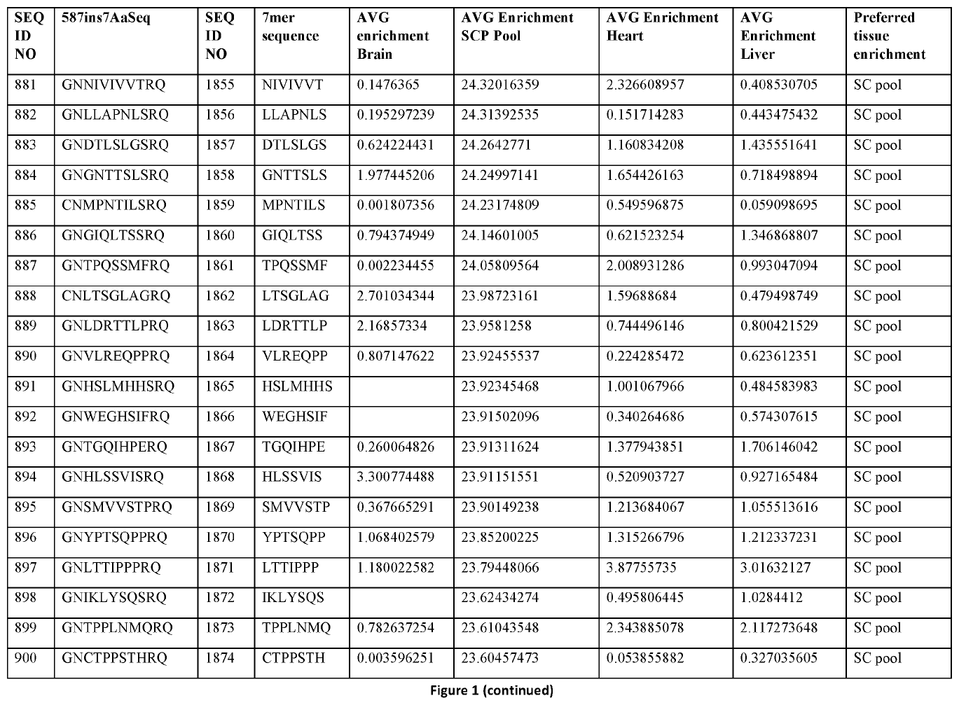

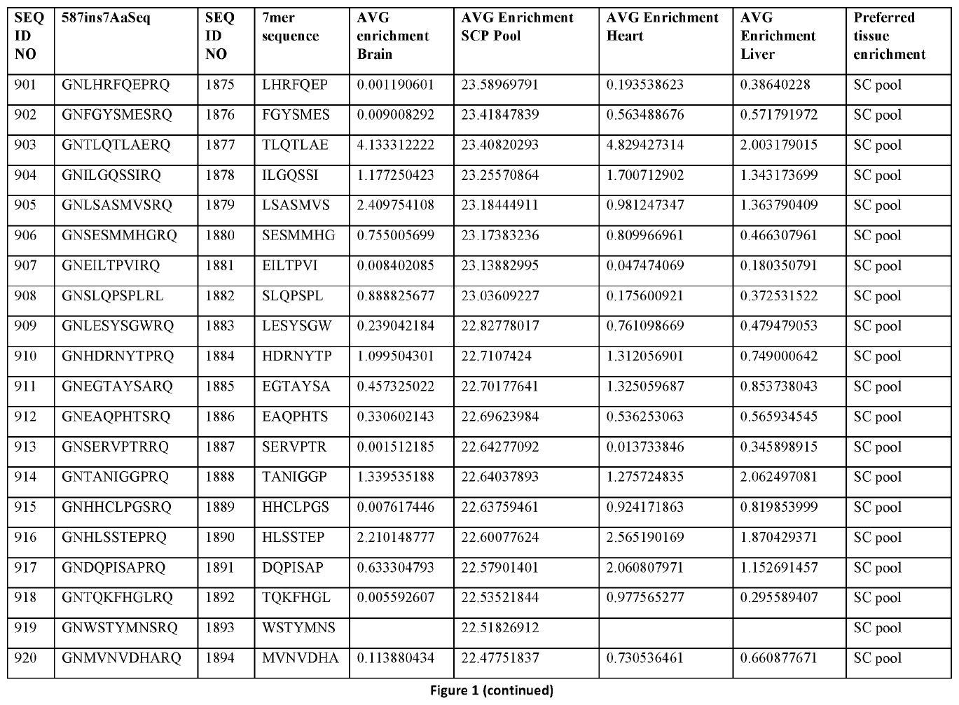

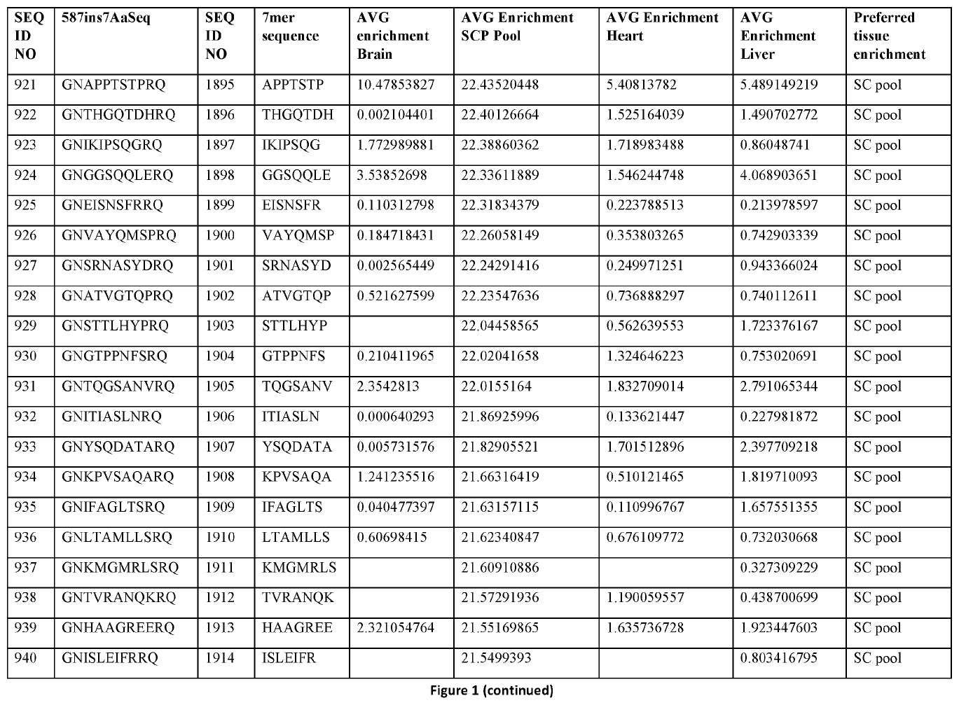

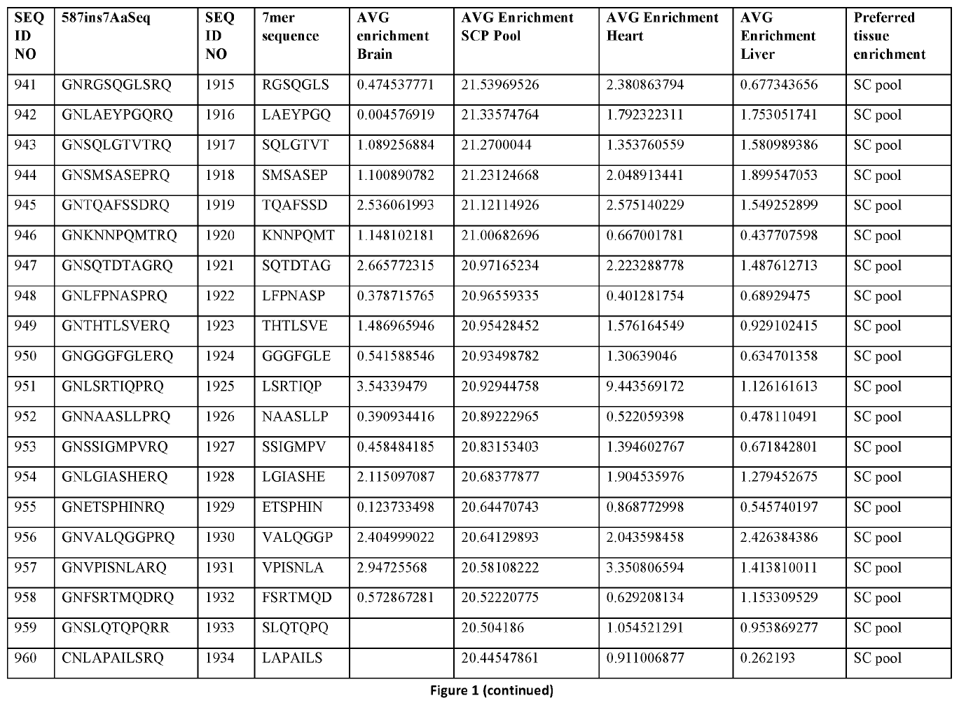

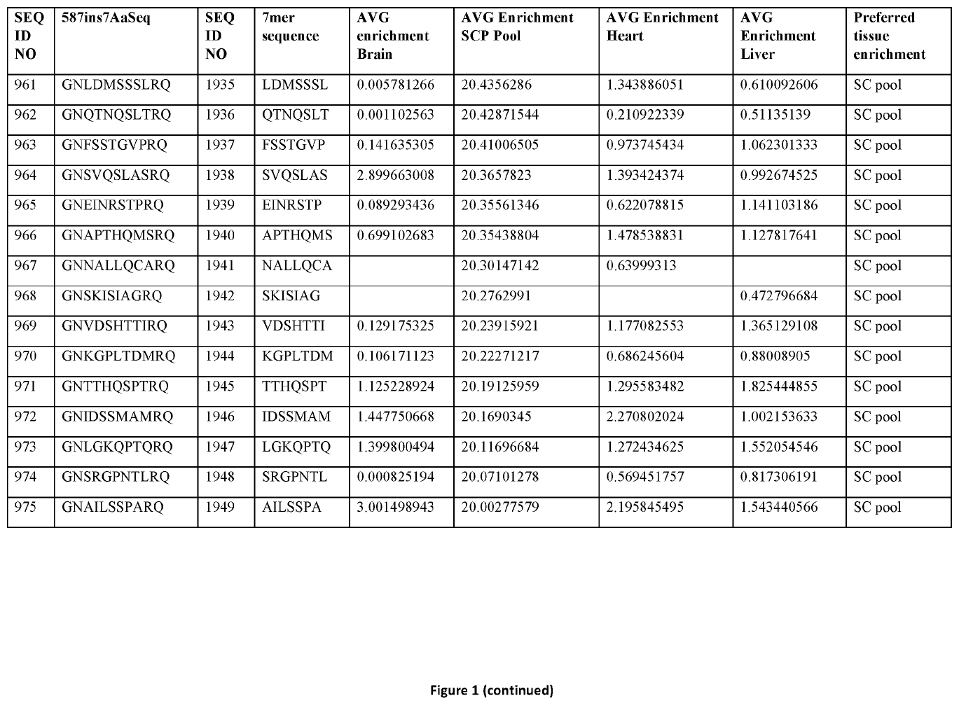

In one aspect, the disclosure provides modified capsid proteins with a peptide insertion and/or substitution comprising or consisting of an amino-acid sequence set forth in any one of Table 1, FIG. 1 and/or Formulas I-III. In certain aspects, the disclosure includes modified capsid proteins with a peptide insertion and/or substitution comprising or consisting of an amino-acid sequence having at least 70% identity with a sequence selected from SEQ ID NOS: 976-1949. In certain aspects, the disclosure includes modified capsid proteins with a peptide insertion and/or substitution comprising or consisting of an amino-acid sequence having at least 80% identity with a sequence selected from SEQ ID NOS: 2-975. In certain aspects, the disclosure includes modified capsid proteins with a peptide insertion and/or substitution comprising or consisting of an amino-acid sequence having at least 90% identity with a sequence selected from SEQ ID NOS: 2-975.

In preferred aspects, the disclosure provides modified capsid proteins with a peptide insertion and/or substitution that confers tissue-specific enrichment. In preferred aspects, the modified capsid proteins provide tissue-specific enrichment in the heart, brain, and/or spinal cord while providing lower enrichment in other tissues, such as the liver. In some embodiments, such a modified capsid protein includes a peptide insertion and/or substitution having an amino acid sequence as described in FIG. 1. In certain aspects, the amino acid sequence as described in FIG. 1 is selected from SEQ ID NOS: 3, 4, 5, and 255. In some embodiments, the amino acid sequence as described in FIG. 1 is selected from SEQ ID NOS: 978, 977, 979, and 1229. In some embodiments, the amino acid sequence as described in FIG. 1 is selected from SEQ ID NOS: 638, 640, and 641. In some embodiments, the amino acid sequence as described in FIG. 1 is selected from SEQ ID NOS: 638, 640, and 641. In some embodiments, the amino acid sequence as described in FIG. 1 is selected from SEQ ID NOS: 1612, 1614, and 1615. In some embodiments, the amino acid sequence as described in FIG. 1 is selected from SEQ ID NOS: 524 and 596. In some embodiments, the amino acid sequence as described in FIG. 1 is selected from SEQ ID NOS: 1498 and 1570

In some embodiments, the insertion sequence is represented by the peptide sequences listed in Table 1.

TABLE 1

| SEQ | 587ins7Aa | SEQ | | AVG Tissue Enrichment | | | |
|---|---|---|---|---|---|---|---|
| ID | Seq | ID | 7 mer | Brain | SCP Pool | Heart | Liver |
| 3 | GNFQLES | 977 | CLCT | 228.4504237 | 113.0057214 | 0.776202543 | 0.417182591 |
| | MNRQ | | FLT | | | | |
| 4 | GNPVAG | 978 | FQLE | 292.989278 | 92.53669424 | 135.7814564 | 0.694118168 |
| | LPPRQ | | SMN | | | | |
| 5 | GNVVSG | 979 | PVAG | 195.9122113 | 85.42798895 | 156.2225293 | 1.167881803 |
| | LAARQ | | LPP | | | | |
| 255 | GNPALYI | 1229 | PALYI | 185.9349112 | 0.494417286 | 0.225254364 | 0.075929853 |
| | FIRQ | | FI | | | | |
| 638 | GNAISNL | 1612 | AISNL | 0.002712026 | | 6.580651162 | 0.354253546 |
| | AKRQ | | AK | | | | |
| 640 | GNCLSSS | 1614 | CLSSS | 1.345246705 | 185.5362984 | 0.449384955 | 0.379868072 |
| | LNRQ | | LN | | | | |
| 641 | GNRAHF | 1615 | RAHF | 7.79751847 | 176.4359943 | | 1.430385742 |
| | LWNRQ | | LWN | | | | |
| 524 | GNMSNL | 1498 | MSNL | 52.42337699 | 0.288265284 | 102.9990962 | 49.85035637 |
| | PPPRQ | | PPP | | | | |
| 596 | GNDNAT | 1570 | DNAT | 0.001028068 | 0.001627494 | 16.71082202 | 1.257996398 |
| | LRPRQ | | LRP | | | | |

“587ins7AaSeq” means the sequence of the 7 mer insert with the surrounding four amino acid residues at the insertion site of residue #587 of SEQ ID NO: 1, with two residues flanking each side of the central 7 mer.
“7 mer” means the sequence of the 7 mer insert without the surrounding insertion site residues.

In certain aspects, the invention provides a modified capsid protein that comprises a seven-amino-acid insertion having a sequence having at least 70% identity with a sequence selected from one of SEQ ID NOS: 976-1949 in an amino acid sequence having a sequence at least 98% identical to amino acid 217 to amino acid 736 of the AAV capsid protein provided in SEQ ID NO: 1.

In certain aspects, the modified AAV capsid protein is characterized by at least one of an increased specificity and/or an increased transduction efficiency when measured in the brain, heart, or spinal cord in a subject when delivered to the subject systemically, relative to a native AAV capsid protein of SEQ ID NO: 1. In certain aspects, the modified AAV capsid protein is characterized by an increased specificity and/or increased transduction efficiency when measured in the brain relative to the capsid protein of SEQ ID NO: 1.

In preferred aspects, the AAV capsid protein is characterized by an increased specificity and/or increased transduction efficiency when measured in the brain relative to the capsid protein of SEQ ID NO: 1. In certain aspects, the seven-amino-acid insertion of a modified capsid protein of the invention comprises a sequence having at least 70% identity with a sequence selected from one of SEQ ID NOS: 976-1411. In certain aspects, AAV capsid proteins of the invention characterized by an increased specificity and/or increased transduction efficiency when measured in the brain include a 7mer amino acid insertion comprising the sequence of Formula [I]:

(SEQ ID NO: 1951)
X¹-X²-X³-X⁴-X⁵-X⁶-X⁷ (Formula [I]),

X¹ is an amino acid selected from L, I, and V; X² is an amino acid selected from L, V, and M; X³ is an amino acid selected from G, S, and P; X⁴ is an amino acid selected from P, G, S, T, and H; X⁵ is an amino acid selected from S, L, T, A, H, and Q; X⁶ is an amino acid selected from L, M, P, S, T, and G; and/or X⁷ is an amino acid selected from S, T, A, L, Q, P, and V.

In some embodiments, the AAV capsid protein comprises an amino acid sequence of formula [I] wherein, wherein X² is L. In some embodiments, the AAV capsid protein comprises an amino acid sequence of formula [I] wherein X³ is G. In some embodiments, the AAV capsid protein comprises an amino acid sequence of formula [I] wherein X⁶ is L. In certain embodiments, the seven-amino-acid insertion is one of SEQ ID NOS: 976-1411. In certain embodiments, the capsid protein comprises a sequence of one of SEQ ID NOS: 2-437.

In preferred aspects, the AAV capsid protein is characterized by an increased specificity and/or increased transduction efficiency when measured in the heart relative to the capsid protein of SEQ ID NO: 1. In certain aspects, the seven-amino-acid insertion of a modified capsid protein of the invention comprises a sequence having at least 70% identity with a sequence selected from one of SEQ ID NOS: 1412-1612. In certain aspects, AAV capsid proteins of the invention characterized by an increased specificity and/or increased transduction efficiency when measured in the heart include a 7mer amino acid insertion comprising the sequence of Formula [II]:

(SEQ ID NO: 1952)
X¹-X²-X³-X⁴-X⁵-X⁶-X⁷ (Formula [II]),

X¹ is an amino acid selected from L, I, and V; X² is an amino acid selected from L, V, and M; X³ is an amino acid selected from G, S, and P; X⁴ is an amino acid selected from P, G, S, T, and H; X⁵ is an amino acid selected from S, L, T, A, H, and Q; X⁶ is an amino acid selected from L, M, P, S, T, and G; and/or X⁷ is an amino acid selected from S, T, A, L, Q, P, and V.

In some embodiments, the AAV capsid protein comprises an amino acid sequence of formula [I] wherein, wherein X² is L. In some embodiments, the AAV capsid protein comprises an amino acid sequence of formula [I] wherein X³ is G. In some embodiments, the AAV capsid protein comprises an amino acid sequence of formula [I] wherein X⁶ is L. In certain embodiments, the seven-amino-acid insertion is one of SEQ ID NOS: 1412-1612. In certain embodiments, the capsid protein comprises a sequence of one of SEQ ID NOS: 438-638.

In preferred aspects, the AAV capsid protein is characterized by an increased specificity and/or increased transduction efficiency when measured in the spinal cord relative to the capsid protein of SEQ ID NO: 1. In certain aspects, the seven-amino-acid insertion of a modified capsid protein of the invention comprises a sequence having at least 70% identity with a sequence selected from one of SEQ ID NOS: 1613-1949. In certain aspects, AAV capsid proteins of the invention characterized by an increased specificity and/or increased transduction efficiency when measured in the spinal cord include a 7mer amino acid insertion comprising the sequence of Formula [III]:

(SEQ ID NO: 1950)
X¹-X²-X³-X⁴-X⁵-X⁶-X⁷ (Formula [III]),

X¹ is an amino acid selected from L, S, and T; X² is an amino acid selected from L, P, S, and T; X³ is an amino acid selected from S, T, G, P, and L; X⁴ is an amino acid selected from S, T, L, and P; X⁵ is an amino acid selected from S, L, T and G; X⁶ is an amino acid selected from L, P, S, and T; and/or X⁷ is an amino acid selected from S, P, and E. In certain embodiments, the seven-amino-acid insertion is one of SEQ ID NOS: 1613-1949. In certain embodiments, the capsid protein comprises a sequence of one of SEQ ID NOS: 639-975.

In preferred aspects, the modified AAV capsid proteins of the invention, comprising an insertion/substitution as described herein, are modified AAV2 capsid proteins.

Also disclosed herein are methods and kits for producing therapeutic recombinant AAV (rAAV) particles, as well as methods and pharmaceutical compositions or formulations comprising the rAAV particles, for the treatment of a disease or condition affecting, for example, the brain, CNS, spinal cord, and/or heart. The rAAV particles of the invention, and pharmaceutical composition or formulations thereof, provide increased specificity and/or increased transduction efficiency compared to rAAVs comprising a native AAV capsid protein sequence. Accordingly, the rAAVs of the present invention find clear utility in treating tissue-specific conditions, e.g., conditions of the brain, spinal cord, and/or heart.

Disclosed herein are AAV capsids engineered with increased viral transduction in the brain, spinal cord, and/or heart. The AAV capsids can encapsidate a viral vector with a heterologous nucleic acid encoding, for example, a therapeutic gene expression product. Transduction of the heterologous nucleic acid in the CNS can be achieved upon systemic delivery to a subject of the AAV capsid of the present disclosure encapsidating a heterologous nucleic acid. The AAV capsids disclosed herein are advantageous for many applications of gene therapy to treat human disease, including, but not limited to, disorders of the brain, spinal cord, and/or heart.

The recombinant AAV vectors comprising a nucleic acid sequence encoding the AAV capsid proteins of the present disclosure as also provided herein. For example, the viral vectors of the present disclosure comprise a nucleic acid sequence comprising the AAV viral Cap (Capsid) encoding VP1, VP2, and VP3, at least one of which is modified to produce the AAV capsid proteins of the present disclosure. The recombinant AAV vector provided can be derived from an AAV serotype (e.g., AAV2) or a variant AAV serotype including an insertion of the present invention. In preferred aspects, the AAV capsid proteins are variants or modifications of an AAV2 capsid protein.

### AAV Capsids

Provided herein are modified adeno-associated (AAV) virus capsid compositions useful for integrating a transgene into a target cell or environment (in a subject when they are administered systemically to the subject.

An rAAV comprises an AAV capsid that can be engineered to encapsidate a heterologous nucleic acid (e.g., therapeutic nucleic acid, gene editing machinery). The AAV capsid is made up of three AAV capsid protein monomers, VP1, VP2, and VP3. Sixty copies of these three VP proteins interact in a 1:1:10 ratio to form the viral capsid. VP1 covers the whole of VP2 protein in addition to a ˜137 amino acid N-terminal region (VP1u), VP2 covers the whole of VP3 in addition to ˜65 amino acid N-terminal region (VP1/2 common region). The three capsid proteins share a conserved amino acid sequence of VP3, which in some cases is the region beginning at amino acid position 138 (e.g., AA139-736).

While not wishing to be bound by theory, it is understood that a parent AAV capsid sequence comprises a VP1 region. In certain embodiments, a parent AAV capsid sequence comprises a VP1, VP2 and/or VP3 region, or any combination thereof. A parent VP1 sequence may be considered synonymous with a parent AAV capsid sequence.

The AAV VP3 structure contains highly conserved regions that are common to all serotypes, a core eight-stranded β-barrel motif βB-βI) and a small α-helix (αA). The loop regions inserted between the β-strands consist of the distinctive HI loop between β-strands H and I, the DE loop between β-strands D and E, and nine variable regions (VRs), which form the top of the loops. These VRs are found on the capsid surface and can be associated with specific functional roles in the AAV life cycle including receptor binding, transduction and antigenic specificity. Residue #587 of the native AAV2 capsid protein (SEQ ID NO: 1) is located at the uppermost portion of one of these variable loop regions.

Accordingly, in some aspects, the rAAV variant of the present invention comprises an AAV capsid protein having a peptide insertion at the residues corresponding to amino acids 587-588 of the AAV2 native sequence of SEQ ID NO: 1.

The AAV capsids comprise AAV capsid proteins (e.g., VP1, VP2, and VP3), each with an insertion, such as in the loop at residue #587 of a parental AAV2 capsid protein structure (AAV2 VP1 numbering). The loop at residue #587 contains the site of heparan sulfate proteoglycan (HSPG) binding of AAV2 and is amenable to peptide display. HSPG is the primary receptor for AAV2, but many indications point toward there being others. Modifications to AAV2 at the loop at residue #587 (the “587 loop”) are shown herein to confer an increased transgene transduction in target in vivo environments, in a tissue-specific manner.

The present invention provides, in an aspect, a peptide insertion at the AAV 587 loop comprising or consisting of an amino-acid sequence set forth in any one of Table 1, FIG. 1 and/or Formulas I-III.

Disclosed herein are AAV capsids comprising AAV capsid proteins with an insertion at the 587 loop that confer a higher enrichment for transduction in CNS cell types (e.g., brain endothelial cells, neurons, astrocytes). In particular, certain AAV capsids of the invention specifically target certain types of CNS cell type, such as brain and/or spinal cord cells. In certain aspects, certain AAV capsids of the invention specifically target cells of the heart.

In particular, the AAV capsid proteins disclosed herein enable rAAV-mediated transduction of a heterologous nucleic acid (e.g., a transgene) in the certain types of cells of a subject. In preferred aspects, the cell type is selected from at least one of cells of the brain, cells of the spinal cord, and/or cells of the heart. The AAV capsids of the present disclosure may be formulated as a pharmaceutical composition. In addition, the AAV capsids can be isolated and purified to be used for a variety of applications, while targeting specific cell types.

In some embodiments, the rAAV capsid of the present disclosure are generated using the methods disclosed herein. In some instances, the rAAV capsid is chimeric. In some instances, the rAAV, or variant AAV protein comprises therein, confer an increase in a localization of the rAAV within the target tissue (e.g., the brain, spinal cord, and/or heart), as compared to the parental AAV capsid or capsid protein.

### AAV Capsid Proteins

Disclosed herein are recombinant AAV (rAAV) capsids which comprise AAV capsid proteins that are engineered with a modified capsid protein (e.g., VP1, VP2, VP3). In some embodiments, the rAAV capsid proteins of the present disclosure are generated using the methods disclosed herein. In some embodiments, the AAV capsid proteins are used in the methods of delivering a therapeutic nucleic acid (e.g., a transgene) to a subject. In some instances, the rAAV capsid proteins have desired AAV expression rendering them particularly suitable for certain therapeutic applications, e.g., the treatment of a disease or disorder in a subject such as those disclosed herein.

Certain rAAV capsid proteins are engineered for optimized expression in the CNS, for example the brain and/or spinal cord, of a subject upon systemic administration of the rAAV to the subject. Certain rAAV capsid proteins are engineered for optimized expression in the heart of a subject upon systemic administration of the rAAV to the subject. In preferred aspects, the rAAV capsid proteins are engineered for reduced expression in liver cells compared to other cell types, in particular, the brain, spinal cord, and/or heart.

The rAAV capsid proteins as described are engineered to include the insertions provided in Table 1, FIG. 1 and/or Formulas I-III. The rAAV capsid proteins including the insertions provided in Table 1, FIG. 1 and/or Formulas I-III are engineered to achieve efficient transduction of an encapsidated transgene. In particular, the rAAV capsid proteins have an increased expression enrichment in the brain, spinal cord, and/or heart of a subject.

The engineered AAV capsid proteins described herein have, in some cases, an insertion of an amino acid that is heterologous to the parental AAV capsid protein at amino acid positions in the 587 loop. In some embodiments, the amino acid is not endogenous to the parental AAV capsid protein at the amino acid position of the insertion. The amino acid may be a naturally occurring amino acid in the same or equivalent amino acid position as the insertion of the substitution in a different AAV capsid protein.

Generally, the insertion comprises a five-, six-, or seven-amino acid sequence (5-mer, 6-mer, or 7-mer, respectively) that is inserted or substituted at the 587 loop in a parental AAV capsid protein. Aspects provided herein provide amino acid insertions comprising seven amino acid polymer (7-mer) inserted at AA587-588, and may additionally include a substitution of one or two amino acids at amino acid positions flanking the 7-mer sequence (e.g., AA586-587 and/or AA588-589) to produce an eleven amino acid polymer (11-mer) at the 587 loop of a parental AAV capsid protein. The 7-mers described herein were advantageously generated using polymerase chain reaction (PCR) with degenerate primers, where each of the seven amino acids is encoded by a deoxyribose nucleic acid (DNA) sequence N-N-K. “N” is any of the four DNA nucleotides and K is guanine (G) or thymine (T). This method of generating random 7-mer amino acid sequences enables 1.28 billion possible combinations at the protein level.

The rAAV capsid proteins of the present disclosure comprise an insertion of an amino acid in an amino acid sequence of an AAV capsid protein. The AAV capsid, from which an engineered AAV capsid protein of the present disclosure is produced, is referred to as a “parental” AAV capsid. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562.

In some cases, the parental AAV is derived from an AAV with a serotype selected from AAV1, AAV2, AAV3, AAV4, AAV5, AAV6, AAV7, AAV8, AAV9, AAV10, AAV11 and AAV12. The AAV capsid protein that is “derived” from another may be a variant AAV capsid protein. A variant may include, for example, a heterologous amino acid in an amino acid sequence of the AAV capsid protein. The heterologous amino acid may be non-naturally occurring in the AAV capsid protein. The heterologous amino acid may be naturally occurring in a different AAV capsid protein. In some instances, the parental AAV capsid is described in US Pat Publication 2020/0165576 and U.S. Pat. App. Ser. No. 62/832,826 and PCT/US20/20778; the content of each of which is incorporated herein.

In some instances, the parental AAV is AAV2. In some instances, the amino acid sequence of the AAV2 capsid protein comprises SEQ ID NO: 1. The amino acid sequence of AAV2 VP1 capsid protein is provided in SEQ ID NO: 1 (MAADGYLPDWLEDTLSEGIRQWWKLKPGPPPPKPAERHKDDSRGLVLPGYKYLGPFN GLDKGEPVNEADAAALEHDKAYDRQLDSGDNPYLKYNHADAEFQERLKEDTSFGGNL GRAVFQAKKRVLEPLGLVEEPVKTAPGKKRPVEHSPVEPDSSSGTGKAGQQPARKRLNF GQTGDADSVPDPQPLGQPPAAPSGLGTNTMATGSGAPMADNNEGADGVGNSSGNWHC DSTWMGDRVITTSTRTWALPTYNNHLYKQISSQSGASNDNHYFGYSTPWGYFDFNRFH CHFSPRDWQRLINNNWGFRPKRLNFKLFNIQVKEVTQNDGTTTIANNLTSTVQVFTDSE YQLPYVLGSAHQGCLPPFPADVFMVPQYGYLTLNNGSQAVGRSSFYCLEYFPSQMLRT GNNFTFSYTFEDVPFHSSYAHSQSLDRLMNPLIDQYLYYLSRTNTPSGTTTQSRLQFSQA GASDIRDQSRNWLPGPCYRQQRVSKTSADNNNSEYSWTGATKYHLNGRDSLVNPGPA MASHKDDEEKFFPQSGVLIFGKQGSEKTNVDIEKVMITDEEEIRTTNPVATEQYGSVSTN LQRG*NR*QAATADVNTQGVLPGMVWQDRDVYLQGPIWAKIPHTDGHFHPSPLMGGF GLKHPPPQILIKNTPVPANPSTTFSAAKFASFITQYSTGQVSVEIEWELQKENSKRWNPEI QYTSNYNKSVNVDFTVDTNGVYSEPRPIGTRYLTRNL). In some instances, the parental AAV capsid protein sequence is 70%, 75%, 80%, 85%, 90%, 91%, 92%, 93%, 94%, 95%, 96%, 97%, 98%, 99%, or 100% homologous to SEQ ID NO: 1. The 7-mer insertion is located between the *NR*.

AAV capsid proteins from native AAV serotypes, such as AAV2, with tropisms activate the innate immune response, which in some cases causes a severe inflammatory response in a subject, which can lead to multi-organ failure, including the liver. By improving transduction enrichment of a native AAV serotype for a target in vivo tissue (e.g., brain, spinal cord, and/or heart), the rAAV particles of the present disclosure reduce the immunogenic properties of AAV-mediated transgene delivery and prevent activation of the innate immune response.

In some instances, the parental AAV capsid protein comprises the entire VP1 region provided in SEQ ID NO: 1 (e.g., amino acids 1-736). In some instances, the parental AAV capsid protein comprises amino acids 217-736 in SEQ ID NO: 1, which is the common region found in VP1, VP2 and VP3 AAV9 capsid proteins. In some instances, the AAV capsid protein comprises amino acids 64-736 in SEQ ID NO: 1, which is the common region found in VP1 and VP2. The parental AAV capsid protein sequence may comprise amino acids selected from 1-736, 10-736, 20-736, 30-736, 40-736, 50-736, 60-736, 70-736, 80-736, 90-736, 100-736, 110-736, 120-736, 130-736, 140-736, 150-736, 160-736, 170-736, 180-736, 190-736, 200-736, 210-736, 220-736, 230-736, 240-736, 250-736, 260-736, 270-736, 280-736, 290-736, 300-736, 310-736, 320-736, 330-736, 340-736, 350-736, 360-736, 370-736, 380-736, 390-736, 400-736, 410-736, 420-736, 430-736, 440-736, and 450-736, from SEQ ID NO: 1. In some aspects, the rAAV variant comprises an AAV capsid protein comprising an amino acid sequence that is at least 98% identical to amino acid 217 to amino acid 736 of SEQ ID NO: 1. In some instances, the amino acid insertion is at a three (3)-fold axis of symmetry of a corresponding parental AAV capsid protein.

Disclosed herein are insertions of an amino acid sequence in an AAV capsid protein. Where the sequence numbering designation “587-588” is noted for AAV2, for example AAV VP1, the invention also includes insertions in similar locations in the other AAV serotypes. As used herein, “AA587-588” indicates that the insertion of the amino acid (or amino acid sequence) is immediately after an amino acid (AA) at position 587 and immediately before an AA at position 588 within an amino acid sequence of a parental AAV VP capsid protein (VP1 numbering).

It is envisioned that the insertions disclosed herein (Table 1, FIG. 1 and/or Formulas I-III) may be inserted at AA587-588 in an amino acid sequence of a parental AAV2 capsid protein, a variant thereof, or equivalent amino acid position of a parental AAV of a different serotype (e.g., AAV1, AAV3, AAV9, and the like).

The insertions described herein may, in some cases, comprise a 7-mer insertion at AA587-588. It is envisioned that any 7-mer insertion disclosed herein in addition to a substitution with any amino acid at amino acid positions 586-589 may comprise an 11-mer.

Disclosed herein are AAV capsid proteins with an insertion described above in a parental AAV capsid protein that confers an increased transduction enrichment in the CNS in a subject, even when delivered systemically. One of the many advantages of certain AAV capsid proteins described herein is their ability to target tissue and cells within the CNS. The tissue can be the brain. Non-limiting examples of CNS cells include a neuron and a glial cell. Glial cells can be selected from an oligodendrocyte, an ependymal cell, an astrocyte and a microglia. In preferred aspects, the AAV capsid proteins described herein target tissues and cells of the brain and/or spinal cord. In certain aspects, the AAV capsid proteins described herein target tissues and cells of the heart.

In some instances, the AAV capsid protein comprises an insertion of at least or about five, six, or seven amino acids of an amino acid sequence of Table 1, FIG. 1 and/or Formulas I-III at an amino acid position 587-588 in a parental AAV9 capsid protein (SEQ ID NO: 1). In some cases, the AAV capsid protein has increased viral transduction in brain.

The rAAV capsid proteins of the present disclosure may also have a substitution of an amino acid sequence at amino acid position 452-458 in a parental AAV2 capsid protein, or variant thereof, as described in WO2020068990.

The rAAV capsid proteins described herein may be isolated and purified. The AAV may be isolated and purified by methods standard in the art such as by column chromatography, iodixanol gradients, or cesium chloride gradients. Methods for purifying AAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

In addition, the AAV capsid proteins disclosed herein, either isolated and purified, or not, may be formulated into a pharmaceutical formulation, which in some cases, further comprises a pharmaceutically acceptable carrier.

The rAAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein.

“Percent Identity” is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see: Henikoff and Henikoff, (1989) Proc. Natl. Acad. Sci. USA 89: 10915).

Sequence identity/similarity values provided herein can refer to the value obtained using the BLAST+ 2.5.0 suite of programs using default settings (blast.ncbi.nlm.nih.gov) (Camacho, C., et al. (2009) BLAST+: architecture and applications. BMC Bioinformatics 10:421).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17: 149-63) and XNU (Ci-ayerie and States (1993) Comput. Chem. 17: 191-201) low-complexity filters can be employed alone or in combination.

The terms “substantial identity” and “substantially identical” indicate that a polypeptide or nucleic acid comprises a sequence with between 55-100% sequence identity to a reference sequence, with at least 55% sequence identity, or at least 60%, or at least 65%, or at least 70%, or at least 75%, or at least 80%, or at least 85%, or at least 90%, or at least 95%, or at least 99% sequence identity or any percentage of value within the range of 55-100% sequence identity relative to the reference sequence. The percent sequence identity may occur over a specified comparison window. Optimal alignment may be ascertained or conducted using the homology alignment algorithm of Needleman and Wunsch, supra.

For example, the insertion sequences may include, but are not limited to, sequences that are not exactly the same as the sequences disclosed herein, but which have, in addition to the substitutions explicitly described for various sequences listed herein, additional substitutions of amino acid residues which substantially do not impair the activity or properties of the sequences described herein, such as those predicted by homology software e.g. BLOSUM62 matrices.

### AAV Particles

The rAAV particles with the insertion sequences described herein have an increased transduction enrichment in the brain, spinal cord, and/or heart. In some instances, the increased transduction enrichment comprises a 1-fold, 2-fold, 3-fold, 4-fold, 5-fold, 6-fold, 7-fold, 8-fold, 9-fold or 10-fold increase, or more relative to rAAV particles with a native AAV2 capsid protein (SEQ ID NO: 1). In some instances, the increased transduction enrichment is at least 1-fold. In some instances, the increased transduction enrichment is at least 2-fold. In some instances, the increased transduction enrichment is at least 4-fold.

The rAAV particles with the insertion sequences described herein have an increased expression enrichment in the brain, spinal cord, and/or heart. Detecting whether a rAAV possesses more or less expression, includes measuring a level of gene expression product (e.g., RNA or protein) expressed from the heterologous nucleic acid encapsidated by the rAAV in a tissue sample obtained from a subject. Suitable methods for measuring expression of a gene expression product include next-generation sequencing (NGS) and quantitative polymerase chain reaction (qPCR).

The increased expression in cells of the brain, spinal cord, and/or heart is represented by the enrichment values provided in FIG. 1. FIG. 1 also provides expression levels in the liver, which for many sequences, are far lower than the values for the brain, spinal cord, and/or heart. This reveals the tissue- or cell-type specificity conferred by the modified capsid proteins and rAAVs disclosed herein.

### Heterologous Nucleic Acids

Disclosed herein are therapeutic nucleic acids useful for the treatment or prevention of a disease or condition, or symptom of the disease or condition. In some embodiments, the therapeutic nucleic acids encode a therapeutic gene expression product. Non-limiting examples of gene expression products include proteins, polypeptides, peptides, enzymes, antibodies, antigen binding fragments, nucleic acid (RNA, DNA, antisense oligonucleotide, siRNA, and the like), and gene editing components, for use in the treatment, prophylaxis, and/or amelioration of the disease or disorder, or symptoms of the disease or disorder. In some instances, the therapeutic nucleic acids are placed in an organism, cell, tissue or organ of a subject by way of a rAAV, such as those disclosed herein.

Disclosed herein are rAAVs, each comprising a viral vector (e.g., a single stranded DNA molecule (ssDNA)). In some instances, the viral vector comprises two inverted terminal repeat (ITR) sequences that are about 145 bases each, flanking a transgene. In some embodiments, the transgene comprises a therapeutic nucleic acid, and in some cases, a promoter in cis with the therapeutic nucleic acid in an open reading frame (ORF). The promoter is capable of initiating transcription of therapeutic nucleic acid in the nucleus of the target cell. The ITR sequences can be from any AAV serotype. Non-limiting examples of AAV serotypes include AAV1, AAV2, AAV3, AAV4, AAV5, AAV6, AAV7, AAV8, AAV9, AAV10, AAV11, and AAV12. In some cases, an ITR is from AAV2. In some cases, an ITR is from AAV9.

Disclosed herein are transgenes that can comprise any number of nucleotides. In some cases, a transgene can comprise less than about 100 nucleotides. In some cases, a transgene can comprise at least about 100 nucleotides. In some cases, a transgene can comprise at least about 200 nucleotides. In some cases, a transgene can comprise at least about 300 nucleotides. In some cases, a transgene can comprise at least about 400 nucleotides. In some cases, a transgene can comprise at least about 500 nucleotides. In some cases, a transgene can comprise at least about 1000 nucleotides. In some cases, a transgene can comprise at least about 5000 nucleotides. In some cases, a transgene can comprise over 5,000 nucleotides. In some cases, a transgene can comprise between about 500 and about 5000 nucleotides. In some cases, a transgene comprises about 5000 nucleotides. In any of the cases disclosed herein, the transgene can comprise DNA, RNA, or a hybrid of DNA and RNA. In some cases, the transgene can be single stranded. In some cases, the transgene can be double stranded.

Disclosed herein are transgenes useful for modulating the expression or activity of a target gene or gene expression product thereof. In some instances, the transgene is encapsidated by an rAAV capsid protein of an rAAV particle described herein. In some instances, the rAAV particle is delivered to a subject to treat a disease or condition disclosed herein in the subject. In some instances, the delivery is systemic.

The transgenes disclosed herein are useful for expressing an endogenous gene at a level similar to that of a healthy or normal individual. This is particularly useful in the treatment of a disease or condition related to the underexpression, or lack of expression, of a gene expression product. In some embodiments, the transgenes disclosed herein are useful for overexpressing an endogenous gene, such that an expression level of the endogenous gene is above the expression level of a healthy or normal individual. Additionally, transgenes can be used to express exogenous genes (e.g., active agent such as an antibody, peptide, nucleic acid, or gene editing components). In some embodiments, the therapeutic gene expression product is capable of altering, enhancing, increasing, or inducing the activity of one or more endogenous biological processes in the cell. In some embodiments, the transgenes disclosed herein are useful for reducing expression of an endogenous gene, for example, a dominant negative gene. In some embodiments, the therapeutic gene expression product is capable of altering, inhibiting, reducing, preventing, eliminating, or impairing the activity of one or more endogenous biological processes in the cell. In some aspects, the increase of gene expression refers to an increase by at least about 20%, 30%, 40%, 50%, 60%, 70%, 80%, 85%, 90%, 95% and 100%. In one aspect, the protein product of the targeted gene may be increased by at least about 20%, 30%, 40%, 50%, 60%, 70%, 80%, 85%, 90%, 95% and 100%. In some aspects, the decrease of gene expression refers to an increase by at least about 20%, 30%, 40%, 50%, 60%, 70%, 80%, 85%, 90%, 95% and 100%. In one aspect, the protein product of the targeted gene may be decreased by at least about 20%, 30%, 40%, 50%, 60%, 70%, 80%, 85%, 90%, 95% and 100%.

When endogenous sequences (endogenous or part of a transgene) are expressed with a transgene, the endogenous sequences can be full-length sequences (wild-type or mutant) or partial sequences. The endogenous sequences can be functional. Non-limiting examples of the function of these full length or partial sequences include increasing the serum half-life of the polypeptide expressed by a transgene (e.g., therapeutic gene) and/or acting as a carrier.

A transgene can be inserted into an endogenous gene such that all, some or none of the endogenous gene is expressed. For example, a transgene as described herein can be inserted into an endogenous locus such that some (N-terminal and/or C-terminal to a transgene) or none of the endogenous sequences are expressed, for example as a fusion with a transgene. In other cases, a transgene (e.g., with or without additional coding sequences of the endogenous gene) is integrated into any endogenous locus, for example a safe-harbor locus. For example, a Frataxin (FXN) transgene can be inserted into an endogenous FXN gene. A transgene can be inserted into any gene, e.g., the genes as described herein.

At least one advantage of the present disclosure is that virtually any therapeutic nucleic acid may be used to express any therapeutic gene expression product. In some instances, the therapeutic gene expression product is a therapeutic protein or a peptide (e.g., antibody, antigen-binding fragment, peptide, or protein). In one embodiment the protein encoded by the therapeutic nucleic acid is between 50-5000 amino acids in length. In some embodiments the protein encoded is between 50-2000 amino acids in length. In some embodiments the protein encoded is between 50-1000 amino acids in length. In some embodiments the protein encoded is between 50-1500 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length. In some embodiments the peptide encoded is between 4-50 amino acids in length. In some embodiments, the protein encoded is a tetrapeptide, a pentapeptide, a hexapeptide, a heptapeptide, an octapeptide, a nonapeptide, or a decapeptide. In some embodiments, the protein encoded comprises a peptide of 2-30 amino acids, such as for example 5-30, 10-30, 2-25, 5-25, 10-25, or 10-20 amino acids. In some embodiments, the protein encoded comprises a peptide of at least 11, 12, 13, 14, 15, 17, 20, 25 or 30 amino acids, or a peptide that is no longer than 50 amino acids, e.g., no longer than 35, 30, 25, 20, 17, 15, 14, 13, 12, 11 or 10 amino acids.

Non-limiting examples of therapeutic protein or peptides include an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphoring, a semaphorin receptor, a serotonin transport protein, a serotonin uptake inhibitor, a serotonin receptor, a serpin, a serpin receptor, and a tumor suppressor. In certain embodiments, the therapeutic protein or peptide is selected from brain-derived neurotrophic factor (BDNF), ciliary neurotrophic factor (CNTF), macrophage colony-stimulating factor (CSF), epidermal growth factor (EGF), fibroblast growth factor (FGF), gonadotropin, interferon-gamma (IFN), insulin-like growth factor 1 (IFG-1), nerve growth factor (NGF), platelet-derived growth factor (PDGF), pigment epithelium-derived factor (PEDF), transforming growth factor (TGF), transforming growth factor-beta (TGF-B), tumor necrosis factor (TNF), vascular endothelial growth factor (VEGF), prolactin, somatotropin, X-linked inhibitor of apoptosis protein 1 (XIAP1), interleukin 1 (IL-1), IL-2, IL-3, IL-4, IL-5, IL-6, IL-7, IL-8, IL-9, IL-10, IL-10, viral IL-10, IL-11, IL-12, IL-13, IL-14, IL-15, IL-16, IL-17, and IL-18.

A therapeutic gene expression product can comprise gene editing components. Non-limiting examples of gene editing components include those required for CRISPR/Cas, artificial site-specific RNA endonuclease (ASRE), zinc finger endonuclease (ZFN), and transcription factor like effector nuclease (TALEN). In a non-limiting example, a subject having Huntington's disease is identified. The subject is then systemically administered a first amount of a rAAV encapsidating a viral vector encoding ZFN engineered to represses the transcription of the Huntingtin (HTT) gene. The rAAV will include a modified AAV capsid protein that includes an amino acid sequence provided in any one of Table 1, FIG. 1 and/or Formulas I-III, so as to allow proper targeting of the ZFN to the brain, while reducing expression in off-target organs, such as the liver. If needed, the subject is administered a second or third dose of the rAAV, until a therapeutically effective amount of the ZFN is expressed in the subject's brain.

A therapeutic nucleic acid can comprise a non-protein coding gene e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. The non-protein coding gene may also encode a tRNA, rRNA, tmRNA, piRNA, double stranded RNA, snRNA, snoRNA, and/or long non-coding RNA (lncRNA). In some cases, the non-protein coding gene can modulate the expression or the activity of a target gene or gene expression product. For example, the RNAs described herein may be used to inhibit gene expression in the CNS. In some cases, inhibition of gene expression refers to an inhibition by at least about 20%, 30%, 40%, 50%, 60%, 70%, 80%, 85%, 90%, 95% and 100%. In some cases, the protein product of the targeted gene may be inhibited by at least about 20%, 30%, 40%, 50%, 60%, 70%, 80%, 85%, 90%, 95% and 100%. The gene can be either a wild type gene or a gene with at least one mutation. The targeted protein may be either a wild type protein or a protein with at least one mutation.

A therapeutic nucleic acid can modulate the expression or activity of a gene or gene expression product expressed from the gene that is implicated in a disease or disorder of the brain, spinal cord, and/or heart. For example, the therapeutic nucleic acid, in some cases is a gene or a modified version of the gene described herein. In some instances, the gene or gene expression product is inhibited. In some instances, the gene or gene expression product is enhanced.

In another example, the therapeutic nucleic acid comprises an effector gene expression product such as a gene editing component specific to target a gene therein. Non-limited examples of genes include target gene or gene expression product selected from ATP1A2, CACNAIA, SETD5, SHANK3, NF2, DNMT1, TCF4, RAI1, PEX1, ARSA, EIF2B5, EIF2B1, EIF2B2, NPC1, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMNI), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SODI), iduronate 2 sulfatase (hIDS), Glucosylceramidase Beta (GBA), fragile X mental retardation 1 (FMRI), NPC Intracellular Cholesterol Transporter 1 (NPCl), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11, PEX12, PEX13, PEX14, PEX16, PEX19, and PEX26. In some instances, the gene or gene expression product is inhibited. In some instances, the gene or gene expression product is enhanced.

### AAV Vectors

Aspects disclosed herein comprise plasmid vectors comprising a nucleic acid sequence encoding the AAV capsids and AAV capsid proteins described herein. AAV vectors described herein are useful for the assembly of a rAAV and viral packaging of a heterologous nucleic acid. In addition, an AAV vector may encode a transgene comprising the heterologous nucleic acid.

An AAV vector can comprise a transgene, which in some cases encodes a heterologous gene expression product (e.g., therapeutic gene expression product, recombinant capsid protein, and the like). The transgene is in cis with two inverted terminal repeats (ITRs) flanking the transgene. The transgene may comprise a therapeutic nucleic acid encoding a therapeutic gene expression product. Due to the limited packaging capacity of the rAAV (˜5 kB), in some cases, a longer transgene may be split between two AAV vectors, the first with 3′ splice donor and the second with a 5′ splice acceptor. Upon co-infection of a cell, concatemers form, which are spliced together to express a full-length transgene.

A transgene is generally inserted so that its expression is driven by the endogenous promoter at the integration site, namely the promoter that drives expression of the endogenous gene into which a transgene is inserted. In some instances, a transgene comprises a promoter and/or enhancer, for example a constitutive promoter or an inducible or tissue/cell specific promoter. As a non-limiting example, the promoter may be CMV promoter, a CMV-3-Actin-intron-β-Globin hybrid promoter (CAG), CBA promoter, FRDA or FXN promoter, UBC promoter, GUSB promoter, NSE promoter, Synapsin promoter, MeCP2 promoter, GFAP promoter, H1 promoter, U6 promoter, NFL promoter, NFH promoter, SCN8A promoter, or PGK promoter. As a non-limiting example, promoters can be tissue-specific expression elements include, but are not limited to, human elongation factor 1α-subunit (EF1α), immediate-early cytomegalovirus (CMV), chicken β-actin (CBA) and its derivative CAG, the β glucuronidase (GUSB), and ubiquitin C (UBC). The transgene may include a tissue-specific expression elements for neurons such as, but not limited to, neuron-specific enolase (NSE), platelet-derived growth factor (PDGF), platelet-derived growth factor B-chain (PDGF-β), the synapsin (Syn), the methyl-CpG binding protein 2 (MeCP2), Ca2+/calmodulin-dependent protein kinase II (CaMKII), metabotropic glutamate receptor 2 (mGluR2), NFL, NFH, np32, PPE, Enk and EAAT2 promoters. The transgene may comprise a tissue-specific expression element for astrocytes such as, but not limited to, the glial fibrillary acidic protein (GFAP) and EAAT2 promoters. The transgene may comprise tissue-specific expression elements for oligodendrocytes such as, but not limited to, the myelin basic protein (MBP) promoter.

In some embodiments, the promoter is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. The promoter may provide expression of the therapeutic gene expression product for a period of time in targeted tissues such as, but not limited to, the CNS. Expression of the therapeutic gene expression product may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years. Expression of the payload may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years or 10-15 years, or 15-20 years, or 20-25 years, or 25-30 years, or 30-35 years, or 35-40 years, or 40-45 years, or 45-50 years, or 50-55 years, or 55-60 years, or 60-65 years.

An AAV vector can comprise a genome of a helper virus. Helper virus proteins are required for the assembly of a recombinant AAV (rAAV), and packaging of a transgene containing a heterologous nucleic acid into the rAAV. The helper virus genes are adenovirus genes E4, E2a and VA, that when expressed in the cell, assist with AAV replication. In some embodiments, an AAV vector comprises E2. In some embodiments, an AAV vector comprises E4. In some embodiments, an AAV vector comprises VA. In some instances, the AAV vector comprises one of helper virus proteins, or any combination.

The target gene or gene expression product for use in a transgene can be selected from ATP1A2, CACNAIA, SETD5, SHANK3, NF2, DNMT1, TCF4, RAI1, PEX1, ARSA, EIF2B5, EIF2B1, EIF2B2, NPC1, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMNI), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SOD1), iduronate 2 sulfatase (hIDS), Glucosylceramidase Beta (GBA), fragile X mental retardation 1 (FMRI), NPC Intracellular Cholesterol Transporter 1 (NPCl), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX1, PEX12, PEX13, PEX14, PEX16, PEX19, and PEX26.

An AAV vector can comprise a viral genome comprising a nucleic acid encoding the recombinant AAV (rAAV) capsid protein described herein. The viral genome can comprise a Replication (Rep) gene encoding a Rep protein, and Capsid (Cap) gene encoding an AAP protein in the first open reading frame (ORF1) or a Cap protein in the second open reading frame (ORF2). The Rep protein is selected from Rep78, Rep68, Rep52, and Rep40. In some instances, the Cap gene is modified encoding a modified AAV capsid protein described herein. A wild-type Cap gene encodes three proteins, VP1, VP2, and VP3. In some cases, VP1 is modified. In some cases, VP2 is modified. In some cases, VP3 is modified. In some cases, all three VP1-VP3 are modified. The AAV vector can comprise nucleic acids encoding wild-type Rep78, Rep68, Rep52, Rep40 and AAP proteins.

In some instances, the AAV2 VP1 gene provided in SEQ ID NO: 1, which is modified to include any one of SEQ ID NOS: 2-1949 in FIG. 1. The AAV vector described herein may be used to produce a variant AAV capsid by the methods described herein.

### Methods of Producing rAAVs

Disclosed herein are methods of producing the AAV capsids comprising the AAV capsid proteins and viral vector encoding a therapeutic nucleic acid. The AAV capsid proteins are produced by introducing into a cell (e.g., immortalized stem cell) a first vector containing a transgene cassette flanked by inverted terminal repeat (ITR) sequences from a parental AAV virus (the transgene cassette has a promoter sequence that drives transcription of a heterologous nucleic acid in the nucleus of the target cell), a second vector encoding the AAV genome with a AAV capsid protein (encoding the AAV Rep gene as well as the modified Cap gene for the variant being produced), and a third vector encoding helper virus proteins, required for assembly of the AAV capsid structure and packaging of the transgene in the modified AAV capsid structure. The assembled AAV capsid can be isolated and purified from the cell using suitable methods known in the art.

The transgenes contained in a recombinant AAV (rAAV) vector and encapsidated by the AAV capsid proteins of the present disclosure are also provided herein. The transgenes disclosed herein are delivered to a subject for a variety of purposes, such as to treat a disease or condition in the subject. The transgene can be gene editing components that modulate the activity or expression of a target gene or gene expression product. Alternatively, the transgene is a gene encoding a therapeutic gene expression product that is effective to modulate the activity or expression of itself, or another target gene or gene expression product.

Aspects disclosed herein provide methods of manufacturing rAAV virus or virus particles comprising: (a) introducing into a cell a nucleic acid comprising: (i) first vector containing a transgene cassette flanked by inverted terminal repeat (ITR) sequences from a parental AAV virus (the transgene cassette has a promoter sequence that drives transcription of a heterologous nucleic acid in the nucleus of the target cell); (ii) a second vector encoding the AAV genome with a AAV capsid protein of the present invention; and (iii) a vector encoding helper virus proteins, required for assembly of the AAV capsid structure and packaging of the transgene in the modified AAV capsid structure; (b) expressing in the cell the AAV capsid protein described herein; (c) assembling an AAV particle comprising the AAV capsid proteins disclosed herein; and (d) packaging the AAV particle. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the Cap gene is derived from the deoxyribose nucleic acid (DNA) provided in SEQ ID NO: 86. In some instances, the 5′ ITR and the 3′ ITR are derived from an AAV2 serotype. In some instances, the 5′ ITR and the 3′ ITR are derived from an AAV5 serotype. In some instances, the 5′ ITR and the 3′ ITR are derived from an AAV9 serotype. In some instances, the first nucleic acid sequence and the second nucleic acid sequence are in trans. In some instances, the first nucleic acid sequence and the second nucleic acid sequence are in cis. In some instances, the first nucleic acid sequence, the second nucleic acid sequence and the third nucleic acid sequence, are in trans.

In some instances, the methods comprise packing the first nucleic acid sequence encoding the therapeutic gene expression product such that it becomes encapsidated by the modified AAV capsid protein. In some embodiments, the rAAV particles are isolated, concentrated, and purified using suitable viral purification methods, such as those described herein.

In some cases, rAAVs of the present disclosure are generated using the methods described in Challis, R. C. et al. Nat. Protoc. 14, 379 (2019). Briefly, triple transfection of HEK293T cells (ATCC) using polyethylenimine (PEI) is performed, viruses are collected after 120 hours from both cell lysates and media and purified over iodixanol. In a non-limiting example, the rAAVs are generated by triple transfection of precursor cells (e.g., HEK293T) cells using a standard transfection protocol (e.g., PEI). Viral particles are harvested from the media after a period of time (e.g., 72 h post transfection) and from the cells and media at a later point in time (e.g., 120 h post transfection). Virus present in the media is concentrated by precipitation with 8% polyethylene glycol (PEG) and 500 mM sodium chloride and the precipitated virus is added to the lysates prepared from the collected cells. The viruses are purified over iodixanol (Optiprep, Sigma) step gradients (15%, 25%, 40% and 60%). Viruses are concentrated and formulated in PBS. Virus titers are determined by measuring the number of DNaseI-resistant vector genome copies (VGs) using qPCR and the linearized genome plasmid as a control.

The cell can be selected from a human, a primate, a murine, a feline, a canine, a porcine, an ovine, a bovine, an equine, an epine, a caprine and a lupine host cell. In some instances, the cell is a progenitor or precursor cell, such as a stem cell. In some instances, the stem cell is a mesenchymal cell, embryonic stem cell, induced pluripotent stem cell (iPSC), fibroblast or other tissue specific stem cell. The cell can be immortalized. In some cases, the immortalized cell is a HEK293cell. In some instances, the cell is a differentiated cell. Based on the disclosure provided, it is expected that this system can be used in conjunction with any transgenic line expressing a recombinase in the target cell type of interest to develop AAV capsids that more efficiently transduce that target cell population.

### Methods of Treatment

Disclosed herein are methods of treating a disease or condition, or a symptom of the disease or condition, in a subject, comprising administrating of therapeutically effective amount of one or more compositions (e.g., rAAV particle, AAV vector, pharmaceutical composition) disclosed herein to the subject. In some embodiments, the composition is a rAAV capsid protein described herein. In some embodiments, the composition is an isolated and purified rAAV capsid protein described herein. In some embodiments, the rAAV particle encapsidates an AAV vector comprising a transgene (e.g., therapeutic nucleic acid). In some embodiments, the composition is a rAAV capsid protein described herein conjugated with a therapeutic agent disclosed herein. In some embodiments, the composition is a pharmaceutical composition comprising the rAAV particle and a pharmaceutically acceptable carrier. In some embodiments, the one or more compositions are administered to the subject alone (e.g., stand-alone therapy). In some embodiments, the composition is a first-line therapy for the disease or condition. In some embodiments, the composition is a second-line, third-line, or fourth-line therapy, for the disease or condition.

Recombinant adeno-associated virus (rAAV) mediated gene delivery leverages the AAV mechanism of viral transduction for nuclear expression of an episomal heterologous nucleic acid (e.g., a transgene, therapeutic nucleic acid). Upon delivery to a host in vivo environment, a rAAV will (1) bind or attach to cellular surface receptors on the target cell, (2) endocytose, (3) traffic to the nucleus, (4) uncoat the virus to release the encapsidated heterologous nucleic acid, (5) convert of the heterologous nucleic acid from single-stranded to double-stranded DNA as a template for transcription in the nucleus, and (6) transcribe the episomal heterologous nucleic acid in the nucleus of the host cell (“transduction”). rAAVs engineered to have an increased tissue-specific transduction enrichment (transcription of the episomal heterologous nucleic acid in the host cell) are desirable for gene therapy applications.

Aspects disclosed herein provide methods of treating a disease or condition in a subject, the method comprising administering to the subject a therapeutically effective amount of the rAAV of the present disclosure, or the pharmaceutical formulation of the present disclosure, wherein the gene product is a therapeutic gene product. In some embodiments, the administering is by intracranial, intraventricular, intracerebroventricular, intravenous, intraarterial, intranasal, intrathecal, intracisternae magna, or subcutaneous.

Provided here, are methods of treating a disease or a condition associated with an aberrant expression or activity of a target gene or gene expression product thereof, the method comprising modulating the expression or the activity of a target gene or gene expression product in a subject by administering a rAAV encapsidating a heterologous nucleic acid of the present disclosure. In some instances, the expression or the activity of the target gene or gene expression product is decreased, relative to that in a normal (non-diseased) individual; and administering the rAAV to the subject is sufficient to increase the expression of the activity of the target gene or gene expression product. In some instances, the expression or the activity of the gene or gene expression product is increased, relative to that in a normal individual; and administering the rAAV to the subject is sufficient to decrease the expression or the activity of the target gene or gene expression product. In a non-limiting example, a subject diagnosed with Alzheimer's disease, which is caused, in some cases, by a gain-of-function of a Presenilin 1 and/or Presenilin 2 (encoded by the gene PSEN1 and PSEN2, respectively) is administered a rAAV disclosed herein encapsidating a therapeutic nucleic acid that is a silencing RNA (siRNA), or other RNAi with a loss-of-function effect on PSEN1 mRNA.

Also provided are methods of preventing a disease or condition disclosed herein in a subject comprising administering to the subject a therapeutically effective amount of an rAAV vector comprising a nucleic acid sequence encoding a therapeutic gene expression product described herein. The rAAV vector may be encapsidated in the modified capsid protein or rAAV viral particle described herein. In some instances, the therapeutic gene expression product is effective to modulate the activity or expression of a target gene or gene expression product.

Disclosed herein are methods of treating a disease or condition in a subject by administering a composition comprising a rAAV disclosed herein. An advantage of the rAAVs disclosed herein, is that the rAAV may be used to treat virtually any disease or condition that would benefit from a transgene therapy, including but not limited to spinal muscular atrophy (SMA), amyotrophic lateral sclerosis (ALS), Parkinson's disease, Pompe disease, mucopolysaccharidosis type II, fragile X syndrome, STXBP1 encephalopaihy, Krabbe disease, Huntington's disease, Alzheimer's disease, Battens disease, lysosomal storage disorders, glioblastoma multiforme, Rett syndrome, Leber's congenital amaurosis, Late infantile neuronal ceroid lipofuscinosis (LINCL), chronic pain, stroke, spinal cord injury, traumatic brain injury, lysosomal storage disorders, and/or cardiovascular disorders.

In some cases, the disease or condition is localized to a particular in vivo environment in the subject, e.g., the brain, spinal cord, and/or heart. The compositions of the present disclosure are particularly useful for the treatment of the diseases or conditions described herein because they specifically or more efficiently target the in vivo environment and deliver a therapeutic nucleic acid engineered to modulate the activity or the expression of a target gene expression product involved with the pathogenesis or pathology of the disease or condition.

Provided herein are methods of treating a disease or a condition, or a symptom of the disease or condition, in a subject, comprising: (a) diagnosing a subject with a disease or a condition affecting a target in vivo environment; and (b) treating the disease or the condition by administering to the subject a therapeutically effective amount of a composition disclosed herein (e.g., rAAV particle, AAV vector, pharmaceutical composition), wherein the composition is engineered with an increased expression.

Disclosed herein are methods of treating a disease or a condition, or a symptom of the disease or the condition, afflicting a target in a subject comprising: (a) administering to the subject a composition (e.g., rAAV particle, AAV vector, pharmaceutical composition); and (b) expressing the therapeutic nucleic acid into a target in vivo environment in the subject with an increased transduction enrichment.

In some embodiments, methods further comprise reducing or ablating delivery of the heterologous nucleic acid in an off-target in vivo environment, such as the liver. In some embodiments, delivery is characterized by an increase in enrichment of transduction (e.g., of the heterologous nucleic acid) in the brain, spinal cord, and/or heart.

In some embodiments, methods of treating a disease or condition affecting the brain, spinal cord, and/or heart comprise administering a rAAV particle to a brain, spinal cord, and/or heart in a subject, the rAAV particle comprising an rAAV capsid protein comprising an insertion of about, five, six, or seven amino acids of an amino acid sequence provided in Table 1, FIG. 1 and/or Formulas I-III, at an amino acid position 587-588 in a parental AAV2 capsid protein. In some embodiments, methods of treating a disease or condition affecting the brain, spinal cord, and/or heart comprise administering a rAAV particle to a brain, spinal cord, and/or heart in a subject, the rAAV particle comprising an rAAV capsid protein comprising an insertion of about, five, six, or seven amino acids of an amino acid sequence as well as one or more substitution at amino acid found at amino acid positions 586-589 such as provided in Table 1, FIG. 1 and/or Formulas I-III. In some embodiments, the parental AAV capsid protein is an AAV2 capsid protein (e.g., as provided in SEQ ID NO: 1).

Also provided are methods of modulating a target gene expression product, the methods comprising administering to a subject in need thereof a composition (e.g., rAAV particle, AAV vector, pharmaceutical composition) disclosed herein. For example, methods provided herein comprise administering to a subject a rAAV with a rAAV capsid protein encapsidating a viral vector comprising a heterologous nucleic acid that modulates the expression or the activity of the target gene expression product.

The term “normal individual” refers to an individual that is not afflicted with the disease or the condition characterized by the variation in expression or activity of the gene or gene expression product thereof.

In some embodiments, the disease or condition of the CNS, brain, and/or spinal cord is selected from Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavemous Malformation, Cerebral Gigantism, Cerebral Hypoxia, Cerebral Palsy, Cerebro-Oculo-Facio-Skeletal Syndrome (COFS), Charcot-Marie-Tooth Disease, Charcot-Marie-Tooth syndrome, classical rhizomelic chondrodysplasia punctata (RCDP), Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, Deafness, De Morsier's Syndrome, Dejerine-Klumpke Palsy, Dementia, Dementia—Multi-Infarct, Dementia-Semantic, Dementia-Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Duchenne muscular dystrophy, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X syndrome, Friedreich's Ataxia, Frontotemporal Dementia (FTD), Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, glioblastoma, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease (IRD), Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Maple syrup urine disease, Megalencephaly, Melkersson-Rosenthal Syndrome, Meningitis, Meningitis and Encephalitis, Menkes Disease, Menkes syndrome, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Mucopolysaccharidosis II, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Myotonic dystrophy, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry—Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phenylketonuria, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Prader-Willi syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar ataxia, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, STXBP1 encephalopathy, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tangier disease, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central Nervous Systems, Von Economo's Disease, Von Hippel-Lindau Disease (VHL), Von Hippel-Lindau syndrome, Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy and Zellweger syndrome.

In some embodiments, the pharmaceutical formulation comprises a therapeutic nucleic acid encoding a therapeutic gene expression product. In some instances, the therapeutic gene expression product is effective to modulate an activity or an expression of a target gene or gene expression product selected from ATP1A2, CACNAIA, SETD5, SHANK3, NF2, DNMT1, TCF4, RAI1, PEX1, ARSA, EIF2B5, EIF2B1, EIF2B2, NPC1, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMN), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SODI), iduronate 2 sulfatase (hIDS), Glucosylceramidase Beta (GBA), fragile X mental retardation 1 (FMR1), NPC Intracellular Cholesterol Transporter 1 (NPCl), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11, PEX12, PEX13, PEX14, PEX16, PEX19, and PEX26.

In some aspects, other examples of genes involved in CNS, brain, and/or spinal cord diseases or disorders include MAPT, IDUA, SNCA, ATXN2, Ube3a, GNS, HGSNAT, NAGLU, SGSH, CLN1, CLN3, CLN4, CLN5, CLN6, CLN7, CLN8, CTSD, ABCD1, HEXA, HEXB, ASM, ASPA, GLB1, AADC, MFN2, GNAO1, SYNGAP1, GRIN2A, GRIN2B, KCNQ2, EPM2A, NHLRC1, SLC6A1, SLC13A5, SURF1, GBE1, ATXN1, ATXN3, and ATXN7.

In some embodiments, the disease or condition of the heart includes any disease of the cardiopulmonary system, including, but not limited to heart failure, ischemia, arrhythmia, myocardial infarction, congestive heart failure, transplant rejection, abnormal heart contractility, non-ischaemic cardiomyopathy, mitral valve regurgitation, refractory myocardial ischaemia, non-ischaemic heart failure, aortic stenosis or regurgitation, abnormal Ca2+ metabolism, dysregulation of a heart-specific protein or a protein effective in modulating cardiac activity or physiology.

In some embodiments, genes involved in heart disease include those the express proteins involved in the regulation of calcium cycling in cardiomyocytes, such as a sarcoplasmic endoplasmic reticulum Ca2+ ATPase pump.

In some embodiments, cells of the heart or heart tissue comprise tissue or cells of any portion of the cardiopulmonary system. In certain aspects, cells of the heart or heart tissue comprises cardiac muscle cells/tissue, cells/tissue of the cardiac vasculature, and cells/tissue present in a cardiac valve. Cells of the heart may include cardiomyocytes, epithelial cells, endothelelial cells, fibroblasts, cells of the conducting tissue, cardiac pacemaking cells, and neurons.

In some instances, the therapeutic gene expression product comprises gene editing components. In some instances, the gene editing components are selected from an artificial site-specific RNA endonuclease (ASRE), a zinc finger endonuclease (ZFN), a transcription factor like effector nuclease (TALEN), a clustered regularly interspaced short palindromic repeats (CRISPR)/Cas enzyme, and a CRISPR)/Cas guide RNA.

In some instances, the expression of a gene or expression or activity of a gene expression product is inhibited by the administration of the composition to the subject. In some instances, the expression of a gene or the expression or the activity of a gene expression product is enhanced by the administration of the composition to the subject.

### Formulations, Dosages, and Routes of Administration

Disclosed herein are methods comprising delivering a rAAV particle encapsidating a heterologous nucleic acid to the brain, spinal cord, and/or heart in a subject, the rAAV particle comprising (i) an increased transduction of the heterologous nucleic acid in the brain, spinal cord, and/or heart, wherein the rAAV particle has an rAAV capsid protein comprising an insertion of five, six, or seven amino acids of an amino acid sequence provided in Table 1, FIG. 1 and/or Formulas I-III, at an amino acid position 587-588 in a parental AAV2 capsid protein or comprising an rAAV capsid protein comprising an insertion of about, five, six, or seven amino acids of an amino acid sequence as well as one or more substitution at amino acid found at amino acid positions 586-589, such as provided in Table 1, FIG. 1 and/or Formulas I-III.

In general, methods disclosed herein comprise administering a therapeutic rAAV composition by systemic administration. In some instances, methods comprise administering a therapeutic rAAV composition by intravenous (“i.v.”) administration. One may administer therapeutic rAAV compositions by additional routes, such as subcutaneous injection, intramuscular injection, intradermal injection, transdermal injection, percutaneous administration, intranasal administration, intralymphatic injection, rectal administration intragastric administration, intraocular administration, intracerebroventricular administration, intrathecally, intracisternal, or any other suitable parenteral administration. Routes, dosage, time points, and duration of administrating therapeutics may be adjusted. In some embodiments, administration of therapeutics is prior to, or after, onset of either, or both, acute and chronic symptoms of the disease or condition. Other routes of delivery include, but are not limited to intracranial administration, lateral cerebroventricular administration, and endovascular administration.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g., individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some cases, a dose of the pharmaceutical composition may comprise a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases, the concentration of infectious particles is 2×10⁷, 2×10⁸, 2×10⁹, 2×10¹⁰, 2×10¹¹, 2×10¹², 2×10³³, 2×10¹⁴, 2×10¹⁵, 2×10¹⁶, or 2×10¹⁷. In some cases, the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, or 3×10¹⁷. In some cases, the concentration of the infectious particles is 4×10⁷, 4×10, 4×10⁹, 4×10¹⁰, 4×10¹¹, 4×10¹², 4×10¹³, 4×10¹⁴, 4×10¹⁵, 4×10¹⁶, or 4×10¹⁷. In some cases, the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, or 5×10¹⁷. In some cases, the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, or 6×10¹⁷. In some cases, the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10¹⁶, or 7×10¹⁷. In some cases, the concentration of the infectious particles is 8×10⁷, 8×10¹, 8×10⁹, 8×10¹⁰, 8×10¹¹, 8×10¹², 8×10¹³, 8×10¹⁴, 8×10¹⁵, 8×10¹⁶, or 8×10¹⁷. In some cases, the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10^{1G}, or 9×10¹⁷.

Disclosed herein, in some embodiments are formulations of pharmaceutically-acceptable excipients and carrier solutions suitable for delivery of the rAAV compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, the pharmaceutical forms of the rAAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

In some cases, for administration of an injectable aqueous solution, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous or intrathecal administration.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of rAAV compositions and time of administration of such compositions will be within the purview of the skilled artisan having benefit of the present teachings. It is likely, however, that the administration of therapeutically-effective amounts of the disclosed compositions may be achieved by a single administration, example, a single injection of sufficient numbers of infectious particles to provide therapeutic benefit to the patient undergoing such treatment. This is made possible, at least in part, by the fact that certain target cells (e.g., cells of the brain, spinal cord, and/or heart) do not divide, obviating the need for multiple or chronic dosing.

In certain embodiments, the data obtained from cell culture assays and animal studies are used in formulating the therapeutically effective daily dosage range and/or the therapeutically effective unit dosage amount for use in mammals, including humans. In certain embodiments, the dosage range and/or the unit dosage amount varies within this range depending upon the dosage form employed and the route of administration utilized.

### Combination Therapies

A therapeutic rAAV may be used alone or in combination with an additional therapeutic agent (together, “therapeutic agents”). In some cases, a therapeutic rAAV as used herein is administered alone. The therapeutic agent may be administered together or sequentially in a combination therapy. The combination therapy may be administered within the same day, or may be administered one or more days, weeks, months, or years apart.

The additional therapeutic agent can comprise a small molecule. The additional therapeutic agent can comprise an antibody, or antigen-binding fragment. The additional therapeutic agent can include lipid nanoparticle-based therapies, anti-sense oligonucleotide therapies, as well as other viral therapies.

The additional therapeutic agent can comprise a cell-based therapy. Exemplary cell-based therapies include without limitation immune effector cell therapy, chimeric antigen receptor T-cell (CAR-T) therapy, natural killer cell therapy and chimeric antigen receptor natural killer (NK) cell therapy. Either NK cells, or CAR-NK cells, or a combination of both NK cells and CAR-NK cells can be used in combination with the methods disclosed herein. In some embodiments, the NK cells and CAR-NK cells are derived from human induced pluripotent stem cells (iPSC), umbilical cord blood, or a cell line. The NK cells and CAR-NK cells can comprise a cytokine receptor and a suicide gene. The cell-based therapy can comprise a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

### Kits

Disclosed herein are kits comprising compositions disclosed herein. Also disclosed herein are kits for the treatment or prevention of a disease or conditions of the brain, spinal cord, and/or heart. In some instances, the disease or condition is cancer, a pathogen infection, pulmonary disease or condition, neurological disease, muscular disease, or an immune disorder, such as those described herein.

In one embodiment, a kit can include a therapeutic or prophylactic composition containing an effective amount of a composition of a rAAV particle encapsidating a recombinant AAV vector encoding a therapeutic nucleic acid (e.g., therapeutic nucleic acid) and a recombinant AAV (rAAV) capsid protein of the present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some instances, the kit further comprises a cell. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the kit further comprises an AAV vector comprising a heterologous nucleic acid encoding a therapeutic gene expression product. In some instances, the AAV vector is an episome.

In some cases, rAAV are provided together with instructions for administering the rAAV to a subject having or at risk of developing the disease or condition (e.g., disease of the brain, spinal cord, and/or heart). Instructions can generally include information about the use of the composition for the treatment or prevention of the disease or condition.

In some cases, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some cases, instructions provide procedures for administering the rAAV to the subject alone. In some instances, the instructions provide that the rAAV is formulated for systemic delivery.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating skin disorders like acne, eczema, psoriasis, and rosacea.

The terms “homologous,” “homology,” or “percent homology” are used herein to generally mean an amino acid sequence or a nucleic acid sequence having the same, or similar sequence to a reference sequence. Percent homology of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “subject” is any organism. In some instances, the organism is a mammal. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In one embodiment, a “non-human primate” is a mammal, for example a monkey. In some instances, the subject is a patient, which as used herein, may refer to a subject diagnosed with a particular disease or disorder.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence.

The term “adeno-associated virus,” or “AAV” as used herein refers to the adeno-associated virus or derivatives thereof. Non-limited examples of AAV's include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), avian AAV, bovine AAV, canine AAV, equine AAV, primate AAV, non-primate AAV, and ovine AAV. In some instances, the AAV is described as a “Primate AAV,” which refers to AAV that infect primates. Likewise, an AAV may infect bovine animals (e.g., “bovine AAV”, and the like). In some instances, the AAV is wildtype, or naturally occurring. In some instances, the AAV is recombinant.

The term “AAV capsid” as used herein refers to a capsid protein or peptide of an adeno-associated virus. In some instances, the AAV capsid protein is configured to encapsidate genetic information (e.g., a transgene, therapeutic nucleic acid, viral genome). In some instances, the AAV capsid of the instant disclosure is a modified AAV capsid, relative to a corresponding parental AAV capsid protein.

The term “tropism” as used herein refers to a quality or characteristic of the AAV capsid that may include an increase or a decrease in enrichment of, expressing the encapsidated genetic information into an in vivo environment, relative to a second in vivo environment. An in vivo environment, in some instances, is a cell-type. An in vivo environment, in some instances, is an organ or organ system.

The term “AAV vector” as used herein refers to nucleic acid polymer encoding genetic information related to the virus. The AAV vector may be a recombinant AAV vector (rAAV), which refers to an AAV vector generated using recombinatorial genetics methods. In some instances, the rAAV vector comprises at least one heterologous polynucleotide (e.g., a polynucleotide other than a wild-type or naturally occurring AAV genome such as a transgene).

The term “AAV particle” as used herein refers to an AAV virus, virion, AAV capsid protein or component thereof. In some cases, the AAV particle is modified relative to a parental AAV particle.

The term “gene product” of “gene expression product” refers to an expression product of a polynucleotide sequence such as, for e.g., a polypeptide, peptide, protein or RNA, including interfering RNA (e.g., siRNA, miRNA, shRNA) and messenger RNA (mRNA).

The term “heterologous” as used herein refers to a genetic element (e.g., coding region) or gene expression product (e.g., RNA, protein) that is derived from a genotypically distinct entity from that of the rest of the entity to which it is being compared.

The term “endogenous” as used herein refers to a genetic element (e.g., coding region) or gene expression product (e.g., RNA, protein) that is naturally occurring in or associated with an organism or a particular cell within the organism.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, systemic administration.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. Alternatively, a sample can be obtained through primary patient derived cell lines, or archived patient samples in the form of preserved samples, or fresh frozen samples.

The term “in vivo” is used to describe an event that takes place in a subject's body.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “CNS” or “central nervous system” means a tissue selected from brain, thalamus, cortex, putamen, lateral ventricles, medulla, the pons, the amygdala, the motor cortex, caudate, hypothalamus, striatum, ventral midbrain, neocortex, basal ganglia, hippocampus, cerebrum, cerebellum, brain stem, and spinal cord. The brain includes a variety of cortical and subcortical areas, including the frontal, temporal, occipital and parietal lobes.

The term “systemic delivery” is defined as a route of administration of medication or other substance into a circulatory system so that the entire body is affected. Administration can take place via enteral administration (absorption of the drug through the gastrointestinal tract) or parenteral administration (generally injection, infusion, or implantation). “Circulatory system” includes both blood or cerebrospinal fluid circulatory systems. Examples of systemic administration for the CNS include intraarterial, intravenous or intrathecal injection. Other examples include administration to the cerebrospinal fluid at any location, in the spine (i.e., but not limited to lumbar) or brain (i.e., but not limited to cisterna magna). The terms “systemic administration” and “systemic delivery” are used interchangeably.

The section headings used herein are for organizational purposes only and are not to be construed as limiting the subject matter described.

### EXAMPLES

### Example 1

### Method of Identifying the Modified Capsid Proteins in Cynomolgus Macaques

Of primary concern for the therapeutic applicability of engineered adeno-associated viruses (AAVs) is how well their transduction profiles, including tissue-specific transduction profiles, translate to human application. While previous engineering efforts have focused on in vitro or in vivo rodent screening platforms due to the ease and flexibility of their use, screening efforts directly in non-human primates (NHPs) are much more likely to identify viruses that translate. The present Inventors chose Cynomolgus Macaques, an old-world NHP, for our engineering efforts. We focused our engineering efforts on a region of the AAV2 capsid surface located at amino acid position #587, one of the most exposed loops on the capsid surface that is a variable region between natural AAV serotypes and has a role in receptor binding. Insertion of peptides between positions 587 and 588 and between 588 and 589 has been studied in the past by us, and others, and has resulted in novel receptor binding and drastically altered capsid tropism. A library of viral capsids was created by performing a random 7 amino acid insertion at this site within AAV2 in hopes of achieving a novel tropism toward the NHP brain, heart, and/or spinal cord. This first library was introduced into the macaques, and the variants' expression levels were screened in different tissue types (i.e., the brain, spinal cord, heart, and liver). Of the billions of variants introduced in the first library, 39,000 variant sequences were identified for a second round of screening. As with the first round, the tissue-specific expression for these 39,000 were studied, and the best 975 were selected for inclusion in the variants described in FIG. 1 herein.

Plasmids. The first-round viral DNA library was generated by amplification of a section of the AAV2 capsid genome between amino acids 450-599 using NNK degenerate primers (Integrated DNA Technologies, Inc., IDT) to insert seven random amino acids between amino acids 587 and 588 with all possible variations. The resulting library inserts were then introduced into the rAAV-ΔCap-in-rev-RNA plasmid via Gibson assembly as previously described. The resulting capsid DNA library, rAAV-Cap-Cag-GFP 11, contained a diversity of billions of variants at the amino acid level. As explained, the second round viral DNA library comprised 39,000 variants at the amino acid level, with 3 barcoded replicates for each variant, which were selected from the variants of the round 1 screening.

The AAV2 REP-AAP-ACAP plasmid transfected into HEK293T cells to provide the Rep gene for library viral production prevents production of a wild-type AAV2 capsid during viral library production after a plausible recombination event between this plasmid co-transfected with the library plasmids at each stage containing the library inserts.

Viral production. Recombinant AAVs were generated according to established protocols. Briefly, immortalized HEK293T cells (ATCC) were quadruple transfected with four vectors using polyethylenimine (PEI). The first vector was the rAAV-Cap-in-cis-Lox library flanked by inverted terminal repeat (ITR) sequences from a parental AAV virus. The second vector was the AAV2/9 REP-AAP-ACAP plasmid. The third vector contains nucleic acids encoding helper virus proteins needed for viral assembly and packaging of the heterologous nucleic acid into the modified capsid structure. The fourth is a pUC-18 plasmid included to achieve the right PEI/DNA ratio for optimal transfection enrichment. Only 10 ng of rAAV-Cap-in-cis-Lox library DNA was transfected (per 150 mm plate) to decrease the likelihood of multiple library DNAs entering the same cell. Viral particles are harvested from the cells and media after 60 h post transfection. Virus present in the media is concentrated by precipitation with 8% polyethylene glycol and 500 mM sodium chloride and the precipitated virus is added to the lysates prepared from the collected cells. The viruses are purified over iodixanol (Optiprep, Sigma) step gradients (15%, 25%, 40%, and 60%). Viruses are concentrated and formulated in PBS. Virus titers are determined by measuring the number of DNaseI-resistant vector genome copies (VGs) using qPCR and the linearized genome plasmid as a control.

Animals. Cynomolgus macaque procedures were approved by the IACUC committee of Envol Biomedical. Cynomolgus macaques were born and raised in Envol Biomedical colonies and housed in family groups under standard conditions. They were fed ad libitum and received enrichment as part of the primate enrichment program for NHPs at Envol Biomedical. For AAV infusions, animals were screened for endogenous neutralizing antibodies (Nab). None of the animals that were screened showed any detectible blocking reaction at 1:10 dilution of serum. They were then group housed for several days and acclimated to a new room before injections. Animals were restrained and the test article was administered via intravenous infusion for 10 minutes. Two juvenile monkeys were used in each of the round 1 and round 2 screenings. Activity and behavior were monitored daily over the course of in-life.

DNA/RNA recovery and sequencing. Round 1 and round 2 viral libraries were injected into cynomolgus macaques at a dose of 1-3×10¹³ vg/kg animal and rAAV genomes were recovered two weeks post injection. Animals were euthanized and brain, spinal cord, heart, and liver were recovered, snap frozen, and placed into long-term storage at −80° C. as well as other peripheral tissues. For round 1, the brain was separated into seven brain regions, 100 mg each, and for round 2, thirteen brain regions. 20-100 mg of each brain section, spinal cord, and liver was homogenized in buffer using the MagMAX DNA ULTRA (A25597) and a Bead Ruptor 96 (OMNI, INC) and viral DNA was isolated according to the manufacturers recommended protocol. Recovered viral DNA was treated with RNase, and purified with a Zymo DNA Clean and Concentrator kit (D4033). Viral genomes were enriched by 25 cycles of PCR amplification with primers flanking the 587-588 insertion site in the capsid genome using 50% of the total extracted viral DNA as a template. After Zymo DNA purification, samples were diluted 1:10 to 1:1000 depending on tissue type and each dilution further amplified around the library variable region with 10 cycles of PCR. Subsequently, samples were further amplified using custom primers with Illumina Indices for 10 more cycles. The amplification products were run on a 2% low-melting point agarose gel (ThermoFisher Scientific, 16520050) for better separation and recovery of the 210 bp band.

For the second round library only, packaged viral library DNA was isolated from the injected viral library by digestion of the viral capsid and purification of the contained ssDNA. These viral genomes were amplified by two PCR amplification steps, like the viral DNA extracted from tissue, to add adapters and indices for Illumina next-generation sequencing, and purified after gel electrophoresis. This viral library DNA, along with the viral DNA extracted from tissue, was sent for deep sequencing using an Illumina NextSeq 2000 system.

NGS data alignment and processing. Raw fastq files from NGS runs were processed with custom-built scripts (Capsida CapSeq Tools). For the first round library, the pipeline to process these datasets involved filtering to remove low-quality reads, utilizing a quality score for each sequence, and eliminating bias from PCR-induced mutations or high GC-content. The filtered dataset was then aligned by a perfect string match algorithm and trimmed to improve the alignment quality. Read counts for each sequence were pulled out and displayed by tissue, at which point all sequences found in the brain, spinal cord, and/or heart were compiled for formation of the second round library.

For the second round, library read counts by tissue were similarly tabulated. Then, a read count of 1 was added to each sequence to remove 0 values, all brain regions for each sequence were summed together, and the read sequences for each codon replicate of a given 7-mer amino acid sequence were summed together to give a single value for each peptide insertion. Finally, the read count data was normalized to counts per million (Cpm). Enrichment values for each capsid variant were calculated via normalized cpm from [tissue of interest]/cpm from viral library.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

1. An AAV capsid protein comprising a sequence having at least 70% identity with a sequence selected from SEQ ID NOS: 976-1949.

2. The AAV capsid protein of claim 1, comprising a sequence having at least 80% identity with a sequence selected from SEQ ID NOS: 2-975.

3. The AAV capsid protein of claim 2, comprising a sequence having at least 90% identity with a sequence selected from SEQ ID NOS: 2-975.

4. The AAV capsid protein of claim 3, comprising a sequence selected from SEQ ID NOS: 2-975.

5. The AAV capsid protein of claim 1, wherein the capsid protein comprises a seven-amino-acid insertion having a sequence having at least 70% identity with a sequence selected from one of SEQ ID NOS: 976-1949 in an amino acid sequence having a sequence at least 98% identical to amino acid 217 to amino acid 736 of the AAV capsid protein provided in SEQ ID NO: 1.

6. The AAV capsid protein of claim 5, wherein the seven-amino-acid insertion is inserted between amino acid position 587 and 588 of SEQ ID NO: 1.

7. The AAV capsid protein of claim 6, wherein the AAV capsid protein is characterized by at least one of an increased specificity and/or an increased transduction efficiency when measured in the brain, heart, or spinal cord in a subject when delivered to the subject systemically, relative to a native AAV capsid protein of SEQ ID NO: 1

8. The AAV capsid protein of claim 7, wherein the AAV capsid protein is characterized by an increased specificity and/or increased transduction efficiency when measured in the brain relative to the capsid protein of SEQ ID NO: 1.

9. The AAV capsid protein of claim 8, wherein the seven-amino-acid insertion comprises a sequence having at least 70% identity with a sequence selected from one of SEQ ID NOS: 976-1411.

10. The AAV capsid protein of claim 9, wherein the seven-amino-acid insertion comprises a sequence of Formula [I]:
(SEQ ID NO. 1950)
X¹-X²-X³-X⁴-X⁵-X⁶-X⁷ (Formula I),
X¹ is an amino acid selected from L, I, and V;
X² is an amino acid selected from L, V, and M;
X³ is an amino acid selected from G, S, and P;
X⁴ is an amino acid selected from P, G, S, T, and H;
X⁵ is an amino acid selected from S, L, T, A, H, and Q;
X⁶ is an amino acid selected from L, M, P, S, T, and G; and/or
X⁷ is an amino acid selected from S, T, A, L, Q, P, and V.

11. The AAV capsid protein of claim 10, wherein X² is L.

12. The AAV capsid protein of claim 10, wherein X³ is G.

13. The AAV capsid protein of claim 10, wherein X⁶ is L.

14. The AAV capsid protein of claim 9, wherein the seven-amino-acid insertion is one of SEQ ID NOS: 976-1411.

15. The AAV capsid protein of claim 14, wherein the capsid protein comprises a sequence of one of SEQ ID NOS: 2-437.

16. The AAV capsid protein of claim 7, wherein the AAV capsid protein is characterized by an increased specificity and/or increased transduction efficiency when measured in the heart relative to the capsid protein of SEQ ID NO: 1.

17. The AAV capsid protein of claim 16, wherein the seven-amino-acid insertion comprises a sequence having at least 70% identity with a sequence selected from one of SEQ ID NOS: 1412-1612.

18. The AAV capsid protein of claim 17, wherein the seven-amino-acid insertion comprises a sequence of Formula [I]:
(SEQ ID NO. 1951)
X¹-X²-X³-X⁴-X⁵-X⁶-X⁷ (Formula [I]),
wherein:
X¹ is an amino acid selected from S, N, T, and L;
X² is an amino acid selected from T, N, V, and S;
X³ is an amino acid selected from R, K, T, and S;
X⁴ is an amino acid selected from K, S, T, A, and R;
X⁵ is an amino acid selected from L, I, S, R, and G;
X⁶ is an amino acid selected from P, S, Q, and L; and/or
X⁷ is an amino acid selected from P, A, and S.

19. The AAV capsid protein of claim 17, wherein the seven-amino-acid insertion is one of SEQ ID NOS: 1412-1612.

20. The AAV capsid protein of claim 19, wherein the capsid protein comprises a sequence of one of SEQ ID NOS: 438-638.

21.-44. (canceled)
